(19) United States
(12) Patent Application Publication
(10) Pub. No.: US 2023/0107291 A1
(43) Pub. Date: Apr. 6, 2023

(54) Method for Identifying Functional Disease-Specific Regulatory T Cells

(71) Applicants: Institut Curie, Paris (FR); Institut National de la Santé et de la Recherche Médicale, Paris (FR); Institut Mutualiste Montsouris, Paris (FR)

(72) Inventors: Eliane Piaggio, Paris (FR); Wilfrid Richer, Rueil Malmaison (FR); Christine Sedlik, Le Plessis Bouchard (FR); Jimena Tosello, Paris (FR); Joshua Waterfall, Paris (FR); Elisa Bonnin, Paris (FR)

(73) Assignees: Institut Curie, Paris (FR); Institut National de la Santé et de la Recherche Médicale, Paris (FR); Institut Mutualiste Montsouris, Paris (FR)

(21) Appl. No.: 17/801,014

(22) PCT Filed: Feb. 22, 2021
(86) PCT No.: PCT/EP2021/054355

§ 371 Date: Aug. 19, 2022

(30) Foreign Application Priority Data
Feb. 20, 2020 (EP) 20305167.7

(51) Int. Cl. C12Q 1/6809; C12N 5/0783; C12Q 1/6883; G01N 33/53

(52) U.S. Cl. CPC C12Q 1/6809; C12N 5/0637; C12Q 1/6883; G01N 33/53

(57) ABSTRACT

The invention relates to a method of identification of functional disease-specific, in particular tumor-specific, regulatory T cells and markers thereof. The invention also relates to the derived functional tumor-specific regulatory T cells, markers and engineered regulatory T cells and to their use for the diagnosis, prognosis, monitoring and treatment of cancer.

### FIELD OF THE INVENTION

The invention pertains to the field of immunotherapy, in particular of cancer. The invention relates to a method of identification of functional disease-specific, in particular tumor-specific, regulatory T cells and markers thereof. The invention also relates to the derived functional tumor-specific regulatory T cells, markers and engineered regulatory T cells and to their use for the diagnosis, prognosis, monitoring and treatment of cancer.

### BACKGROUND OF THE INVENTION

CD4+ Foxp3+ regulatory T cells (Tregs) play a critical role in the maintenance of immune homeostasis and actively suppress immune responses to self, tumors, microbes and grafts (Sakaguchi et al., Int. Immunol., 2009, 21, 1105-1111). So, understanding the biology and function of Tregs is a key challenge for immunologists and a prerequisite for improving current approaches for the diagnosis, prognosis, monitoring and treatment of diseases, in particular cancer.

Elevated frequencies of Tregs are found in many human cancers and are associated with poor clinical outcomes. In mouse models, manipulation of Tregs has given impressive results. On one side, adding therapeutic Tregs or boosting endogenous Tregs was shown to dampen autoimmunity (Churlaud et al., Clin. Immunol. Orlando Fla., 2014, 151, 114-126; Gringer-Bleyer et al., J. Clin. Invest., 2010, 120, 4558-4568) or inflammation (Gaidot et al., Blood, 2011, 117, 2975-2983; Perol et al., Immunol. Lett., Dutch Society for Immunology, 2014, 162, 173-184). On the other side, depleting/inactivating Tregs has proven very valuable to increase anti-tumor (Alonso et al., Nat. Commun., 2018, 9, 2113; Caudana et al., Cancer Immunol. Res., 2019, 7, 443-457; Fontenot et al., Nat. Immunol., 2003, 4, 330-336) or anti-vaccine responses. Therefore, therapeutic strategies targeting Tregs have been proposed for cancer treatment including non-exhaustively: (i) Treg cell-based approaches comprising injection of Treg-depleted donor lymphocyte after hematopoietic stem cell transplantation for the treatment of hematological malignancies (Maury et al., Sci. Transl. Med., 2010, 2, 41ra52-41ra52) and (ii) approaches inducing selective depletion or functional alteration of Treg cells, including; chemical drugs modulating Treg-associated pathways, like cyclophosphamide (Lutsiak et al., Blood, 2005, 105, 2862-2868), fludarabine, gemcitabine, and mitoxantrone (Dwarakanath et al., Cancer Rep., 2018, 1, e21105; Wang et al., Cell Rep., 2018, 23, 3262-3274); Treg-depleting antibodies (like anti-CTLA-4, anti-CD25, anti-CCR5, anti-CCR4; Dwarakanath et al., Cancer Rep., 2018, 1, e21105); Cytokines and modified cytokines including for example high dose IL-2 (to stimulate effector cells in cancer), and IL-2-derivatives with specific selectivity to Tregs or effector cells (IL-2/anti-IL-2 complexes, pegylated IL-2; resurfaced IL-2 variants (Pero′, L., Piaggio, E., 2016. New Molecular and Cellular Mechanisms of Tolerance: Tolerogenic Actions of IL-2, in: Cuturi, M. C., Anegon, I. (Eds.), Suppression and Regulation of Immune Responses. Springer New York, N.Y., N.Y., pp. 11-28).

Nevertheless, cell-based therapies are very expensive and cumbersome; and although pre-clinical data have given a solid rational to use the above-mentioned approaches, and some are under clinical evaluation, there still remains a medical need to discover effective and selective Treg-targeted immunotherapies for the treatment of autoimmune/inflammatory diseases, as well as cancer.

One of the main hurdles that have precluded translation into the clinic is the difficulty in identifying unique Treg markers. Indeed, Tregs express high levels of CD25 and Foxp3 (Hori et al., Science, 2003, 299, 1057-1061; Tran et al., Blood, 2007, 110, 2983-2990), but conventional human CD4+ T cells (Tconvs) can also acquire CD25 and Foxp3 upon activation, so there is a big overlap in the phenotype of Tregs and activated Tconvs (Tran et al., Blood, 2007, 110, 2983-2990).

In addition, Tregs constitute a heterogeneous population shaped by microenvironmental cues (Campbell and Koch, Nat. Rev. Immunol., 2011, 11, 119-130; Feuerer et al., Nat. Immunol., 2003, 4, 330-336). Indeed, as studies of Treg transcriptomic signatures emerged, it became apparent that Tregs do not possess a unique molecular signature. Indeed, at the steady state, the unique molecular patterns of Tregs obtained from different tissues (blood, lymphoid tissues, non-lymphoid tissues) suggest that Tregs can readily respond to the surrounding microenvironment, acquiring different migration capacities, activating different functional and metabolic pathways, and displaying diverse functions; defining distinct Treg subpopulations.

Furthermore, the inflammatory milieu associated to different pathologies can distinctly affect the Treg molecular profile and associated functions (Burzyn et al., Nat. Immunol., 2013, 14, 1007-1013; Chaudhry et al., Science, 2009, 326, 986-991; Zhou et al., Nat. Immunol., 2009, 10, 1000-10074). Consequently, to efficiently manipulate Tregs for therapeutic aims, it is mandatory to understand the unique Treg traits associated to each pathology.

Tregs and human cancer is indeed a big conundrum to solve. Tregs present in the tumor can be of different origins and suppress by multiple mechanisms. Growing data in the literature suggest that tumor-Tregs can boost cancer progression by diverse mechanisms, ranging from direct inhibition of effector T and NK cells and re-programming of myeloid cell into tolerogenic cells, to the induction of the production of inhibitory molecules (e.g. VEGF, IDO, prostaglandins) by different stromal cells, overall imprinting a suppressive tumor-microenvironment. Furthermore, tumor-specific Tregs can originate in the thymus (tTregs) or they can arise from conversion of naïve T cells into “peripheral-induced” Tregs (pTregs) (Lee, H.-M., Bautista, J. L., Hsieh, C.-S., 2011. Chapter 2—Thymic and Peripheral Differentiation of Regulatory T Cells, in: Alexander, R., Shimon, S. (Eds.), Advances in Immunology, Regulatory T-Cells. Academic Press, pp. 25-71; Lee et al., Exp. Mol. Med., 2018, 50, e456). Today, the distinction of tTregs from pTregs is limited to the use of only few markers with limited specificity (Helios, Nrp-1, CD31, Fopx3 promoter methylation) (Lin et al., J. Clin. Exp. Pathol., 2013, 6, 116-123). Whether tumor-specific Tregs are tTreg or pTregs remains unknown. Understanding the unique characteristics of tTregs and pTregs should give new possibilities to finely manipulate tumor-Tregs for therapeutic purposes.

Information on cancer-associated Treg biology in humans is limited. Studies on Treg cells in different cancer types indicate that: i) the proportion of FOXP3+CD4+ Tregs in the blood of cancer patients is increased compared to healthy donors (Liyanage et al., J. Immunol., 2002, 169, 2756-2761; Wolf et al., Clin. Cancer Res., 2003, 9, 606-612), and ii) high proportions of FOXP3+CD4+ Tregs in the tumor are associated with a bad prognosis (Bates et al., J. Clin. Oncol., 2006, 24, 5373-5380; Mahmoud et al., J. Clin. Oncol., 2011, 29, 1949-1955; Merlo et al., J. Clin. Oncol., 2009, 27, 1746-1752; Mouawad et al., J. Clin. Oncol., 2011, 29, 1935-1936; Ohara et al, Cancer Immunol. Immunother., 2009, 58, 441-447; Sun et al., Cancer Immunol. Immunother., 2014, 63, 395-406).

Only recently the first bulk RNAseq analysis of Tregs purified from human tumors have been performed (Plitas et al., Immunity, 2016, 45, 1122-1134), and very recently, single-cell (sc) analysis of Tregs purified from human tumors was performed (De Simone et al., Immunity, 2016, 45, 1135-1147). Even more recently, sc data on the association of the T-cell transcriptome and TCR were first reported for liver, breast, colorectal and non-small-cell lung cancer (Azizi et al., Cell, 2018, 174, 1293-1308; Guo et al., Nat. Med., 2018, 24, 978; Zemmour et al., Nat. Immunol. 19, 2018, 291-301; Zhang et al., Nature, 2018, 564, 268). These types of studies have revealed unprecedented heterogeneity among Treg cells both in normal and pathologic conditions making tumor-specific Treg analysis a technically difficult task for scientists. Furthermore, in these studies relatively low-numbers of Tregs were analyzed giving a low power to detect or define tumor-specific Treg cells and a low level of resolution in the definition of tumor-specific Treg cells.

Notwithstanding, immune modulation of the immune response to tumors occurs not only during the effector T cell phase in the tumor bed, but also, at the level of T-cell priming in the tumor-draining lymph nodes (TDLNs) (Chen and Mellman, Immunity, 2013, 39, 1-10). Of importance, although Tregs present in TDLNs will largely shape the anti-tumoral T-cell response, data on the phenotype and function of the Treg cells present in the TDLN of cancer patients is very limited (Faghig et al., Immunol. Lett., 2014, 158, 57-65; Gupta et al., Cancer Invest., 2011, 29, 419-425; Kohrt et al., PLOS Med., 2005, 2, e284; Nakamura et al., Eur. J. Cancer, 2009, 45, 2123-2131; Zuckerman et al., Int. J. Cancer, 2013. 132, 2537-2547).

Therefore, reliable methods for identifying tumor-specific Tregs and reliable tumor-specific Treg markers are missing for the treatment of cancer and other diseases.

The invention solves this problem by providing a method of identification of functional disease-specific regulatory T cells, in particular functional tumor-specific regulatory T cells, and markers thereof. The invention also provides functional tumor-specific regulatory T cells and Treg markers identified by the method including biomarkers and candidate therapeutic targets which are useful for the diagnosis, prognosis, monitoring and treatment of cancer. The invention further provides engineered Treg cells derived from said functional tumor-specific regulatory T cells and Treg markers.

### SUMMARY OF THE INVENTION

The inventors have used single-cell RNA sequencing of the transcriptome coupled to the TCR of Tregs and Tconvs from blood, tumor-draining lymph nodes (TDLNs) and tumors of cancer patients to classify Tregs in functional subsets and distinguish functional tumor-Treg clusters (FT-Tregs) out of the heterogeneous pool of Tregs. The FT-Treg clusters are identified as the clusters of Treg cells that accumulated in the tumor or tumor-draining lymph nodes (compared to blood), that are enriched in clonally expanded cells, and that are enriched in cells with transcriptomic features of TCR-mediated activation. TCRs are used as “molecular tags” to study FT-Treg clonal dynamic among the three tissues and complete the understanding of the tissue-adaptation of different Treg subpopulations, for the design of effective and selective approaches to manipulate FT-Tregs. Novel therapeutic targets (molecules or pathways) to specifically disable FT-Tregs and not all Tregs were identified by differential gene expression analysis, and targets were validated using Tregs knock-out for the candidate molecules and functional in vitro and/or in vivo tests to understand their role in Treg biology. The generated FT-Treg molecular targets can be used to guide the selection of candidate therapeutic strategies, including approaches based on cell-therapy, on antibodies, cytokines or chemical drugs that induce selective depletion or functional alteration of Treg cells. Selective inhibition of tumor-specific Tregs, while preserving effector T cells and Tregs from healthy tissues (that maintain immune homeostasis and control autoimmunity), represents a more effective and safer strategy that should lead to the enhancement of effective anti-tumor immunity, without eliciting generalized autoimmunity.

Also, the method could be applied as a research tool to characterize Tregs associated to any defined human pathology. This method could lead to the identification of Treg-associated molecules with potential value as biomarker of diagnosis, prognosis or toxicity. The understanding of the biological role of novel Treg-associated molecules that could be gained with this method could be used to design novel therapeutic strategies to improve vaccination approaches and to treat a broad range of immune-mediated pathologies, including autoimmune, inflammatory and immune-metabolic diseases, allergy, infectious diseases, GVHD, transplantation, foetus rejection and cancer.

Therefore, the invention relates to a method of identification of functional disease-specific regulatory T cell markers, comprising the steps of:
(a) Preparing a mixture of isolated regulatory T (Treg) cells and conventional T (Tconv) cells in similar proportions from at least a patient diseased-tissue sample and a patient peripheral blood sample;
(b) Performing single-cell gene expression profiling combined with T cell receptor (TCR) profiling on each mixture of isolated Treg and Tconv cells from at least diseased-tissue and peripheral blood;
(c) Identifying clusters of Treg cells and Tconv cells, wherein the clusters comprise differentially expressed genes or gene signatures between each other;
(d) Determining at least one cluster of functional disease-specific Treg cells among the identified clusters of Treg cells, wherein the at least one cluster comprises:
   (i) a higher proportion of Treg cells in the diseased-tissue than in the peripheral blood;
   (ii) a higher proportion of Treg cells with clonally expanded TCR specificities in the diseased-tissue; and
   (iii) a higher proportion of Treg cells with a transcriptomic signature of TCR triggering, cell activation and expansion in the diseased-tissue; and
(e) Identifying genes that are differentially expressed in the cluster of functional disease-specific Treg cells in comparison with all the other identified clusters of Treg and Tconv cells.

In some embodiments of the method of the invention, the patient diseased-tissue sample is patient tumor sample and/or the patient samples comprise a patient diseased-tissue sample, a patient tissue draining lymph node sample and a patient peripheral blood sample, in particular a patient tumor sample, a patient tumor draining lymph node sample and a patient peripheral blood sample.

In some preferred embodiments of the method of the invention, the mixture is composed of about 50% of Tconv cells and about 50% of Treg cells.

In some embodiments of the method of the invention, the combined single-cell gene expression profiling and T cell receptor (TCR) profiling in step (b) is performed by single-cell RNA sequencing method.

In some embodiments of the method of the invention, the at least one cluster of functional disease-specific Treg cells comprises a higher proportion of Treg cells overexpressing of one or more of: REL, NKKB2, NR4A1, OX-40, 4-1BB, MHC class II molecules, in particular HLA-DR; CD39, CD137 and GITR.

In some preferred embodiments of the method of the invention, said disease is cancer. Preferably, a cancer selected from the group comprising: non-small cell lung cancer (NSCLC); breast, skin, ovarian, kidney and head and neck cancers; and rhabdoid tumors; more preferably non-small cell lung cancer (NSCLC).

In some embodiments of the method of the invention, said disease is chosen from acute or chronic inflammatory, allergic, autoimmune or infectious diseases, graft-versus-host disease, and graft-rejection.

In some embodiments, the method of the invention, further comprises the identification and ranking of tumor-specific Treg markers for therapeutic purpose, according to the following steps:
Step 1: Identifying and selecting a fraction of n differentially expressed genes which code for a cell membrane protein; preferably a transmembrane or GPI-anchored protein with an extracellular domain;
Step 2: Determining the average expression level of the n selected genes in normal tissue and assigning at least one score A to each gene from −1 for the gene having the lowest expression level to −n for the gene having the highest expression level in normal tissue;
Step 3: Determining the average expression level of the n selected genes in tumoral tissue and assigning at least one score B to each gene from +n for the gene having the highest expression level to +1 for the gene having the lowest expression level in tumoral tissue;
Step 4: Determining the average expression level of the n selected genes in normal PBMCs except Tregs and assigning at least one score C to each gene from +n for the gene having the lowest expression level to +1 for the gene having the highest expression level in normal PBMCs except Tregs;
Step 5: Determining the average expression level of the n selected genes in the tumor environment except Tregs and assigning at least one score D to each gene from +n for the gene having the lowest expression level to +1 for the gene having the highest expression level in tumor environment except Tregs;
Step 6: Determining the relative expression level of the n selected genes in i) Tumor-Tregs compared to Normal tissue-Tregs, and ii) Tregs compared to Tconvs and assigning two scores E and F to each gene from +n for the gene having the highest fold change expression level to +1 for the gene having the lowest fold change in i) (score E) Tumor Treg compared to normal adjacent tissue Treg, and ii) (score F) Tregs compared to Tconvs;
Step 7: Summating the assigned scores to obtain a cumulative assessment value (SUM SCORE) for each gene; and
Step 8: Determining the candidate therapeutic targets based on the cumulative assessment value.

Another object of the invention is a molecular marker for the detection, inactivation or depletion of tumor-specific Treg cells identified by the method according to the present disclosure, which is selected from the genes of Table 1, and their RNA or protein products. In some particular embodiments, the molecular marker is a cell-surface marker selected from the group consisting of: ADORA2A, CALR, CCR8, CD4, CD7, CD74, CD80, CD82, CD83, CSF1, CTLA4, CXCR3, HLA-B, HLA-DQA1, HLA-DR, in particular HLA-DRB5, ICAM1, ICOS, IGFLR1, IL12RB2, IL1R2, IL21R, IL2RA, IL2RB, IL2RG, LRRC32, NDFIP2, NINJ1, NTRK1, SDC4, SLC1A5, SLC3A2, SLC7A5, SLCO4A1, TMPRSS6, TNFRSF18, TNFRSF1B, TNFRSF4, TNFRSF8, TNFRSF9, TSPAN13 and TSPAN17; or the molecular marker is VDR; preferably selected from the group consisting of: CCR8, CD80, ICOS, IL12RB2, CTLA-4, 4-1BB (TNFRS9), TNFRSF18 (GITR), HLA-DR, in particular HLA-DRB5, ICAM1, CSF1, CD74, OX-40 (TNFRSF4), CXCR-3, VDR and TNFR2 (TNFRSF1B); more preferably selected from the group consisting of: CD74, VDR, IL12RB2, HLA-DR, in particular HLA-DRB5, ICAM1 and CSF1.

In some embodiments, the molecular marker according to the present disclosure is a therapeutic target; preferably which modulate(s) the viability, proliferation, stability or suppressive function of functional tumor-specific Treg cells.

Another object of the invention is an agent for use as a Treg-inactivating or Treg-depleting agent in a method of treating cancer, wherein said agent is a modulator of the therapeutic target according to the present disclosure; preferably selected from the group comprising: small organic molecules, aptamers, antibodies, anti-sense oligonucleotides, interfering RNAs, ribozymes, and other agonists or antagonists such as for example dominant negative mutants or functional fragments of the therapeutic target protein.

In some embodiments, the agent is a cytotoxic agent comprising a molecule which binds to a tumor-specific Treg cell surface marker from Table 1, coupled to a cytotoxic compound. The molecule which binds to said tumor-specific Treg cell surface marker is preferably an antibody or a functional fragment thereof comprising the antigen binding site. The tumor-specific Treg cell surface marker from Table 1 is preferably selected from the above-listed tumor-specific Treg cell surface markers according to the present disclosure.

In some embodiments, the agent is for use to inactivate or deplete tumor-specific Treg cells in vivo or ex vivo.

Another object of the invention is an in vitro method of diagnosis, prognosis or monitoring of cancer, comprising the step of detecting the presence or level of expression of at least one molecular marker according to the present disclosure in a tumor sample from a subject and eventually also in a tumor draining lymph node sample from the subject; preferably wherein the method further comprises the step of classifying the subject into favorable or unfavorable outcome category based on the presence, absence or level of expression of said marker.

Another object of the invention is an engineered Treg cell defective for at least one of the up-regulated genes of Table 1 or which over-expresses at least one of the down-regulated genes of Table 1. In particular embodiments, the engineered Treg cell is defective for at least one the above-listed tumor-specific Treg cell surface markers according to the present disclosure.

In some embodiments, the engineered Treg cell further comprises at least one genetically engineered antigen receptor that specifically binds a target antigen.

### DETAILED DESCRIPTION OF THE INVENTION

### Definitions

As used herein, “regulatory T cells” or “Tregs” refer to CD4+ Foxp3+ cells.

As used herein, “functional disease-specific regulatory T cells” or “FD-Tregs” refer to a distinct population (or group, subset or cluster) of CD4+ Foxp3+ cells that distinguishes from the heterogeneous pool of Tregs in that: (i) it is increased in the diseased-tissue compared to the peripheral blood; (ii) it is enriched with clonally expanded TCR specificities in the diseased-tissue; and (iii) it is enriched with a transcriptomic signature of T cell Receptor (TCR) triggering, cell activation and expansion.

As used herein, “functional tumor-specific regulatory T cells” or “FT-Tregs” refer to a distinct and isolated population (or group, subset or cluster) of CD4+ Foxp3+ cells that distinguishes from the heterogeneous pool of Tregs in that: (i) it is increased in the tumor, and eventually also in the tumor draining-lymph node(s); (ii) it is enriched with clonally expanded TCR specificities in the diseased-tissue; and (iii) it is enriched with a transcriptomic signature of T cell Receptor (TCR) triggering, cell activation and expansion.

As used herein, «gene signature» or «gene expression signature» refers to a single or combined group of genes in a cell with a uniquely characteristic pattern of gene expression that occurs as a result of an altered or unaltered biological process or pathogenic medical condition.

The term “marker” as used herein means “molecular marker” or “molecular signature” and refers to a specific gene or gene product (RNA or protein). The term “marker” includes a biomarker and/or a therapeutic target.

As used herein, “biomarker” refers to a distinctive biological or biologically derived indicator of a process, event or condition.

As used herein, the term “disease” refers to any immune disorder such as with no limitations: acute or chronic inflammatory, allergic, autoimmune or infectious diseases, graft-versus-host disease, graft-rejection, and cancer.

As used herein, the term “cancer” refers to any member of a class of diseases or disorders characterized by uncontrolled division of cells and the ability of these cells to invade other tissues, either by direct growth into adjacent tissue through invasion or by implantation into distant sites by metastasis. Metastasis is defined as the stage in which cancer cells are transported through the bloodstream or lymphatic system. The term cancer according to the present invention also comprises cancer metastases and relapse of cancer. Cancers are classified by the type of cell that the tumor resembles and, therefore, the tissue presumed to be the origin of the tumor. For example, carcinomas are malignant tumors derived from epithelial cells. This group represents the most common cancers, including the common forms of breast, prostate, lung, and colon cancer. Lymphomas and leukemias include malignant tumors derived from blood and bone marrow cells. Sarcomas are malignant tumors derived from connective tissue or mesenchymal cells. Mesotheliomas are tumors derived from the mesothelial cells lining the peritoneum and the pleura. Gliomas are tumors derived from glia, the most common type of brain cell. Germinomas are tumors derived from germ cells, normally found in the testicle and ovary. Choriocarcinomas are malignant tumors derived from the placenta. As used herein, “cancer” refers to any cancer type including solid and liquid tumors.

The terms “subject” and “patient” are used interchangeably herein and refer to both human and non-human animals. As used herein, the term “patient” denotes a mammal, such as with no limitations a rodent, a feline, a canine, a bovine, an ovine, an equine and a primate. Preferably, a patient according to the invention is a human.

The term “patient sample” means any biological sample derived from a patient. Examples of such samples include fluids, tissues, cell samples, organs, biopsies. Preferred biological samples are tumor sample.

The term “treating” or “treatment”, as used herein, means reversing, alleviating, inhibiting the progress of, or preventing the disorder or condition to which such term applies, or reversing, alleviating, inhibiting the progress of, or preventing one or more symptoms of the disorder or condition to which such term applies. As used herein, the terms “treatment” or “treat” refer to both prophylactic or preventive treatment as well as curative or disease modifying treatment, including treatment of patients at risk of contracting the disease or suspected to have contracted the disease as well as patients who are ill or have been diagnosed as suffering from a disease or medical condition, and include suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment.

“Treating cancer” includes, without limitation, reducing the number of cancer cells or the size of a tumor in the patient, reducing progression of a cancer to a more aggressive form (i.e. maintaining the cancer in a form that is susceptible to a therapeutic agent), reducing proliferation of cancer cells or reducing the speed of tumor growth, killing of cancer cells, reducing metastasis of cancer cells or reducing the likelihood of recurrence of a cancer in a subject. Treating a subject as used herein refers to any type of treatment that imparts a benefit to a subject afflicted with cancer or at risk of developing cancer or facing a cancer recurrence. Treatment includes improvement in the condition of the subject (e.g., in one or more symptoms), delay in the progression of the disease, delay in the onset of symptoms, slowing the progression of symptoms and others.

As used herein, “drug” or “therapeutic agent” refers to a compound or agent that provides a desired biological or pharmacological effect when administered to a human or animal, particularly results in an intended therapeutic effect or response on the body to treat or prevent conditions or diseases. Therapeutic agents include any suitable biologically-active chemical compound or biologically derived component.

As used herein, a “therapeutic response” or “response to treatment with a drug” refers to a positive medical response characterized by objective parameters or criteria such as objective clinical signs of the disease, patient self-reported parameters and/or the increase of survival. The objective criteria for evaluating the response to drug-treatment will vary from one disease to another and can be determined easily by one skilled in the art by using clinical scores. A positive medical response to a drug can be readily verified in appropriate animal models of the disease which are well-known in the art.

“a”, “an”, and “the” include plural referents, unless the context clearly indicates otherwise. As such, the term “a” (or “an”), “one or more” or “at least one” can be used interchangeably herein; unless specified otherwise, “or” means “and/or”.

### Method of Identification of Functional Disease-Specific Tregs and Markers Thereof

The invention relates to a method of identification of functional disease-specific regulatory T cells, comprising the steps of:
(a) Preparing a mixture of isolated regulatory T (Treg) cells and conventional T (Tconv) cells in similar proportions from at least a patient diseased-tissue sample and a patient peripheral blood sample;
(b) Performing single-cell gene expression profiling combined with T cell receptor (TCR) profiling on each mixture of isolated Treg and Tconv cells from at least diseased-tissue and peripheral blood;
(c) Identifying clusters of Treg cells and Tconv cells, wherein the clusters comprise differentially expressed genes or gene signatures between each other; and
(d) Determining at least one cluster of functional disease-specific Treg cells among the identified clusters of Treg cells, wherein the at least one cluster comprises:
   (i) a higher proportion of Treg cells in the diseased-tissue than in the peripheral blood;
   (ii) a higher proportion of Treg cells with clonally expanded TCR specificities in the diseased-tissue; and
   (iii) a higher proportion of Treg cells with a transcriptomic signature of TCR triggering, cell activation and expansion in the diseased-tissue.

The invention also relates to a method of identification of functional disease-specific regulatory T cell markers, comprising performing steps (a) to (d) of the above method of identification of functional disease-specific regulatory T cells and performing a further step of:
(e) Identifying genes that are differentially expressed in the at least one cluster of functional disease-specific Treg cells in comparison with all the other identified clusters of Treg and Tconv cells.

The method(s) of the invention differ from the prior art method(s) in that they allow the identification of cluster(s) of functional disease-specific, in particular functional tumor-specific Tregs among the heterogeneous pool of Tregs. As a result, it is expected that the markers that are identified by the method of the invention are reliable and valid disease-specific, in particular tumor-specific, Treg markers that can be used as efficient and selective biomarker, therapeutic target or research tool. In particular, it is expected that the detection, inactivation or depletion, classification or study of functional disease-specific, in particular tumor-specific Tregs provided by the identified markers is efficient and selective and more performant than with the prior art methods.

The method is performed on at least peripheral blood samples and diseased-tissue samples, in particular tumor samples. As used herein, the term “diseased-tissue” includes diseased-tissue draining lymph node(s). Therefore, unless otherwise specified “a patient diseased-tissue sample” refers to “a patient diseased-tissue sample or a patient diseased-tissue draining lymph node sample”. As used herein, “tissue” refers to solid tissue or tissue fluid. For example, the solid tissue may be pancreatic tissue (diabetes), cartilage/joint tissue (arthritis), solid tumor tissue (cancer), and other solid tissues. Tissue fluid includes with no limitations: ascite, bronchoalveolar lavage, pleural lavage, urine, pleural fluid, cerebrospinal fluid (CSF), synovial fluid, pericardial fluid cartilage/joint fluid and peritoneal fluid. As used herein, “tumor” includes tumor tissue and tumor fluid. Tumor tissue includes: primary tumor, metastasis and tumor draining lymph node, in particular metastatic tumor draining lymph node. Tumor fluid includes all fluids draining the tumor. The method is preferably performed on both patient diseased tissue sample and patient tissue draining lymph node sample, in particular both patient tumor tissue sample and patient tumor draining lymph node sample.

The method is usually performed on samples from at least 2, preferably 3, 4, 5 or more patients. Each sample from each patient may be processed separately, i.e., the method is performed on samples from individual patients or alternatively the samples from different patients are mixed and the method is performed on a pool of patient samples. Treg and Tconv cells are isolated from peripheral blood and diseased-tissue(s) (diseased-tissue and/or draining lymph node(s)), in particular tumor(s) (tumor(s) and/or draining lymph node(s)), using standard cell isolation techniques that are well-known in the art and disclosed in the examples of the present application. Following tissue processing, Tregs and Tconvs are isolated by FACS-sorting using antibodies against specific cell-surface markers such as for example CD4, CD45, CD25 and CD127. Tregs may be defined as CD45+CD4+CD25^{hi} CD127^{lo} cells and Tconvs as CD45+CD4+CD25^{lo} CD127^{lo/hi}. In addition, the viability of the isolated cells may be measured using appropriate markers such as DAPI (viable cells are DAPI⁻). The percentage of Tregs and Tconvs in the samples is usually determined at the same time by FACS analysis. For example, FIG. 1A shows that the analysed tumor sample comprises 95.1% of Tconvs and 4.63% of Tregs. The isolated Tregs and Tconvs are then mixed in similar proportions to obtain the mixture. As used herein “in similar proportions” refers to a percentage of about 35% to about 65% (35%, 40%, 45%, 50%, 55%, 60% or 65%); preferably about 40% to about 60% (40%, 45%, 50%, 55% or 60%); more preferably of about 45% to about 55% for the Tregs and the Tconvs wherein the sum of the percentage of Tregs and the percentage of Tconvs in the mixture is equal to about 100%. The term “about” refers to a measurable value and is meant to encompass a variation of ±0.1% to 5% (0.1%; 0.5%; 1%; 1.5%; 2%; 2.5%; 3%; 3.5%; 4%; 4.5% or 5%) from the specified value. The mixture comprises at least 100 cells, usually 500 to 10000 (500, 1000, 2000, 3000, 4000, 5000, 6000, 7000, 8000, 9000 or 10000) cells or more cells including at least 100 Treg cells, preferably at least 200, 300, 400, 500, or more Tregs.

In some embodiments of the method of the invention, the patient diseased-tissue sample is patient tumor sample.

In some embodiments of the method of the invention, step (a) is further performed on patient diseased-tissue draining lymph node sample; preferably patient tumor-draining lymph node sample.

In some embodiments of the method of the invention, the isolated Tregs are CD45+CD4+CD25^{hi} CD127^{lo} cells and the isolated Tconvs are CD45+CD4+CD25^{lo} CD127^{lo/hi} cells; preferably the isolated Tregs are DAPI⁻CD45+CD4+CD25^{hi} CD127^{lo} cells and the isolated Tconvs are DAPI⁻CD45+CD4+CD25^{lo} CD127^{lo/hi} cells.

In some preferred embodiment, the mixture is composed of equal proportions of Tregs and Tconvs, which means about 50% of Tconv cells and about 50% of Treg cells.

The combined single-cell gene expression profiling and T cell receptor (TCR) profiling in step (b) is performed by standard methods that are well-known in the art and disclosed in the examples of the present application. Gene expression profiling is usually based on transcriptome analysis (transcriptome profiling), preferably by RNA sequencing technique. RNA-seq (RNA-sequencing) is a technique that can examine the quantity and sequences of RNA in a sample using next generation sequencing (NGS). It analyzes the transcriptome of gene expression patterns encoded within RNA. RNA-seq has been adapted to single-cell analysis and single-cell RNAseq was first reported by Tang et al. (Nat. Methods, 2009, 6, 377-382); review in Wang et al., Nature Reviews Genetics, 2009, 10, 57-63 and Svensson et al. (Nat Protoc. 2018 April; 13(4):599-604). TCR profiling comprises sequencing of paired TCR alpha and beta chains in individual cells to determine the final products of somatic rearrangements by V(D)J recombination, including particularly the CDR3 sequences as well as V, J, and C region usage. Transcriptome and TCR analysis can be combined using single-cell RNA-seq to identify the matched expression profile and TCR of each cell.

The identification of clusters (group of cells) of Treg cells and Tconv cells comprising differentially expressed genes or signatures in step (c) is performed by sc-RNA-seq transcriptome data analysis using bioinformatics methods that are well known in the art and disclosed in the examples of the present application. Transcriptome sequencing data by sample are processed and integrated using appropriate softwares such as Cell Ranger and Seurat. Differentially expressed genes (signatures) between clusters may be identified with FindAllMarkers function using MAST (Finak, McDavid, Yajima et al., 2015) The results of clustering may be visualized by UMAP (Uniform Manifold Approximation and Projection for Dimension Reduction; McInnes, L. and Healy, J. (2018). The clusters may comprise only Tconvs, only Tregs or may be mixed as illustrated in FIG. 2.

In some embodiments, step (c) further comprises identifying mixed clusters of Treg and Tconv cells comprising differentially expressed genes between each other.

The determination of cluster(s) of functional disease-specific Treg cells among the identified clusters of Treg cells in step (d) is performed by scTCR analysis followed by TCR expansion analysis. scTCR analysis determines the clonotypes in each tissue and analyses clonotypes between the different tissues. TCR expansion analysis measures clonal expansion by tissue. The number of cells by clonotype is determined for each tissue. When clones contain more than one cell they are considered as expanded. The percentage of expanded clones by tissue is calculated for each patient. The paired cluster obtained from scRNA-seq transcriptome analysis and TCR information allows calculation of the percentage of cells with a tumor-expanded clonotype by cluster.

Functional tumor-specific Tregs (FT-Tregs) are defined as cells that belong to a cluster (or group of cells) with all the following characteristics: (i) a cluster of CD4+ FOXP3+ Tregs: (i) that are found in the diseased tissue (in particular the tumor) or in the draining LNs (in particular metastatic tumor-draining LNs) at higher proportions than in the blood (i.e. that accumulates in tumor or in TDLN); (ii) that is enriched in cells with specificities (TCRs) that are found clonally expanded in the Treg cells from the diseased tissue (in particular tumor), and (iii) that is enriched in cells with a transcriptomic signature of recent TCR triggering, cell activation and expansion. Upon recognition of the antigens, in particular tumor antigens, via their TCR, Treg cells are activated, divide, and locally accumulate. Consequently, their transcriptome reflect these biological pathways. For example, recognition of cognate antigens via their TCR induces among others, the upregulation of genes downstream TCR activation such as REL, NKKB2, NR4A1, OX-40, 4-1BB, and known genes of Treg activation such as MHC class II molecules (HLA-DR), CD39, CD137, GITR. In some embodiments, FT-Tregs are found in the diseased tissue (in particular the tumor), and eventually also in the draining LNs (in particular tumor-draining LNs such as metastatic tumor-draining LNs) at higher proportions than in the blood or (i.e. that accumulates in tumor and eventually also in TDLNs)

In some embodiments, step (a) and step (b) are performed separately for each patient and the data from all patients obtained in step (b) are integrated to perform steps (c) to (e).

In some embodiments, the method of identification of functional disease-specific regulatory T cell markers according to the invention further comprises the identification and ranking of tumor-specific Treg markers for therapeutic purpose.

The identification and ranking of tumor-specific Treg markers for therapeutic purpose may be performed by informatics analysis, preferably comprising the following steps:
Step 1: Identifying and selecting a fraction of n differentially expressed genes which code for a cell membrane protein;
Step 2: Determining the average expression level of the n selected genes in normal tissue and assigning at least one score A to each gene from −1 for the (best) gene having the lowest expression level to −n for the (worst) gene having the highest expression level in normal tissue;
Step 3: Determining the average expression level of the n selected genes in tumoral tissue and assigning at least one score B to each gene from +n for the (best) gene having the highest expression level to +1 for the (worst) gene having the lowest expression level in tumoral tissue;
Step 4: Determining the average expression level of the n selected genes in normal PBMCs except Tregs and assigning at least one score C to each gene from +n for the (best) gene having the lowest expression level to +1 for the (worst) gene having the highest expression level in normal PBMCs except Tregs;
Step 5: Determining the average expression level of the n selected genes in the tumor environment except Tregs and assigning at least one score D to each gene from +n for the (best) gene having the lowest expression level to +1 for the (worst) gene having the highest expression level in tumor environment except Tregs;
Step 6: Determining the relative expression level of the n selected genes in i) Tumor-Tregs compared to Normal tissue-Tregs, and ii) Tregs compared to Tconvs and assigning two scores E and F to each gene from +n for the gene having the highest fold change expression level to +1 for the gene having the lowest fold change in i) (score E) Tumor Treg compared to normal adjacent tissue Treg, and ii) (score F) Tregs compared to Tconvs;
Step 7: Summating the assigned scores to obtain a cumulative assessment value (SUM SCORE) for each gene; and
Step 8: Determining the candidate therapeutic targets based on the cumulative assessment value.

The various steps of the method can be performed using well-known methods that are well-known in the art and disclosed in the present examples.

The cell-membrane protein refers to a cell-surface protein. The cell-membrane protein is preferably a transmembrane or GPI-anchored protein with an extracellular domain.

Step 1 can be performed using protein sequence annotation data available from public data bases such as Uniprot, Gene Ontology, Human protein atlas, and others, or various web tools available to determine membrane localization of protein.

Step 2 can be performed using data from gene expression profiles in healthy (normal) tissues available from public data bases such as The Genotype-Tissue Expression (GTEx) database. Immune-related tissues such as whole-blood and spleen may be deleted from healthy tissues in Step 2 as they can be better evaluated in Step 4, as disclosed in the present examples.

Step 3 can be performed using data from gene expression profiles in tumors available from public data bases such as for The Cancer Genome Atlas (TCGA) RNAseq data. Fold change of the expression level in several main cancers, in particular Lung, Breast and Colon cancer compared to normal (healthy) tissues may be used to assign a score to the n target genes.

Step 4 can be performed using data from gene expression profiles in normal PBMCs available from public data bases, preferably data from single-cell expression levels. Preferably, the functional tumor-specific Treg cluster identified in step (d) is identified in the blood, and all cells from this cluster are removed from the data sets. On the remaining cells, average expression of each target is calculated on each other cluster identified in step (c) individually and then the mean of cluster averages is calculated for each target in each dataset.

Step 5 can be performed using data from gene expression profiles in tumor environment available from public data bases, preferably data from single-cell expression levels. Data from a wide range of tumors (NSCLC, Breast cancer, PDAC, Melanoma, HCC, SCC, BCC, and others) and also a wide range of cell types (all immune cells but also tumor cells, epithelial, endothelial, cancer-associated fibroblasts and tissue-specific cell types) are advantageously used. Average expression of each target in the tumor environment may be determined as for PBMCs in Step 4.

Step 6 can be performed using data from gene expression profiles in tumor Treg and Tconv from tumor and normal adjacent tissue, for example data from bulk RNAseq. 2 scores may be determined, the fold change of expression in Treg compared to Tconv in the tumor and the fold change of expression in tumor Treg compared to Treg of normal adjacent tissue.

In Step 7 (data integration), all scores are averaged (mean) to define only one value for each parameter. The overall score of each gene is determined by summating the assigned scores (A, B, C, D and E) to obtain a cumulative assessment value (SUM SCORE) for each gene. Then, genes can be ranked by their overall score. Each target can be further characterized in term of safety (GTEx average score) and interest (SUM score of all parameters). To define cutoffs of both, a list of described activated-Treg targets can be used (IL2RA, ICOS, TNFRSF18, CCR8, CCR4, CTLA4, HAVCR2, ENTPD1, TNFRSF9). Cutoffs for both safety and interest may be set as the value of the lowest ranked reference genes.

In some embodiments, the above method of identification and ranking of tumor-specific Treg markers for therapeutic purpose, further comprises completing the profile of the potential of each gene for therapeutic targeting with information in terms of structure, function, availability of reagents, and competitive landscape. The information may be manually curated (data mining) and presented in a standardized file.

In some embodiments, the method of identification of functional disease-specific regulatory T cell markers according to the invention further comprises the steps of:
f₁) inhibiting the expression or activity or inactivating said molecular marker identified in step (e) in the functional, disease-specific, in particular tumor-specific, Tregs; and
g₁) identifying candidate therapeutic targets consisting of markers whose inhibition or inactivation modulates the viability, proliferation, stability or suppressive function of said functional, disease-specific, in particular tumor-specific Treg cells.

As used herein, “inhibiting the expression or activity of said molecular marker” includes a direct or indirect inhibition. A direct inhibition is directed specifically to the molecular marker. An indirect inhibition is directed to any effector of the molecular marker biological or signaling pathway such as with no limitations: a ligand or co-ligand, a receptor or co-receptor of said molecular marker; a co-factor or a co-effector of said molecular marker biological or signaling pathway. For example, if the molecular marker is a transcription factor or a molecule downstream a signaling cascade involving kinases, protein kinase inhibitors may be used to inhibit the molecular marker. The modulation may be an increase (stimulation) or decrease (inhibition) of the viability, proliferation or suppressive function of said tumor-specific Treg cells. An increase or stimulation of the viability, proliferation or suppressive function of said tumor-specific Treg cells indicates that the target is a Treg suppressor that should be target with an activator. A decrease or inhibition of the viability, proliferation or suppressive function of said tumor-specific Treg cells indicates that the target is a Treg activator that should be target with an inhibitor.

In some embodiments, the method according to the invention further comprises the steps of:
f₂) testing surface expression of said molecular marker identified in step (e) on the functional disease-specific, in particular tumor-specific, Tregs; and
g₂) identifying cell surface markers of functional disease-specific, in particular tumor-specific, Tregs. In some preferred embodiments, said disease is cancer. Preferably, a cancer selected from the group comprising: non-small cell lung cancer (NSCLC); breast, skin, ovarian, kidney and head and neck cancers; and rhabdoid tumors; more preferably non-small cell lung cancer (NSCLC).

In some other embodiments, said disease is chosen from acute or chronic inflammatory, allergic, autoimmune or infectious diseases, graft-versus-host disease, graft-rejection. Non-limiting examples of autoimmune diseases include: type 1 diabetes, rheumatoid arthritis, psoriasis and psoriatic arthritis, multiple sclerosis, Systemic lupus erythematosus (lupus), Inflammatory bowel disease such as Crohn's disease and ulcerative colitis, Addison's disease, Grave's disease, Sjögren's disease, alopecia areata, autoimmune thyroid disease such as Hashimoto's thyroiditis, myasthenia gravis, vasculitis including HCV-related vasculitis and systemic vasculitis, uveitis, myositis, pernicious anemia, celiac disease, Guillain-Barre Syndrome, chronic inflammatory demyelinating polyneuropathy, scleroderma, hemolytic anemia, glomerulonephritis, autoimmune encephalitis, fibromyalgia, aplastic anemia and others. Non-limiting examples of inflammatory and allergic diseases include: neuro-degenerative disorders such as Parkinson disease, chronic infections such as parasitic infection or disease like Trypanosoma cruzi infection, allergy such as asthma, atherosclerosis, chronic nephropathy, and others. The disease may be allograft rejection including transplant-rejection, graft-versus-host disease (GVHD) and spontaneous abortion

The above method of identification of functional disease-specific, in particular tumor-specific, Treg markers is also useful to classify Tregs in functional subsets and distinguishing functional-disease-specific, in particular tumor-specific, Treg clusters (FT-Tregs) out of the heterogeneous pool of Tregs. In some preferred embodiments, the disease is cancer.

### Functional Tumor-Specific Tregs and Molecular Markers Thereof

The invention also relates to the functional tumor-specific Tregs and molecular markers thereof identified by the method(s) of the invention and their various applications including in particular as biomarker, therapeutic target or research tool. The molecular biomarkers are used in particular for the detection, inactivation or depletion, classification or study of functional tumor-specific Tregs.

In particular, the invention relates to a gene signature of functional tumor-specific Tregs comprising the combination of up-regulated and down-regulated genes listed in Table 1.

The invention relates to an isolated population of functional tumor-specific Tregs having the gene signature as shown in Table 1.

The invention relates also to a molecular marker of functional tumor-specific Tregs selected from the genes of Table 1 and their RNA or protein products.

Table 1 provides a list of molecular markers of functional-tumor-specific Tregs (col. 1)); human gene ID number (col. 2); illustrative examples of accession numbers for human mRNA (col. 3) and protein sequences (col. 4 and 5) in public sequence data bases; up-regulated (+) or down-regulated gene (−) (col. 6); cell membrane status (col. 7); cell transmembrane status (col. 8) and cell surface expression (col. 9). The invention encompasses functional variants of said genes or gene products such as for example variants resulting from genetic polymorphism. The 179 genes listed in Table 1 are all up-regulated in FT-Tregs, with the exception of 4 genes: PPP2R5C, MT-ND4 (Synonym: ND4), GIMAP7, GIMAP4 which are down-regulated.

In some embodiments, the molecular marker is a cell surface marker of functional tumor-specific Tregs. Such marker is useful for the detection or targeting (activation/inactivation or depletion) of tumor-specific Tregs with antibodies or functional fragments or derivatives thereof comprising the antigen binding site.

In some preferred embodiments, the cell surface marker of functional tumor-specific Tregs is selected from the list of Table 1, said cell surface marker of functional tumor-specific Tregs being selected from the group consisting of or comprising: ADORA2A, CALR, CCR8, CD4, CD7, CD74, CD80, CD82, CD83, CSF1, CTLA4, CXCR3, HLA-B, HLA-DQA1, HLA-DR, in particular HLA-DRB5, ICAM1, ICOS, IGFLR1, IL12RB2, IL1R2, IL21R, IL2RA, IL2RB, IL2RG, LRRC32, NDFIP2, NINJ1, NTRK1, SDC4, SLC1A5, SLC3A2, SLC7A5, SLCO4A1, TMPRSS6, TNFRSF18, TNFRSF1B, TNFRSF4, TNFRSF8, TNFRSF9, TSPAN13 and TSPAN17; preferably, CCR8, CD80, ICOS, IL12RB2, CTLA-4, 4-1BB (TNFRS9), TNFRSF18 (GITR), HLA-DR, in particular HLA-DRB5, ICAM1, CSF1, CD74, OX-40 (TNFRSF4), CXCR-3, and TNFR2 (TNFRSF1B); more preferably CD74, IL12RB2, HLA-DR, in particular HLA-DRB5, ICAM1 and CSF1.

In some particular embodiments, the cell surface marker of functional tumor-specific Tregs is selected from the lists of Table 1 and Table 2, said cell surface marker of functional tumor-specific Tregs being selected from the group consisting of or comprising: CD177, CCR8, CD80, ICOS, CD39 (ENTPD1), HAVCR2 (TIM3), IL2RA, IL12RB2, CTLA-4, 4-1BB (TNFRS9), TNFRSF18 (GITR), HLA-DR, ICAM1, CSF1, CD74, OX-40 (TNFRSF4), CXCR-3, CCR4 and TNFR2 (TNFRSF1B); preferably, CD177, CCR8, CD80, ICOS, CD39, IL12RB2, CTLA-4, 4-1BB (TNFRS9), TNFRSF18 (GITR), HLA-DR, ICAM1, CSF1, CD74, OX-40 (TNFRSF4), CXCR-3, and TNFR2 (TNFRSF1B).

Cell-surface expression of the markers on Tregs can be tested by standard assays that are known in the art and disclosed in the examples of the present application, such as FACS analysis using antibodies directed to the extra-cellular domain of the marker.

In some particular embodiments, the molecular marker is selected from the group consisting of: CD177, CCR8, CD80, ICOS, CD39 (ENTPD1), HAVCR2 (TIM3), IL2RA, IL12RB2, CTLA-4, 4-1BB (TNFRS9), TNFRSF18 (GITR), HLA-DR, ICAM1, CSF1, CD74, OX-40 (TNFRSF4), CXCR-3, VDR, CCR4 and TNFR2 (TNFRSF1B); preferably, CD177, CCR8, CD80, ICOS, CD39, IL12RB2, CTLA-4, 4-1BB (TNFRS9), TNFRSF18 (GITR), HLA-DR, ICAM1, CSF1, CD74, OX-40 (TNFRSF4), CXCR-3, VDR and TNFR2 (TNFRSF1B).

In some preferred embodiments, the molecular marker is selected from the group consisting of: CCR8, CD80, ICOS, IL12RB2, CTLA-4, 4-1BB (TNFRS9), TNFRSF18 (GITR), HLA-DR, in particular HLA-DRB5, ICAM1, CSF1, CD74, OX-40 (TNFRSF4), CXCR-3, VDR and TNFR2 (TNFRSF1B); more preferably CD74, VDR, IL12RB2, HLA-DR, in particular HLA-DRB5, ICAM1 and CSF1.

In some embodiments, the marker of functional tumor-specific Tregs is a candidate therapeutic target. In particular, the marker of functional-tumor-specific Tregs modulates the viability, proliferation, destabilization and/or suppressive function of functional tumor-specific Treg cells. Such candidate therapeutic targets can be determined by standard assays that are known in the art and disclosed in the examples of the present application. Treg destabilization is disclosed in Munn et al., Cancer Res., 2018, 78, 18, 5191-5199.

For example, the candidate therapeutic targets can be selected using a method comprising the steps of:
a) inhibiting the expression or activity or inactivating said molecular marker in the functional, disease-specific, in particular tumor-specific, Tregs; and
b) identifying candidate therapeutic targets consisting of markers whose inhibition or inactivation modulates the viability, proliferation, stability or suppressive function of said functional, disease-specific, in particular tumor-specific Treg cells.

The modulation may be an increase (stimulation) or decrease (inhibition) of the viability, proliferation, suppressive function or stability of said tumor-specific Treg cells. An increase or stimulation of the viability, proliferation, stability or suppressive function of said tumor-specific Treg cells indicates that the target is a Treg suppressor that should be targeted with an activator. A decrease or inhibition of the viability, proliferation, stability or suppressive function of said tumor-specific Treg cells indicates that the target is a Treg activator that should be targeted with an inhibitor.

The markers from Table 1 which are upregulated are candidate Treg activators that should be targeted with an inhibitor. The markers from Table 1 which are downregulated are candidate Treg suppressors that should be targeted with an activator. In some preferred embodiments, the candidate therapeutic target is selected from the group comprising: CD74, Vitamin D receptor (VDR) and others; preferably CD74, VDR, IL12RB2, HLA-DR, in particular HLA-DRB5, ICAM1 and CSF1.

For example, inhibition of CD74 can be performed by blocking its co-receptor MIF with a small molecule or an anti-MIF antibody. Inhibition of VDR can be performed by inhibition of the VDR signaling pathway (beyond VDR).

In some particular embodiments, the therapeutic target is a cell surface marker of functional tumor-specific Tregs selected from the lists of Table 1 and Table 2, said therapeutic target being selected from the group consisting of or comprising: CD177, CCR8, CD80, ICOS, CD39, HAVCR2 (TIM3), IL2RA, IL12RB2, CTLA-4, 4-1BB (TNFRS9), TNFRSF18 (GITR), HLA-DR, ICAM1, CSF1, CD74, OX-40 (TNFRSF4), CXCR-3, CCR4 and TNFR2 (TNFRSF1B); preferably, CD177, CCR8, CD80, ICOS, CD39, IL12RB2, CTLA-4, 4-1BB (TNFRS9), TNFRSF18 (GITR), HLA-DR, ICAM1, CSF1, CD74, OX-40 (TNFRSF4), CXCR-3, and TNFR2 (TNFRSF1B).

In some preferred embodiments, the therapeutic target is a cell surface marker of functional tumor-specific Tregs selected from the list of Table 1, said therapeutic target being selected from the group consisting of or comprising: ADORA2A, CALR, CCR8, CD4, CD7, CD74, CD80, CD82, CD83, CSF1, CTLA4, CXCR3, HLA-B, HLA-DQA1, HLA-DR, in particular HLA-DRB5, ICAM1, ICOS, IGFLR1, IL12RB2, IL1R2, IL21R, IL2RA, IL2RB, IL2RG, LRRC32, NDFIP2, NINJ1, NTRK1, SDC4, SLC1A5, SLC3A2, SLC7A5, SLCO4A1, TMPRSS6, TNFRSF18, TNFRSF1B, TNFRSF4, TNFRSF8, TNFRSF9, TSPAN13 and TSPAN17; preferably, CCR8, CD80, ICOS, IL12RB2, CTLA-4, 4-1BB (TNFRS9), TNFRSF18 (GITR), HLA-DR, in particular HLA-DRB5, ICAM1, CSF1, CD74, OX-40 (TNFRSF4), CXCR-3, and TNFR2 (TNFRSF1B); more preferably CD74, IL12RB2, HLA-DR, in particular HLA-DRB5, ICAM1 and CSF1.

In some particular embodiments, the therapeutic target is selected from the group consisting of: CD177, CCR8, CD80, ICOS, CD39 (ENTPD1), HAVCR2 (TIM3), IL2RA, IL12RB2, CTLA-4, 4-1BB (TNFRS9), TNFRSF18 (GITR), HLA-DR, ICAM1, CSF1, CD74, OX-40 (TNFRSF4), CXCR-3, VDR, CCR4 and TNFR2 (TNFRSF1B); preferably, CD177, CCR8, CD80, ICOS, CD39, IL12RB2, CTLA-4, 4-1BB (TNFRS9), TNFRSF18 (GITR), HLA-DR, ICAM1, CSF1, CD74, OX-40 (TNFRSF4), CXCR-3, VDR and TNFR2 (TNFRSF1B).

In some preferred embodiments, the therapeutic target is selected from the group consisting of: CCR8, CD80, ICOS, IL12RB2, CTLA-4, 4-1BB (TNFRS9), TNFRSF18 (GITR), HLA-DR, in particular HLA-DRB5, ICAM1, CSF1, CD74, OX-40 (TNFRSF4), CXCR-3, VDR and TNFR2 (TNFRSF1B); more preferably CD74, VDR, IL12RB2, HLA-DR, in particular HLA-DRB5, ICAM1 and CSF1.

The present invention also encompasses a combination of markers comprising at least 2, for example 2 to 10 (2, 3, 4, 5, 6, 7, 8, 9, 10) or more markers of functional tumor-specific Tregs. In some embodiments, the combination comprises at least 2 different markers from Table 1 or Table 1 and Table 2, preferably chosen from the above listed cell-surface markers of functional tumor-specific Tregs. In some preferred embodiments, the combination comprises 2 to 10 (2, 3, 4, 5, 6, 7, 8, 9, 10) or more markers from Table 1 or Table 1 and Table 2, preferably chosen from the above listed cell-surface markers of functional tumor-specific Tregs. In some embodiments, the combination of marker is a cluster signature of a biological function, pathway, such as metabolic status, production of inhibitory cytokines or others; or cluster signature of transcription factors and upstream regulators.

### Diagnosis, Prognosis, Monitoring of Cancer

Tregs actively suppress anti-tumor immune responses and elevated frequencies of Tregs are found in many human cancers and are associated with poor clinical outcomes. Therefore, the functional tumor-specific Tregs and markers thereof according to the invention, including the combinations of said markers are useful as biomarkers for the diagnosis, prognosis and monitoring of cancer.

Therefore, the invention relates to the in vitro use of functional tumor-specific Tregs or markers or combination of markers thereof according to the present disclosure as a biomarker for the diagnosis, prognosis and monitoring of cancer.

The invention also relates to an in vitro method of diagnosis, prognosis or monitoring of cancer, comprising the step of detecting the presence of functional tumor-specific Tregs according to the present disclosure, in a tumor sample from a subject. The detection may be performed according to step (a) to (d) of the method of identification of FT-Tregs according to the present disclosure. The detection may be semi-quantitative or quantitative and may comprise detection of the presence or level of functional tumor-specific Tregs.

The invention also relates to an in vitro method of diagnosis, prognosis or monitoring of cancer, comprising the step of detecting the expression of at least one marker of functional tumor-specific Tregs according to the present disclosure, in a tumor sample from a subject.

In some embodiments, the molecular marker of functional tumor-specific Tregs is selected from the genes of Table 1 and their RNA or protein products.

In some particular embodiments, the molecular marker is a cell surface marker of functional tumor-specific Tregs selected from the lists of Table 1 and Table 2, said therapeutic target being selected from the group consisting of or comprising: CD177, CCR8, CD80, ICOS, CD39, HAVCR2 (TIM3), IL2RA, IL12RB2, CTLA-4, 4-1BB (TNFRS9), TNFRSF18 (GITR), HLA-DR, ICAM1, CSF1, CD74, OX-40 (TNFRSF4), CXCR-3, CCR4 and TNFR2 (TNFRSF1B); preferably, CD177, CCR8, CD80, ICOS, CD39, IL12RB2, CTLA-4, 4-1BB (TNFRS9), TNFRSF18 (GITR), HLA-DR, ICAM1, CSF1, CD74, OX-40 (TNFRSF4), CXCR-3, and TNFR2 (TNFRSF1B).

In some particular embodiments, the molecular is a cell surface marker of functional tumor-specific Tregs selected from the list of Table 1, said therapeutic target being selected from the group consisting of or comprising: ADORA2A, CALR, CCR8, CD4, CD7, CD74, CD80, CD82, CD83, CSF1, CTLA4, CXCR3, HLA-B, HLA-DQA1, HLA-DR such as HLA-DRB5, ICAM1, ICOS, IGFLR1, IL12RB2, IL1R2, IL21R, IL2RA, IL2RB, IL2RG, LRRC32, NDFIP2, NINJ1, NTRK1, SDC4, SLC1A5, SLC3A2, SLC7A5, SLCO4A1, TMPRSS6, TNFRSF18, TNFRSF1B, TNFRSF4, TNFRSF8, TNFRSF9, TSPAN13 and TSPAN17; preferably, CCR8, CD80, ICOS, IL12RB2, CTLA-4, 4-1BB (TNFRS9), TNFRSF18 (GITR), HLA-DR such as HLA-DRB5, ICAM1, CSF1, CD74, OX-40 (TNFRSF4), CXCR-3, and TNFR2 (TNFRSF1B); more preferably CD74, IL12RB2, HLA-DR such as HLA-DRB5, ICAM1 and CSF1.

In some particular embodiments, the molecular marker is selected from the group consisting of: CD177, CCR8, CD80, ICOS, CD39 (ENTPD1), HAVCR2 (TIM3), IL2RA, IL12RB2, CTLA-4, 4-1BB (TNFRS9), TNFRSF18 (GITR), HLA-DR, ICAM1, CSF1, CD74, OX-40 (TNFRSF4), CXCR-3, VDR, CCR4 and TNFR2 (TNFRSF1B); preferably, CD177, CCR8, CD80, ICOS, CD39, IL12RB2, CTLA-4, 4-1BB (TNFRS9), TNFRSF18 (GITR), HLA-DR, ICAM1, CSF1, CD74, OX-40 (TNFRSF4), CXCR-3, VDR and TNFR2 (TNFRSF1B).

In some preferred embodiments, the molecular marker is selected from the group consisting of: CCR8, CD80, ICOS, IL12RB2, CTLA-4, 4-1BB (TNFRS9), TNFRSF18 (GITR), HLA-DR, in particular HLA-DRB5, ICAM1, CSF1, CD74, OX-40 (TNFRSF4), CXCR-3, VDR and TNFR2 (TNFRSF1B); more preferably CD74, VDR, IL12RB2, HLA-DR, in particular HLA-DRB5, ICAM1 and CSF1.

In some embodiments, the method comprises the detection of a combination of at least 2 different markers from Table 1. In some particular embodiments, the combination of at least 2 different markers from Table 1 comprises at least one molecular from Table 1 or Table 1 and Table 2, as listed above, preferably at least one cell surface marker as listed above.

In some embodiments, the molecular marker is detected in a subset of FT-Tregs identified according to step (a) to (d) of the method of identification of FT-Tregs according to the present disclosure.

The detection may be semi-quantitative or quantitative and may comprise detection of the presence or level of expression of the marker. The detection may be performed on the whole tumor or on a fraction of isolated cells comprising or consisting of Tregs. The expression may be determined at the RNA of protein level. The level of expression may refer to the amount of marker RNA or protein or the number of cells expressing said RNA or protein. The level of expression in the test sample to analyse is compared with a predetermined value or with the value obtained with a control sample tested in parallel. Typically, the expression level in a patient sample is deemed to be higher or lower than the predetermined value obtained from the general population or from healthy subjects if the ratio of the expression level of said marker in said patient to that of said predetermined value is higher or lower than 1.2, preferably 1.5, even more preferably 2, even more preferably 5, 10 or 20.

As used herein, the term “predetermined value of a marker” refers to the amount of the marker in biological samples obtained from the general population or from a selected population of subjects. For example, the general population may comprise apparently healthy subjects, such as individuals who have not previously had any sign or symptoms indicating the presence of cancer. The term “healthy subjects” as used herein refers to a population of subjects who do not suffer from any known condition, and in particular who are not affected with any cancer. In another example, the predetermined value may be the amount of marker obtained from selected population of subjects having an established cancer but who shows a clinically significant relief in a cancer type when treated with a cancer drug. The predetermined value can be a threshold value, or a range. The predetermined value can be established based upon comparative measurements between apparently healthy subjects and subjects with established cancer.

The expression of said marker may be determined by any suitable methods known by skilled persons. Usually, these methods comprise measuring the quantity of mRNA or protein. Methods for determining the quantity of mRNA are well known in the art. For example, the mRNA contained in the sample is first extracted according to standard methods, for example using lytic enzymes or chemical solutions or extracted by nucleic-acid-binding resins following the manufacturer's instructions. The extracted mRNA is then detected by hybridization (e.g., Northern blot analysis) and/or amplification (e.g., RT-PCR). Quantitative or semi-quantitative RT-PCR is preferred. In a preferred embodiment, the mRNA expression level is measured by RNA seq method, more preferably by single-cell RNA-seq. RNA seq can be used to analyse the cellular transcriptome. RNAseq, preferably single cell RNA seq can be performed for example in plate, micro or nano-wells, droplet-based microfluidics, microfluidics, tubes as disclosed in the examples of the present application.

Protein expression may be determined by any suitable methods known by skilled persons. Usually, these methods comprise contacting a cell sample, preferably a cell lysate, with a binding partner capable of selectively interacting with the protein present in the sample. The binding partner is generally a polyclonal or monoclonal antibodies, preferably monoclonal. The quantity of the protein may be measured, for example, by semi-quantitative Western blots, enzyme-labelled and mediated immunoassays, such as ELISAs, biotin/avidin type assays, radioimmunoassay, immune-electrophoresis or immunoprecipitation or by protein or antibody arrays. The reactions generally include revealing labels such as fluorescent, chemiluminescent, radioactive, enzymatic labels or dye molecules, or other methods for detecting the formation of a complex between the antigen and the antibody or antibodies reacted therewith.

In some embodiments of the above methods of diagnosis, prognosis or monitoring of cancer, the detection step is further performed on tumor draining lymph node(s) sample and/or blood sample from the subject. The blood sample may serve as control.

In some embodiments, the method comprises detecting the level of expression of the marker in the tumor sample, and eventually also in tumor draining lymph node(s) sample and/or blood sample from the subject.

The presence or level of the marker(s) in the patient sample is indicative of an unfavourable outcome of the cancer in the patient before undergoing cancer treatment or in the course of cancer treatment. An unfavourable outcome includes one or more of a reduced survival time, an increased tumor evolution, an increased metastasis, or an increased recurrence of the cancer in the patient.

In some embodiments, the method comprises the further step of determining from the presence, absence or level of expression of said marker whether the outcome of the cancer in the patient is favorable or unfavorable.

In some embodiments, the method comprises the further step of classifying the patient into favorable or unfavorable outcome category based on the presence, absence or level of expression of said marker of functional tumor-specific Treg in the patient tumor sample.

This step improves the treatment by determining the patients who are at risk of unfavourable outcome and should benefit from a more aggressive or targeted therapy.

In some embodiments, the marker is a therapeutic target or a combination of therapeutic targets, in particular selected from the therapeutic targets listed in Table 1 or Table 1 and Table 2; more preferably from the cell-surface markers of Table 1 or Table 1 and Table 2 as listed above. In this embodiment, the presence or level of the marker(s) in the patient sample is indicative that the patient is a responder to therapy targeting said therapeutic target. This method improves the efficiency of cancer treatment by determining the patients who are likely to be responders to the treatment before administration of said treatment.

As used herein, the term “cancer” refers to any cancer that may affect any one of the following tissues or organs: breast; liver; kidney; heart, mediastinum, pleura; floor of mouth; lip; salivary glands; tongue; gums; oral cavity; palate; tonsil; larynx; trachea; bronchus, lung; pharynx, hypopharynx, oropharynx, nasopharynx; esophagus; digestive organs such as stomach, intrahepatic bile ducts, biliary tract, pancreas, small intestine, colon; rectum; urinary organs such as bladder, gallbladder, ureter; rectosigmoid junction; anus, anal canal; skin; bone; joints, articular cartilage of limbs; eye and adnexa; brain; peripheral nerves, autonomic nervous system; spinal cord, cranial nerves, meninges; and various parts of the central nervous system; connective, subcutaneous and other soft tissues; retroperitoneum, peritoneum; adrenal gland; thyroid gland; endocrine glands and related structures; female genital organs such as ovary, uterus, cervix uteri; corpus uteri, vagina, vulva; male genital organs such as penis, testis and prostate gland; hematopoietic and reticuloendothelial systems; blood; lymph nodes; thymus.

The term “cancer” according to the invention comprises leukemias, seminomas, melanomas, teratomas, lymphomas, non-Hodgkin lymphoma, neuroblastomas, gliomas, adenocarcinoma, mesothelioma (including pleural mesothelioma, peritoneal mesothelioma, pericardial mesothelioma and end stage mesothelioma), rectal cancer, endometrial cancer, thyroid cancer (including papillary thyroid carcinoma, follicular thyroid carcinoma, medullary thyroid carcinoma, undifferentiated thyroid cancer, multiple endocrine neoplasia type 2A, multiple endocrine neoplasia type 2B, familial medullary thyroid cancer, pheochromocytoma and paraganglioma), skin cancer (including malignant melanoma, basal cell carcinoma, squamous cell carcinoma, Kaposi's sarcoma, keratoacanthoma, moles, dysplastic nevi, lipoma, angioma and dermatofibroma), nervous system cancer, brain cancer (including astrocytoma, medulloblastoma, glioma, lower grade glioma, ependymoma, germinoma (pinealoma), glioblastoma multiform, oligodendroglioma, schwannoma, retinoblastoma, congenital tumors, spinal cord neurofibroma, glioma or sarcoma), skull cancer (including osteoma, hemangioma, granuloma, xanthoma or osteitis deformans), meninges cancer (including meningioma, meningiosarcoma or gliomatosis), head and neck cancer (including head and neck squamous cell carcinoma and oral cancer (such as, e.g., buccal cavity cancer, lip cancer, tongue cancer, mouth cancer or pharynx cancer)), lymph node cancer, gastrointestinal cancer, liver cancer (including hepatoma, hepatocellular carcinoma, cholangiocarcinoma, hepatoblastoma, angiosarcoma, hepatocellular adenoma and hemangioma), colon cancer, stomach or gastric cancer, esophageal cancer (including squamous cell carcinoma, larynx, adenocarcinoma, leiomyosarcoma or lymphoma), colorectal cancer, intestinal cancer, small bowel or small intestines cancer (such as, e.g., adenocarcinoma lymphoma, carcinoid tumors, Kaposi's sarcoma, leiomyoma, hemangioma, lipoma, neurofibroma or fibroma), large bowel or large intestines cancer (such as, e.g., adenocarcinoma, tubular adenoma, villous adenoma, hamartoma or leiomyoma), pancreatic cancer (including ductal adenocarcinoma, insulinoma, glucagonoma, gastrinoma, carcinoid tumors or vipoma), ear, nose and throat (ENT) cancer, breast cancer (including HER2-enriched breast cancer, luminal A breast cancer, luminal B breast cancer and triple negative breast cancer), cancer of the uterus (including endometrial cancer such as endometrial carcinomas, endometrial stromal sarcomas and malignant mixed Müllerian tumors, uterine sarcomas, leiomyosarcomas and gestational trophoblastic disease), ovarian cancer (including dysgerminoma, granulosa-theca cell tumors and Sertoli-Leydig cell tumors), cervical cancer, vaginal cancer (including squamous-cell vaginal carcinoma, vaginal adenocarcinoma, clear cell vaginal adenocarcinoma, vaginal germ cell tumors, vaginal sarcoma botryoides and vaginal melanoma), vulvar cancer (including squamous cell vulvar carcinoma, verrucous vulvar carcinoma, vulvar melanoma, basal cell vulvar carcinoma, Bartholin gland carcinoma, vulvar adenocarcinoma and erythroplasia of Queyrat), genitourinary tract cancer, kidney cancer (including clear renal cell carcinoma, chromophobe renal cell carcinoma, papillary renal cell carcinoma, adenocarcinoma, Wilms tumor, nephroblastoma, lymphoma or leukemia), adrenal cancer, bladder cancer, urethra cancer (such as, e.g., squamous cell carcinoma, transitional cell carcinoma or adenocarcinoma), prostate cancer (such as, e.g., adenocarcinoma or sarcoma) and testis cancer (such as, e.g., seminoma, teratoma, embryonal carcinoma, teratocarcinoma, choriocarcinoma, sarcoma, interstitial cell carcinoma, fibroma, fibroadenoma, adenomatoid tumors or lipoma), lung cancer (including small cell lung carcinoma (SCLC), non-small cell lung carcinoma (NSCLC) including squamous cell lung carcinoma, lung adenocarcinoma (LUAD), and large cell lung carcinoma, bronchogenic carcinoma, alveolar carcinoma, bronchiolar carcinoma, bronchial adenoma, lung sarcoma, chondromatous hamartoma and pleural mesothelioma), sarcomas (including Askin's tumor, sarcoma botryoides, chondrosarcoma, Ewing's sarcoma, malignant hemangioendothelioma, malignant schwannoma, osteosarcoma and soft tissue sarcomas), soft tissue sarcomas (including alveolar soft part sarcoma, angiosarcoma, cystosarcoma phyllodes, dermatofibrosarcoma protuberans, desmoid tumor, desmoplastic small round cell tumor, epithelioid sarcoma, extraskeletal chondrosarcoma, extraskeletal osteosarcoma, fibrosarcoma, gastrointestinal stromal tumor (GIST), hemangiopericytoma, hemangiosarcoma, Kaposi's sarcoma, leiomyosarcoma, liposarcoma, lymphangiosarcoma, lymphosarcoma, malignant peripheral nerve sheath tumor (MPNST), neurofibrosarcoma, plexiform fibrohistiocytic tumor, rhabdomyosarcoma, synovial sarcoma and undifferentiated pleomorphic sarcoma, cardiac cancer (including sarcoma such as, e.g., angiosarcoma, fibrosarcoma, rhabdomyosarcoma or liposarcoma, myxoma, rhabdomyoma, fibroma, lipoma and teratoma), bone cancer (including osteogenic sarcoma, osteosarcoma, fibrosarcoma, malignant fibrous histiocytoma, chondrosarcoma, Ewing's sarcoma, malignant lymphoma and reticulum cell sarcoma, multiple myeloma, malignant giant cell tumor chordoma, osteochronfroma, osteocartilaginous exostoses, benign chondroma, chondroblastoma, chondromyxoid fibroma, osteoid osteoma and giant cell tumors), hematologic and lymphoid cancer, blood cancer (including acute myeloid leukemia, chronic myeloid leukemia, acute lymphoblastic leukemia, chronic lymphocytic leukemia, myeloproliferative diseases, multiple myeloma and myelodysplasia syndrome), Hodgkin's disease, non-Hodgkin's lymphoma and hairy cell and lymphoid disorders, and the metastases thereof.

In some embodiments the cancer is selected from the group comprising: non-small cell lung cancer (NSCLC); breast, skin, ovarian, kidney and head and neck cancers; and rhabdoid tumors; preferably non-small cell lung cancer (NSCLC).

### Cancer Treatment

Tregs actively suppress anti-tumor immune responses and depleting/inactivating Tregs has proven very valuable to increase anti-tumor responses. Therefore, markers of functional tumor-specific Tregs according to the present disclosure which are candidate therapeutic targets are useful for the development of new anti-cancer agents and cancer therapies including for example approaches based on cell-therapy including adoptive cell therapy, on antibodies, cytokines or chemical drugs that induce selective depletion or functional alteration of Treg cells. Selective inhibition of tumor-specific Tregs, while preserving effector T cells and Tregs from healthy tissues (that maintain immune homeostasis and control autoimmunity), represents a more effective and safer strategy that should lead to the enhancement of effective anti-tumor immunity, without eliciting generalized autoimmunity.

Therefore, the invention relates to an agent or a combination of agents for use as a Treg-inactivating or Treg-depleting agent in a method of treating cancer.

In some embodiment, said agent is a modulator of a therapeutic target according to the present disclosure which is used to inactive Tregs.

In some embodiments, the therapeutic target is selected from the genes of Table 1 or Table 1 and Table 2, and their RNA or protein products. In some particular embodiments, the therapeutic target is selected from the cell-surface markers of Table 1 or Table 1 and Table 2 as listed above, and their RNA or protein products. In some preferred embodiments, the therapeutic target is selected from the group comprising: CD74, Vitamin D receptor (VDR) and others; more preferably CD74, VDR, IL12RB2, HLA-DR, in particular HLA-DRB5, ICAM1 and CSF1.

In some embodiments, the combination of agents comprises a combination of modulators of therapeutic targets which targets at least 2 different genes from Table 1 or Table 1 and Table 2, including their RNA or protein products. In some particular embodiments, the combination targets at least one cell-surface marker of Table 1 or Table 1 and Table 2 as listed above, and their RNA or protein products.

The modulator may inhibit or stimulate the activity or expression of the therapeutic target. As used herein, “inhibiting or stimulating the expression or activity of said molecular marker” includes a direct or indirect inhibition or stimulation. A direct inhibition or stimulation is directed specifically to the molecular marker. An indirect inhibition or stimulation is directed to any effector of the molecular marker biological or signaling pathway such as with no limitations: a ligand or co-ligand, a receptor or co-receptor of said molecular marker; a co-factor or a co-effector of said molecular marker biological or signaling pathway. For example, inhibition of CD74 function as MIF co-receptor can be performed by using a small molecule or an anti-MIF antibody. Inhibition of VDR can be performed by inhibition of the VDR signaling pathway (beyond VDR).

The modulator inhibits or decreases the viability, proliferation, stability and/or suppressive function of (functional) tumor-specific Treg cells. The inhibiting or stimulating activity of an agent on the expression or activity of a therapeutic target or its inhibiting or decreasing activity on the viability, proliferation, stability and/or suppressive function of (functional) tumor-specific Treg cells may be tested by standard assays that are known in the art and disclosed in the examples of the present application.

In some preferred embodiments, the modulator inhibits or stimulates the activity of the therapeutic target. The modulator of activity may be selected from the group comprising: small organic molecules, aptamers, antibodies, and other agonists or antagonists such as for example dominant negative mutants or functional fragments of the therapeutic target protein.

The term “small organic molecule” refers to a molecule of a size comparable to those of organic molecules generally used in pharmaceuticals. The term excludes biological macro molecules (e.g., proteins, nucleic acids, etc.). Preferred small organic molecules range in size up to about 5000 Da, more preferably up to 2000 Da, and most preferably up to about 1000 Da. Various small organic molecule inhibitors or antagonists are known in the art. Identification of new small molecule inhibitors can be achieved according to classical techniques in the field. The current prevailing approach to identify hit compounds is through the use of a high throughput screen (HTS).

Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by Exponential enrichment (SELEX) of a random sequence library, as described in Tuerk C. and Gold L., 1990 and can be optionally chemically modified.

As used herein, the term “antibody” refers to a protein that includes at least one antigen-binding region of immunoglobulin. The antigen binding region may comprise one or two variable domains, such as for example a VH domain and a VL domain or a single VHH or VNAR domain. The term “antibody” encompasses full length immunoglobulins of any isotype, functional fragments thereof comprising at least the antigen-binding region and derivatives thereof. Antigen-binding fragments of antibodies include for example Fv, scFv, Fab, Fab′, F(ab′)2, Fd, Fabc and sdAb (V_{H}H, V-NAR). Antibody derivatives include with no limitation polyspecific or multivalent antibodies, intrabodies and immunoconjugates. Intrabodies are antibodies that bind intracellularly to their antigen after being produced in the same cell (for a review see for example, Marschall A L, Dube'S and Boldicke T “Specific in vivo knockdown of protein function by intrabodies”, MAbs. 2015; 7(6):1010-35). The antibody may be glycosylated. An antibody can be functional for antibody-dependent cytotoxicity and/or complement-mediated cytotoxicity, or may be non-functional for one or both of these activities. Antibodies are prepared by standard methods that are well-known in the art such as hybridoma technology, selected lymphocyte antibody method (SLAM), transgenic animals, recombinant antibody libraries or synthetic production.

In some particular embodiments, the modulator inhibits the activity of the therapeutic target.

In some other particular embodiments, the modulator inhibits the expression of the therapeutic target. In some preferred embodiments, the inhibitor is selected from the group comprising: anti-sense oligonucleotides, interfering RNA molecules, ribozymes and genome or epigenome editing systems.

Anti-sense oligonucleotides are RNA, DNA or mixed and may be modified. Interfering RNA molecules include with no limitations siRNA, shRNA and miRNA. Genome and Epigenome editing system may be based on any known system such as CRISPR/Cas, TALENs, Zinc-Finger nucleases and meganucleases. Anti-sense oligonucleotides, interfering RNA molecules, ribozymes, genome and epigenome editing systems are well-known in the art and inhibitors of the therapeutic target according to the invention may be easily designed based on these technologies using the sequences of the therapeutic targets that are well-known in the art.

In some other embodiments, the agent comprises a molecule which binds to a cell surface marker of functional tumor-specific Tregs according to the present disclosure and a compound which inactivates or destabilizes Tregs, which is used to inactivate Tregs.

The molecule which binds to said cell surface marker of functional tumor-specific Tregs is preferably an antibody or a functional fragment thereof comprising the antigen binding site. The antibody is directed to the extracellular domain of the cell surface marker of functional tumor-specific Tregs.

Compounds which inactivate or destabilize Tregs are well-known in the art and include with no limitations chemical drugs modulating Treg-associated pathways, like cyclophosphamide (Lutsiak et al., Blood, 2005, 105, 2862-2868), fludarabine, gemcitabine, and mitoxantrone (Dwarakanath et al., Cancer Rep., 2018, 1, e21105; Wang et al., Cell Rep., 2018, 23, 3262-3274); Treg-depleting antibodies (like anti-CTLA-4, anti-CD25, anti-CCR5, anti-CCR4; Dwarakanath et al., Cancer Rep., 2018, 1, e21105); Cytokines and modified cytokines including for example high dose IL-2 (to stimulate effector cells in cancer), and IL-2-derivatives with specific selectivity to Tregs or effector cells (IL-2/anti-IL-2 complexes, pegylated IL-2; resurfaced IL-2 variants (Pero′, L., Piaggio, E., 2016. New Molecular and Cellular Mechanisms of Tolerance: Tolerogenic Actions of IL-2, in: Cuturi, M. C., Anegon, I. (Eds.), Suppression and Regulation of Immune Responses. Springer New York, N.Y., N.Y., pp. 11-28).

The agent may be an immunoconjugate, a bispecific antibody or an antibody fused to a protein compound which inhibits Tregs such as a cytokine or modified cytokine including for example IL-2 and IL-2-derivative with specific selectivity to Tregs or effector cells (IL-2/anti-IL-2 complexes, resurfaced IL-2 variants).

In some other embodiments, the agent is a cytotoxic agent comprising a molecule which binds to a cell surface marker of functional tumor-specific Tregs according to the present disclosure and a cytotoxic compound, which is used to deplete Tregs.

The molecule which binds to said cell surface marker of functional tumor-specific Tregs is preferably an antibody or a functional fragment thereof comprising the antigen binding site. The antibody is directed to the extracellular domain of the cell surface marker of functional tumor-specific Tregs. The cytotoxic compound is any cytotoxic compound that is used in immunotoxin such as toxins, antibiotics, radioactive isotopes and nucleolytic enzymes.

In some other embodiments, the agent is a cytotoxic antibody directed to a cell surface marker of functional tumor-specific Tregs according to the present disclosure, which is used to deplete Tregs. The cytotoxic antibody may have CDC or ADCC activity.

In some embodiments, the agent is delivered by a recombinant vector. Recombinant vectors include usual vectors used in genetic engineering and gene therapy including for example plasmids and viral vectors.

The agent may be used to inactivate or deplete tumor-specific Treg cells in vivo or ex vivo (cell-based therapy). Cell-based therapy comprises the preparation of tumor-infiltrating lymphocytes (TILs) from a patient tumor biopsy using standard methods which are well-known in the art. The TILs are usually expanded in vitro before treatment with the agent according to the invention which inactivates or depletes functional tumor-specific Tregs present in the patient tumor. After treatment, the TILs are re-injected to the patient.

The invention also encompasses an engineered Treg cell defective for at least one of the up-regulated genes of Table 1 or Table 1 and Table 2, or which over-expresses at least one of the down-regulated genes of Table 1 or Table 1 and Table 2, in particular at least one of the cell-surface markers of Table 1 or Table 1 and Table 2 as listed above. The genetic modification of Tregs according to the present disclosure lead to the enhancement of effective anti-tumor immunity, without eliciting generalized autoimmunity.

In some embodiments, the engineered Treg cell further comprises at least one genetically engineered antigen receptor that specifically binds a target antigen. The target antigen is preferably expressed in cancer cells and/or is a universal tumor antigen. The genetically engineered antigen receptor is preferably a chimeric antigen receptor (CAR) or a T cell receptor (TCR).

The invention also relates to a method of producing an engineered Treg cell according to the present disclosure comprising the step of disrupting at least one of the up-regulated genes of Table 1 or Table 1 and Table 2, in the Treg cell or introducing the down-regulated gene of Table 1 or Table 1 and Table 2, in particular at least one cell-surface markers of Table 1 or Table 1 and Table 2 as listed above, or a functional construct thereof in the Treg cell. Preferably, the method further comprises a step of introducing into said Treg cell a genetically engineered antigen receptor that specifically binds to a target antigen. The method is performed by standard knock-in and knock-out techniques, preferably using gene editing systems such as CRISPR/Cas, TALEN and meganucleases.

In some embodiments, the Treg cell is a tumor-specific Treg cell which may be an autologous Treg cell or an allogeneic Treg cell. The Treg cell is preferably a functional tumor-specific Treg according to the present disclosure. The FT-Treg is isolated from a patient tumor biopsy.

The invention further relates to the engineered Treg cell according to the present disclosure or obtained according to the method of the present disclosure, or a pharmaceutical composition or a kit comprising said engineered Treg cell, for use in adoptive cellular therapy of cancer.

The agent or engineered Treg is advantageously used in the form of a pharmaceutical composition comprising, as active substance the agent, vector or engineered Treg according to the invention, and at least one pharmaceutically acceptable vehicle and/or carrier.

The pharmaceutical composition is formulated for administration by a number of routes, including but not limited to oral, parenteral and local. The pharmaceutical vehicles are those appropriate to the planned route of administration, which are well known in the art.

The pharmaceutical composition comprises a therapeutically effective amount of agent, vector or engineered Treg sufficient to show a positive medical response in the individual to whom it is administered. A positive medical response refers to the reduction of subsequent (preventive treatment) or established (therapeutic treatment) disease symptoms. The positive medical response comprises a partial or total inhibition of the symptoms of the disease. A positive medical response can be determined by measuring various objective parameters or criteria such as objective clinical signs of the disease and/or the increase of survival. A medical response to the composition according to the invention can be readily verified in appropriate animal models of the disease which are well-known in the art.

The pharmaceutically effective dose depends upon the composition used, the route of administration, the type of mammal (human or animal) being treated, the physical characteristics of the specific mammal under consideration, concurrent medication, and other factors, that those skilled in the medical arts will recognize.

By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

The pharmaceutical composition of the present invention is generally administered according to known procedures, at dosages and for periods of time effective to induce a beneficial effect in the individual. The administration may be by injection or by oral, sublingual, intranasal, rectal or vaginal administration, inhalation, or transdermal application. The injection may be subcutaneous, intramuscular, intravenous, intraperitoneal, intradermal or else.

In some embodiments, the pharmaceutical composition comprises another active agent such as in particular an immunomodulatory agent, an anticancer or a tumor antigen.

The pharmaceutical composition of the invention is advantageously used in combination with additional cancer therapies such as with no limitations: immunotherapy including immune checkpoint therapy and immune checkpoint inhibitor, co-stimulatory antibodies, CAR-T cell therapy, anticancer vaccine; chemotherapy and/or radiotherapy. The combined therapies may be separate, simultaneous, and/or sequential.

In some preferred embodiments the cancer is selected from the group comprising: non-small cell lung cancer (NSCLC); breast, skin, ovarian, kidney and head and neck cancers; and rhabdoid tumors; more preferably non-small cell lung cancer (NSCLC).

In some embodiments, the pharmaceutical composition is used for the treatment of humans.

In some embodiments, the pharmaceutical composition is used for the treatment of animals.

The practice of the present invention will employ, unless otherwise indicated, conventional techniques which are within the skill of the art. Such techniques are explained fully in the literature.

The invention will now be exemplified with the following examples, which are not limitative, with reference to the attached drawings in which:

### FIGURE LEGENDS

FIG. 1: Data Description
A. 5000 Tconvs (DAPI− CD45+CD4+CD2510 CD127lo/hi) and 5000 Tregs (DAPI− CD45+CD4+CD25hi CD127lo) were FACS sorted and admixed in equal numbers for scRNAseq analysis using 10× Genomics. B. Number of total cells recovered in each patient and tissue. C. Samples were analyzed using Cell Ranger V3 and Seurat 2 pipeline and shown is the UMAP visualization of all cells aggregated after CCA batch-effect correction. A resolution of 0.9 (Louvain algorithm) was used which defined 21 clusters (visualized with different colors). Upper and lower zones delimited by dotted-lines include Tregs and Tconvs, respectively, and were defined using heatmap of differentially expressed genes, gene and signature expression (see examples in FIG. 2).

FIG. 2: Identification of T Cell Clusters
T cell clusters were defined by UMAP projection of selected genes (“features”) or signatures extracted from the literature. A. Panels show how CD4+ T cony cells were identified as expressing CD40L, and CD127: and Tregs were identified as expressing FOXP3, CD25, and expressing genes of a published Treg signature (* Zemmour et al., 2018 and ** Azizi et al, 2018). B. Panels show how CD4+ T cells showing a naïve phenotype were identified using the published signature in Stubbington et al., 2015; terminally differentiated cells were identified using the published signature in Azizi et al, 2018; central memory cells were identified as in Abbas A R et al., 2009, cycling cells as in Chung et al., 2017, cells with an IFN alpha response signature were identified as in MSigDB (HALLMARK_INTERFERON_ALPHA_RESPONSE, M5911), T follicular helper cells as in Kenefeck R et al; 2015, and Th17 cells as in Zhang W et al; 2012. C. Panels show the final cluster classification of T cells: a total of 7 pure Tconv cell clusters were identified (Tconv clusters 1-7), a total of 5 pure Treg clusters were identified (Treg clusters 1-5) and a total of 9 «mixed T cell» clusters were identified, which were composed of mixtures of cells with Treg and Tconv characteristics (Tmix 1-9).

FIG. 3: Identification of Treg Clusters that Accumulate in Tumor or LNs, Compared to the Blood
Comparison of the percentages of total Tregs of each of the 5 Treg clusters among the 3 tissues. Only the proportions of Treg clusters 4 and 5 are statistically significantly increased in TDLNs or tumors, compared with the blood (paired-t test <0.05).

FIG. 4: Identification of Tregs Bearing TCRs that are Clonally Expanded in the Tumor
A. Table with clonotype information for all patients. B-D. Results are shown for Patient 4. B. Distribution of total and expanded clones for patient 4. C. Shown are the % of clones with TCR found expanded by location (blood, tumor draining lymph nodes and tumor) (Left panel) and the % of clones with TCR found expanded in the tumor for each Treg cluster (Right panel). D. UMAP projection of cells expressing TCRs found expanded in tumor cells. Each dot is a cell. From left to right are highlighted the expanded clones in Blood, TDLN, Tumor and all tissues together.

FIG. 5: Identification of Clusters of CD4+ FOXP+ Tregs with Transcriptomic Signatures of TCR Triggering, Cell Activation and Expansion
UMAP projection of cells expressing selected signature or genes (darker dots). TCR activation signature was extracted from MSigDB (REACTOME_DOWNSTREAM_TCR_SIGNALING, MI3166).

FIG. 6: Differential Expressed Gene Analysis (DEG)
DEGs analysis of cells with tumor-expanded clonotypes and present in the Treg cluster 4 (from all the patients together) versus the cells belonging to individual clusters (Treg 1-5; Tconv 1-7, Tmix 1-7) were intersected. The genes always up-regulated or always down-regulated were considered as the tumor-specific Treg features.

FIG. 7: Identification of Selected Markers of Tumor-Specific Tregs
UMAP projection of cells expressing some selected genes (darker dots).

FIG. 8: Graphical Summary of the Developed Pipeline

FIG. 9: Selection Pipeline Output.
Each dot represents a target. Targets are ranked by their gene rank (final score of the selection pipeline) and plotted against their GTEx safety score. In red are indicated the known Treg reference genes. ENTPD1 (CD39) was the lowest ranked Treg reference for safety and score hence chosen for both cutoffs.

FIG. 10: Representative FACS dot plots showing the expression of model candidate tumor-specific Treg marker CCR8 on Treg cells (gated as CD4+ FOXP3 T cells), obtained from blood (PBMC), tumor-draining lymph node (TDLN) and tumor from a NSCLC patient.
Numbers in the gates represent the percentage of Tregs positive for CCR8.

FIG. 11: Representative Dot Plots Depicting the Expression of Selected Genes in Matched CD4+ T Cells from PBMCs and Tumors from Free-of-Treatment NSCLC Patients.
A. Representative dot plots depicting the expression of FOXP3+ among CD4+CD3+ live cells from PBMCs and tumor of the same patients (numbers indicate percentages of cells in the indicated gates), and B. Level of expression (MFI) of CD4, FOXP3 and CD25 in the Treg and Tconv populations from the 2 analyzed tissues (numbers are the MFI values). Representative dot plots depicting the expression of CD74 (C), CD80 (D), 4-1BB (E), OX40 (F), CXCR3 (G), and VDR (H) among Treg cells from PBMCs and tumors of the same patients (numbers indicate percentages of cells in the indicated gates).

FIG. 12. Expression of Tumor-Specific Treg Targets in CD8+ T Cells, CD4+ T Conventional (Tconv), and Tregs Cells from PBMC and Tumors from NSCLC Patients.
Representative plots of ex-vivo FACS staining show the geometric mean expression (A), or frequency (B), of live CD8+ T cells and CD4+ T conventional (Tconv) and T regulatory cells (Tregs, CD4+ FOXP3+) expressing the indicated markers in matched PBMC and tumors from the same patient. Numbers in (A) indicate the geometric mean expression of CD4, FOXP3 and CD25. Numbers in (B) indicate the percentage of positive cells for CD177, CTLA-4, GITR, TNFR2, VDR, CCR8, 41BB, OX40, CD39, CSF1, CD80, HLA-DR, CXCR3, IL12RB2, CD74, ICOS, and ICAM1. Genes are selected among Table 1 and Table 2.

FIG. 13: OX-40, 41BB, and CCR8 Identify Functional Tumor-Specific Tregs.
Tumor cell suspension of NSCLC patients were stimulated 12h at 37° C. with autologous tumor cells lysate with or without anti-human HLA-DR blocking antibodies, followed by FACS staining. Representative plots showed the frequency of marker expression on the surface of Tregs from tumors of NSCLC patients.

FIG. 14: Representative Dot Plots of CD74 and FoxP3 Expression in Tregs KO for CD74, Gated as FSC-SSC/Singlet/Aqua-/CD3+CD4+/FOXP3+, 12 Days after Nucleofection with Mock (Left Panel) or CD74 (Right Panel) RNA Guides.
Freshly FACS-sorted Tregs (DAPI-CD4+CD25hiCD127lo) obtained from healthy donors PBMCs were expanded 7 days in culture with aCD3/aCD28 beads (ratio 1:1 with cells) and IL-2. Tregs were knock-out for CD74 using the CRISPR/Cas9 approach. Cells were analyzed 12 days after.

FIG. 15: CD74 KO or WT Tregs were Generated as in FIG. 14.
A. Cell counts of expanded WT or CD74 KO Tregs after nucleofection.
B. FACS analysis of expanded WT or CD74 KO Tregs upon 20 days of nucleofection. Representative plots show the frequency of CD74+ and CD74- Tregs (FOXP3+ cells) expressing CD25, ICOS, OX-40, PD1 CD38, HLA-DR, 4-1BB and Ki67.

FIG. 16. Co-Expression of CD74 with MIF Co-Receptors at the Surface of Tregs.
Left panel: representative plot of the gating strategy for CD74+ Foxp3+ Tregs. Right plots: frequency of CXCR4, CXCR2 and CD44 co-expression on CD74+ Tregs.

### EXAMPLES

### Example 1: Identification of Functional Tumor-Specific Treg (Ft-Tregs) Markers

### Material and Methods

### 1. STEP1: Clinical Sample Collection

Matched samples of blood, tumor-draining lymph nodes (TDLNs) and tumors were collected from 5 patients with non-small cell lung cancer (NSCLC) having undergone standard-of-care surgical resection. Samples were characterized by IHC, NGS and detection of genomic abnormalities by Cytoscan. Patients sign a written consent, following European ethical guidance.

### 2. STEP 2: Cell Isolation

Samples were processed within 4 hours after the primary surgery, cut into small fragments, and digested with 0.1 mg/ml Liberase TL (Roche) in the presence of 0.1 mg/ml DNase (Roche) for 30 min before the addition of CO₂ independ medium (GIBCO). Cells were then filtered and mechanically dissociated with a 2.5 mL syringe's plunger on a 40-μm cell strainer (BD) and wash with CO₂ independent medium (GIBCO) 0.4% human BSA.

3. STEP 3: scRNAseq (Transcriptome and TCR)

For each tissue, Tregs (DAPI− CD45+CD4+CD25hi CD127lo) and Tconvs (DAPI− CD45+CD4+CD2510 CD127lo/hi) were FACS-sorted and admixed at a fifty/fifty ratio before loading on a 10× Chromium (10× Genomics). For 2 patients, libraries were prepared using a Single Cell 3′ Reagent Kit (V2 chemistry, 10× Genomics); and for 3 other patients, libraries were prepared using the Single Cell 5′ Reagent kit (Immunoprofiling Kit, 10× Genomics), with an additional step to enrich for V(D)J reads according to the manufacturer's protocol. In both protocols, chips were loaded to recover 10000 cells (5000 Tregs and 5000 Tconvs) per sample.

Single cells were captured into droplets together with gel beads coated with unique barcodes, unique molecular identifiers (UMI), poly(dT) sequences (Single Cell 3′ Reagent Kit) or switch oligo (TSO) sequences (Single Cell 5′ Reagent kit), and all the reagent for the reverse transcription to generate the barcoded cDNA (Single Cell 3′ and 5′ Reagent kit, respectively). The retro transcription occurred in-droplets with the following protocol. cDNA was subsequently recovered from droplets, cleaned up with DynaBeads MyOne Silane Beads (Thermo Fisher Scientific), and amplified with an amplification master mix and enzyme (Single Cell 3′ and 5′ Reagent kit, respectively). Amplified cDNA product was cleaned up using the SPRI select Reagent Kit (Beckman Coulter). cDNA quantification and quality assessment were achieved using a dsDNA High Sensitivity Assay Kit and Bioanalyzer Agilent 2100 System. Then, indexed libraries were constructed following these steps: (1) fragmentation, end repair and A-tailing; (2) size selection with SPRI select beads; (3) adaptor ligation; (4) post-ligation cleanup with SPRI select beads; (5) sample index PCR and final cleanup with SPRI select beads. Library quantification and quality assessment were achieved using a dsDNA High Sensitivity Assay Kit and Bioanalyzer Agilent 2100 System. Indexed libraries were tested for quality, denatured, diluted as recommended for Illumina sequencing platforms and sequenced on an Illumina HiSeq2500 using paired-end 26x98 bp as sequencing mode (Transcriptome or Gene Expression, GEX), targeting at least 50 000 reads per cell.

The single cell TCR amplification and sequencing was performed after 5′ GEX generation using the Single Cell V(D)J kit according to the manufacturer's instructions (10× Genomics). Briefly, V(D)J segments were enriched from amplified cDNA by two human TCR target PCRs, followed by the specific library construction. The TCR enriched cDNA and the library quantification and quality assessment were achieved using a dsDNA High Sensitivity Assay Kit and Bioanalyzer Agilent 2100 System. V(D)J libraries were sequenced on an Illumina Hiseq or Miseq using paired-end 150 bp as sequencing mode.

4. STEP4: scRNA-Seq Transcriptome and TCR Data Analysis
4.1 STEP4.1: scRNA-Seq Transcriptome Analysis by Sample

The paired-end 26x98 bp output from HiSeq Illumina sequencer was processed with cell ranger pipelines for generation of the count matrix and with Seurat v3 for the further analysis.

### Cell Ranger

The pipeline Cellranger mkfastq (default parameters) was run in Cell Ranger version 2.1.1 to demultiplex raw base call (BCL) files from Illumina sequencer and generate FASTQ files.

Sequencing data processing was then performed with Cell Ranger version 3.0.2 pipelines. Cellranger count function was run on each GEM. The reads by GEM were mapped on the human genome (GRCh38/hg38; Genome Reference Consortium Human Build 38 submitted in Dec. 17, 2013; GenBank assembly accession: GCA_000001405.15) using STAR with further MAPQ adjustment, transcriptomic alignment, UMIs counting for each gene, and calling cell barcodes.

The output of Cellranger was then loaded into R.

### Seurat

Seurat 3.1.1 in R 3.6.1 (Butler et al., 2018; Stuart, Butler et al., 2019). After creation of Seurat object from the count matrix, the data followed the pre-processing workflow for selection and filtration of cells based on QC metrics, data normalization and scaling, as well as the detection of highly variable features. After, the samples were individually analyzed following the default parameters of Seurat v3 pipeline.
QC and selecting cells for further analysis

Filter cells with few genes (debris, death cells,): cells with less than 200 genes were removed.

Filter dead cells or doublets trough % of mitochondrial genes and total count UMI/cell: When it was possible, the distribution of the cell counts by 1) the log₂ % mitochondrial genes and 2) the log₂ total count UMI by cell, were fit by a polymodal function. The maximum and minimum values of the function were determined algebraically finding the vertexes or turning points. The % of mitochondrial genes and total count of UMI by cell that corresponded to the lowest minimum value of the function between the two highest maxima, were selected as cutoff. All the cells with higher percentage of mitochondrial genes or total UMI counts per cell than the corresponding cutoff were considered as dead cells or doublets and eliminated. When it was not possible to generate a polymodal function for distribution of % of mitochondrial genes, 10% was used as cutoff.
Normalization

UMI counts per gene of each cell were normalized by the total expression. By default, Seurat uses global-scaling normalization method “LogNormalize” that normalizes the feature expression measurements for each cell by the total expression, multiplies this by a scale factor (10,000 by default), and log-transforms the result.

LogNormalize using NormalizeData function (done by sample) UMI count by cell:gene
Identification of highly variable features (feature selection)

Next, the subset of 2000 features that exhibit high cell-to-cell variation in the dataset was identified using the function FindVariableFeatures. FindVariableFeatures (method: vst, cutoff value for dispersion=0.5; cutoff value for average expression=0) (by sample).
Scaling the data

The data was then linearly transformed (“scaling”) using the ScaleData function that 1) Shifts the expression of each gene, so that the mean expression across cells is 0; 2) Scales the expression of each gene, so that the variance across cells is 1. This step gave equal weight of each gene in downstream analyses, diminishing the impact of highly-expressed genes.
Linear dimensional reduction

To overcome the extensive technical noise in any single feature for scRNA-seq data, Principal Component Analysis (PCA) was performed on scaled data. PCA converts the expression matrix into a set of values of linearly uncorrelated variables called principal components (PC) ordered in function of the variance (from the highest to the lowest). The top principal components therefore represent a robust compression of the dataset. To select the number of significant components, the percentage of variance versus the PCs (ElbowPlot) was visualized and the slope of the linear function between two consecutive values was calculated. The inventors found for each sample the PC for which the aforementioned slope stabilized and after evaluation of all the samples, decided to keep the top 50 PCs.

4.2 STEP4.2: scRNA-Seq Transcriptome Analysis of Integrated Data
Integration

To integrate the different samples (tissues and patients) in their unique dataset that comprised the diversity of Tregs and Tconvs, the inventors used the Seurat v3 integration method. Briefly, this method identifies pairwise correspondences between individual cells (identified as “anchors”) that are used to harmonize pairs of datasets or transfer information from one to another.
Set the Seurat object with the 2000 most variable genes Log normalized as explained above
Identification of anchors in each sample (15 in total: 5 patients×3 tissues) using FindIntegrationAnchors (on the first thirty PCs).
Integration of samples using the Integrate Data that uses the anchors (on the first thirty PCs). The function returned a Seurat object containing new Assay entry as the integrated expression matrix.
Scaling the data (as above)
Linear dimensional reduction (as above)
Cluster the cells

Using Seurat v3, a graph-based clustering approach was applied. Briefly, a KNN graph was constructed based on the euclidean distance in PCA space and the edge weights between any two cells was refined according to the feature overlap in their local neighborhoods (FindNeighbors function in the top 50 PCs). This allows the compartmentalization of the cells in highly connected communities. Then, the modularity of the clusters was optimized, iteratively grouping the cells (Louvain algorithm) with the FindClusters function. This algorithm contains a parameter called “resolution” which determines the “granularity of the clustering” and it is related with the number of clusters obtained. In order to identify the optimal resolution Clustree v.0.2.2 (Zappia, Oshlack, 2018) was performed to visualize the clustering tree allowing the interrogation of the clustering behavior across the different resolutions (graphic representation of the cells movements among clusters as the clustering resolution increased).

FindNeighbors function on the first fifty PCs; FindClusters function to identify the clusters for the resolution between 0 and 2 (for each decimal: 0.1, 0.2, . . . , 2).
Non-linear dimensional reduction (UMAP)

To visualize their high-dimensional data, the inventors used Uniform Manifold Approximation and Projection (UMAP) for two-dimensional visualization, a new algorithm that creates informative clusters and organize these clusters in a meaningful way. McInnes, L. and Healy, J. (2018). UMAP: Uniform Manifold Approximation and Projection for Dimension Reduction.
Global differential analysis

Differentially expressed genes between clusters were identified with FindAllMarkers function using MAST (Finak, McDavid, Yajima et al., 2015) with a minimum Log Fold-Change of 0.25 and a minimum fraction of cells expressing the gene in either of the two groups of cells (min.pct)=0.25 over the integrated matrix.

Parameters for FindAllMarkers: done on integrated data, only.pos=TRUE, min.pct=0.25, logfc.threshold=0.25.
Identification and elimination of contaminants

Cells that did not express T cell markers (CD3E, CD3G, TRAC, TRBC1, and TRBC2) and expressed markers of other populations (CD79A for B cells, CD14 for monocytes, CD11c for Dendritic Cells), were identified as contaminants and removed.
Integration (without contaminants)
The integration approach was reprocessed using the 2000 most variable genes of only CD4+ T cells (ie after removing contaminants) using FindVariableFeatures (method: vst, cutoff value for dispersion=0.5; cutoff value for average expression=0) (by sample). Higher numbers of variable genes were tested for the integration, but 2000 were sufficient to efficiently discriminate the diversity of CD4+ T cell population.
Identification of anchors in each sample (15 in total: 5 patients×3 tissues) using FindIntegrationAnchors (on the first thirty PCs).
Integration of samples using the Integrate Data that uses the anchors (on the first thirty PCs). The function returned a Seurat object containing new Assay entry as the integrated expression matrix.
Clustering the cells (without contaminants)
Graph-based clustering analysis were calculated with first fifty PCs. FindNeighbors function on the first fifty PCs; FindClusters function to identify the clusters for the resolution between 0 and 2 (for each decimal: 0.1, 0.2, . . . ).
Construction of Clustree v.0.2.2 (Zappia, Oshlack, 2018)
Construction of dimensional reduction visualization using RunUMAP function, based PCA reduction on the first fifty PCs.
Adaptation to inventor's data
Resolution: The inventors have chosen the resolution 0.9 because it presented a good compromise between stability and number of clusters with biological interest.
Overview of Clusters: The inventors have checked the percentage of cells per sample and/or patient in order to identify and remove for further analysis the clusters that were exclusive of one sample or patient. The inventors found that the cluster 19 contained 98% of cells coming from only one sample (Tumor 18P05408) and the inventors did not consider it for further analysis.
Non-linear dimensional reduction (UMAP)
Characterizing the clusters
Global differential analysis: Differentially expressed genes among clusters were identified with FindAllMarkers function using MAST (Finak, McDavid, Yajima et al., 2015) with a minimum Log Fold-Change (FC) of 0.25 and a minimum fraction of cells expressing the gene in either of the two groups of cells (min.pct)=0.25.
Parameters for FindAllMarkers function: Object: integrated data, only.pos=TRUE, min.pct=0.25, logfc.threshold=0.25.
Exploration of cell identity
Cellular identity of each cluster was determined using marker genes expression and enrichment of relevant signatures. Signature scores were calculated for each relevant signature with AddModuleScore function using the number of genes/2 as ctrl parameter.
The inventors corroborated that the clusters 2 and 18 displayed high overlap in the expression profile with really few differentially expressed genes, and were unified. To note, they were fused in the resolution 0.8.
Clusters were classified as Tregs, Tconvs and Tmix using Treg signatures and key genes (FOXP3, IL2RA, CTLA4, and TNFRSF1B for Tregs; IL7R and CD40L for Tconvs). When cells in the cluster showed enriched scores for signatures and genes of Tregs and low scores for genes and signatures of Tconvs, this was classified as “pure” Treg (1-5). Similarly, for “pure” Tconv (1-7). The clusters with mixed characteristics were called Tmix. The Tmix annotation was corroborated using transfer label function with: two big clusters as reference: all Tregs as a single cluster (Tregs clusters: 1-5) and all Tconvs clusters as a single cluster (Tconv 1-7); and the individual mixed clusters as query.
   4.3 STEP4.3: scTCR analysis

Single cell 5′ gene expression and V(D)J sequencing was demultiplexed and aligned with Cell Ranger v.2.1.1 using GRCh38/hg38 as reference with the function cellranger mkfastq. Cellranger vdj function was then run in Cell Ranger v.3.0.2 and used to perform V(D)J sequence assembly. The inventors obtained as output: the TCR alpha (TRA) and beta (TRB) V(D)J sequences, the cell barcode and the CDR3 sequence (nucleotides).

To generate the clonotype calling, the inventors created a CDR3 nucleotide sequence database that considers separately the TRA and TRB chains. The inventor's database contains different identifiers for each clonotype or collection of cells that share a set of productive CDR3 sequences by exact match: the TRB identifiers (IDs) based on the TRB-CDR3 unique sequences, and the TRA sub-identifiers (sub-IDs) based on the TRA-CDR3 unique sequences.

The inventors used their database to improve the calling of the clonotypes and better identify cells that belong to the same clonotype, overcoming the common TRA dropout and considering the absence of allelic exclusion in TRA rearrangement. By patient:
Cells with more than 2 sub-IDs (TRA-CDR3 sequences) and/or more than 1 ID (TRB-CDR3 sequences) were excluded as probable doublets.
Cells containing the same ID (TRB-CDR3 sequences) were considered as a clonotype if in whole the cells sharing the same ID presented at maximum 2 sub-IDs (TRA-CDR3 sequences).
Clonotypes containing 1 or 2 sub-IDs (TRA-CDR3 sequences) and 0 ID (TRB-CDR3 sequences), were searched in the database.
If they were not found, they were not included for TCR analysis.
If they were found, they were evaluated in relation to the pairing with the TRBs-CDR3:
   If they were unique, we assigned the paired ID found in the database.
   If they were not unique, they were not included for TCR analysis.

Common clonotypes between tumor, lymph node and PBMC samples from the same patient were also identified with our strategy. Pairing transcriptomic and V(D)J information was made sample by sample using the cell barcodes.

### 4.4 STEP4.4: Clonal TCR Expansion Analysis

With the TCR information by cell, the inventors first interrogated the clonal expansion by tissue. The inventors identified the list of unique clones by tissue and counted the number of cells by clonotype in this tissue. When clones contained more than one cell, they were considered as expanded. The percentage of expanded clones by tissue for each patient was calculated as:

% of expanded clones by tissue=#of expanded clones/Total clones

With the paired cluster (obtained from scRNA-seq transcriptome analysis) and TCR information, the inventors then calculated the percentage of tumor-expanded clones by cluster. With the list of the unique tumor-expanded clonotypes obtained before, the inventors selected the cells present in all the 3 tissues and classified them according to their cluster label.

The percentage of cells with tumor-expanded clonotypes by cluster (for each patient) was calculated as:

% of cells with a tumor-expanded clonotype in cluster N=#of cells with a tumor-expanded clonotype in the cluster N/#total cells in the cluster N;

### 5. STEP5: Identification of Functional Tumor-Specific Tregs

Functional tumor-specific Tregs (FT-Tregs) were defined as cells that belong to a cluster (or group of cells) with all the following characteristics:

5.1 A cluster of cells bearing characteristics of CD4+ FOXP3+ Tregs, and

5.2 A cluster of CD4+ FOXP3+ Tregs that are found in the tumor or in the tumor-draining LNs (in particular metastatic tumor-draining LNs) at higher proportions than in the blood (i.e. that accumulates in tumor or in TDLN), and

5.3 A cluster of CD4+ FOXP3+ Tregs that is enriched in cells with specificities (TCRs) that are found clonally expanded in the Treg cells from the tumor, and

5.4 A cluster of CD4+ FOX3P+ Tregs that is enriched in cells with a transcriptomic signature of recent TCR triggering, cell activation and expansion in the Treg cells from the tumor.

Thus, this method helps to classify Tregs in functional subsets and distinguish functional tumor-Treg clusters out of the heterogeneous pool of Tregs.

### 6. STEP6: Identification of Specific Markers of Tumor-Specific Tregs

Tumor-specific Tregs were defined as cells with tumor-expanded clonotypes present in the Treg cluster4, and their transcriptome was identified by analysis of unique differentially expressed genes (DEG) in this population. First, zero counts and heterogeneity of the data were dealt with the statistical tool MAST (Finak, McDavid, Yajima et al., 2015). Second, to cope with fact that analysis of DEGs between clusters composed of few cells and big clusters biases the data towards the biggest clusters, the inventors designed a strategy in two steps. First, the inventors defined the DEGs between the tumor-specific Tregs (as defined above: cells with tumor-expanded clonotypes present in the Treg cluster 4 from all patients) and each of the other clusters independently. Second, the inventors added up all the DEGs (intersection of all comparisons). The differentially expressed gene analysis between groups of cells was performed using the original data (not integrated) with FindMarkers function using MAST, with Bonferroni p value correction inferior or equal to 0.05, a minimum Log Fold-Change of 0.2, and min.pct=0.05.

### 7. STEP7: Identification and Ranking of Tumor-Specific Treg Markers for Therapeutic Purpose

For the selection of tumor-specific Treg markers, the inventors defined a larger list of DEGs between the tumor-specific Tregs (as defined above: cells with tumor-expanded clonotypes present in the Treg cluster 4 from all patients) and all other clusters (all Tconvs clusters and all Treg clusters except Treg4), using FindMarkers function with MAST, and Bonferroni p value correction inferior or equal to 0.05, a minimum Log Fold-Change of 0.12, and min.pct=0.05.

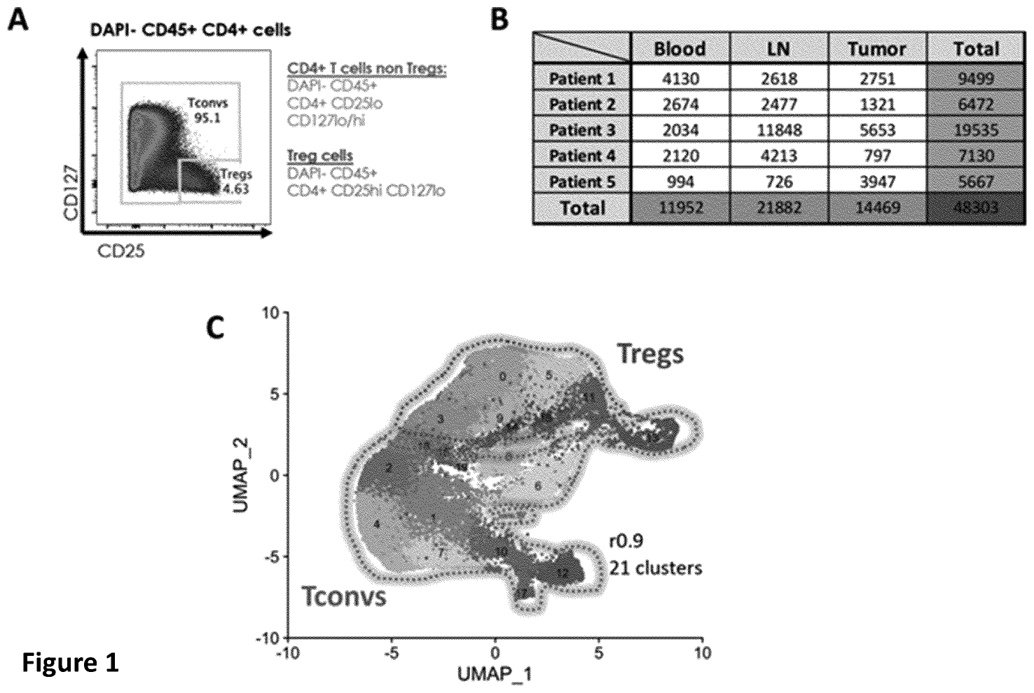

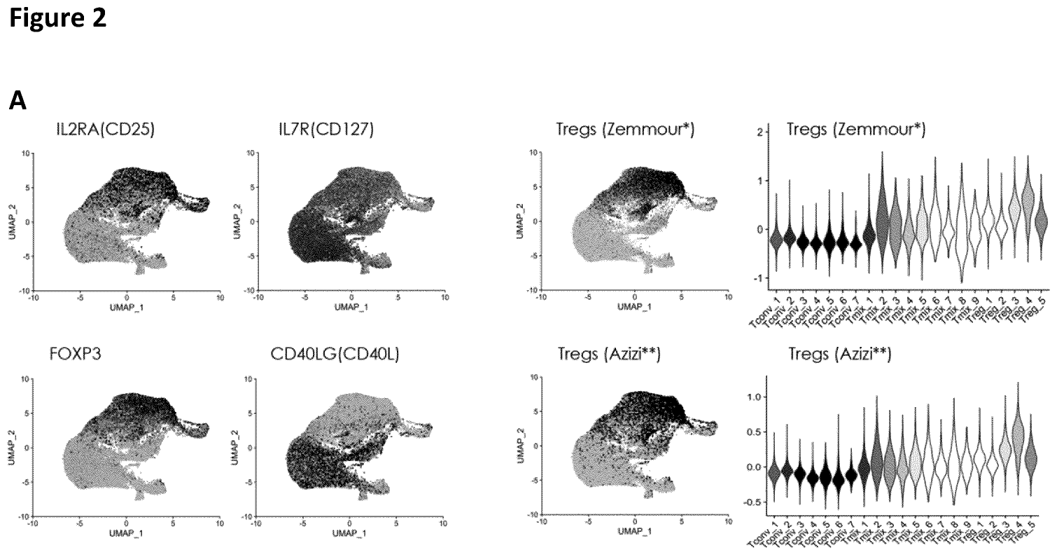

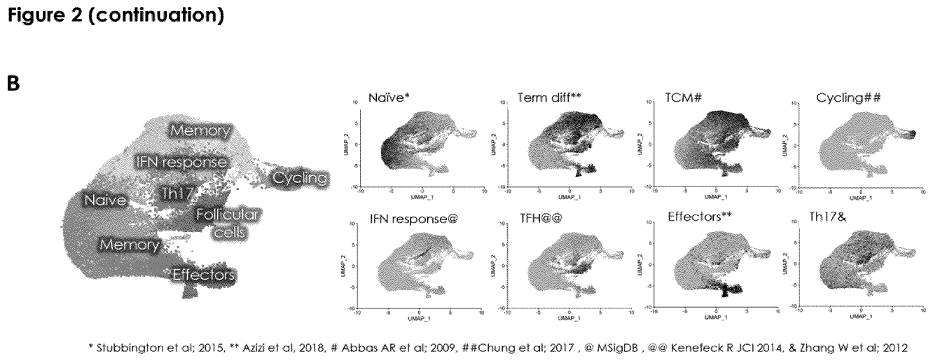

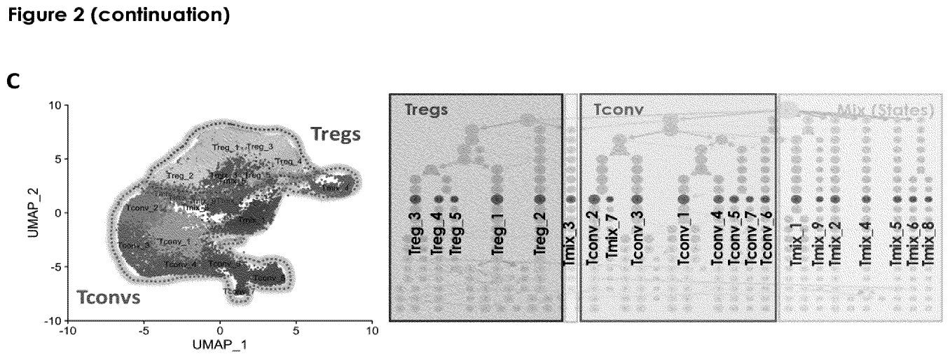

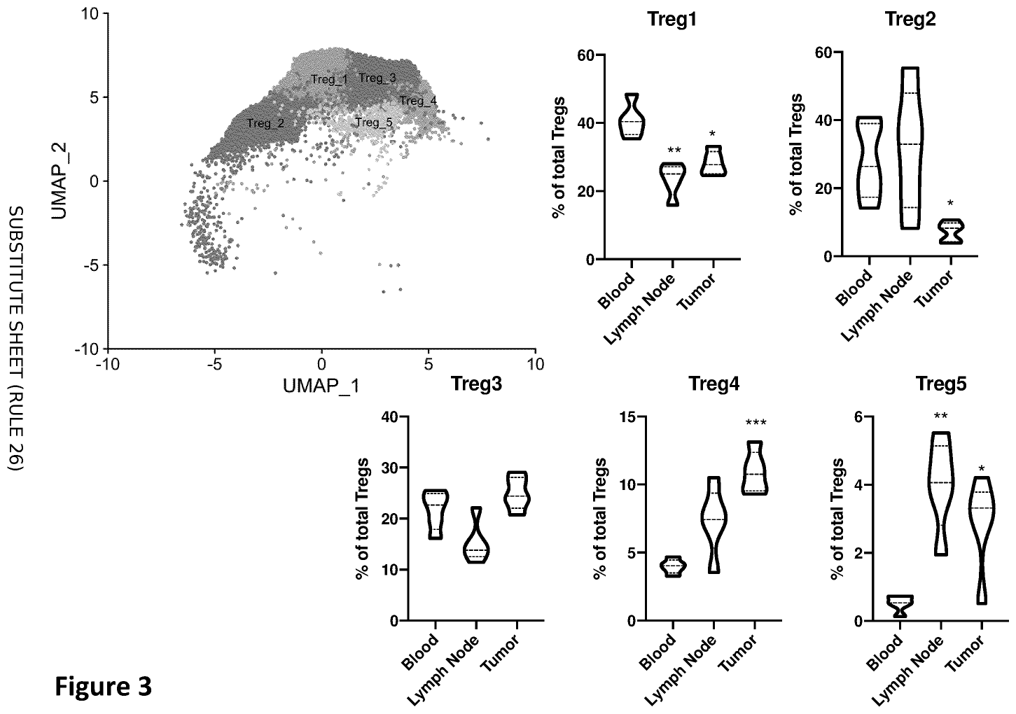

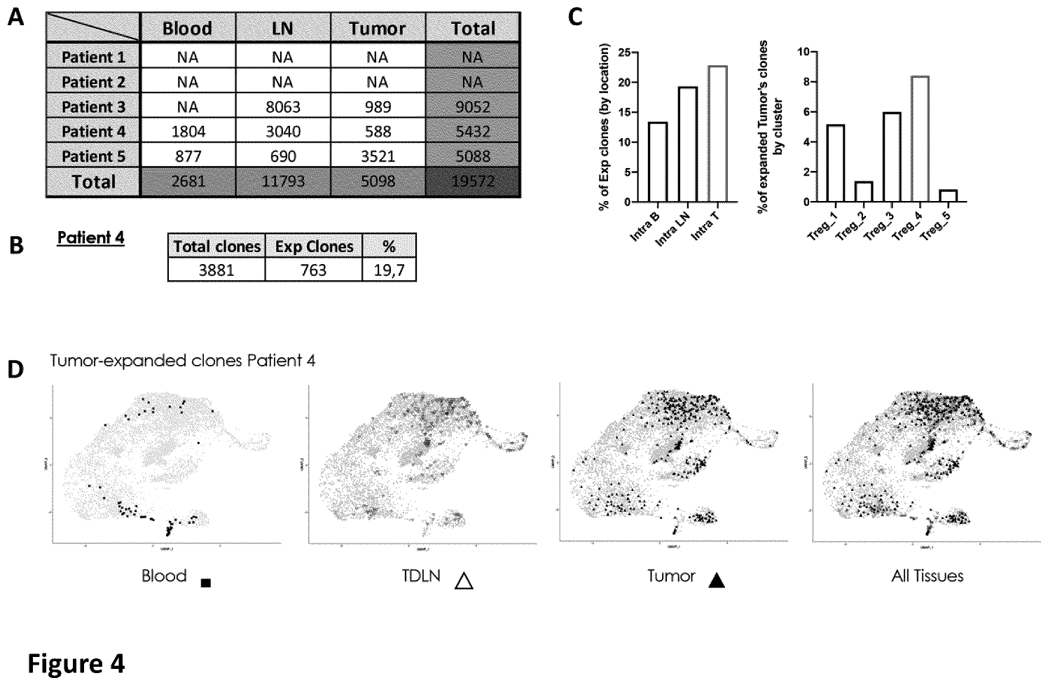

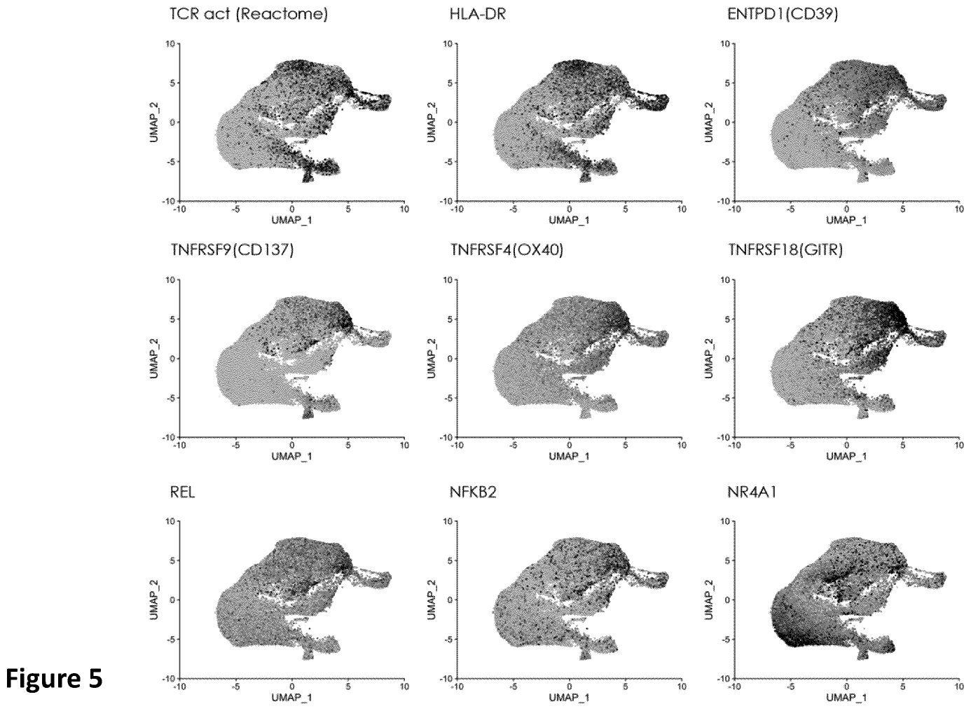

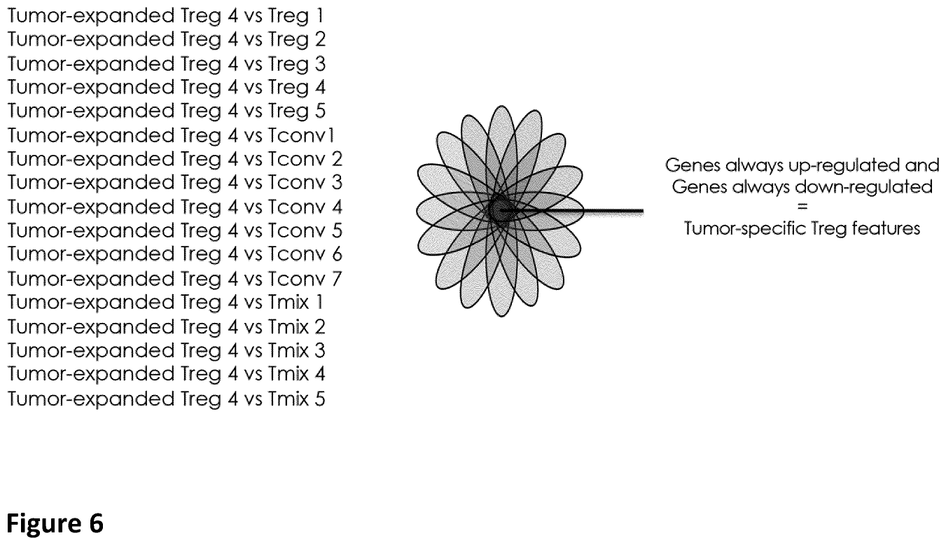

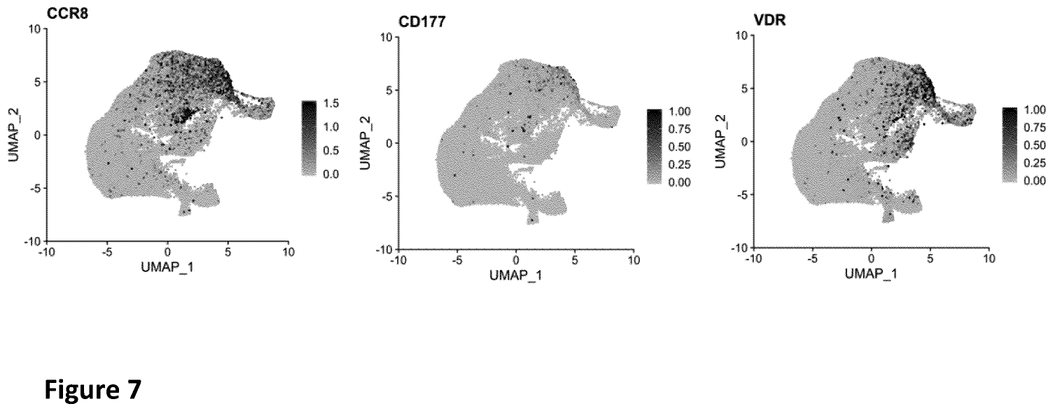

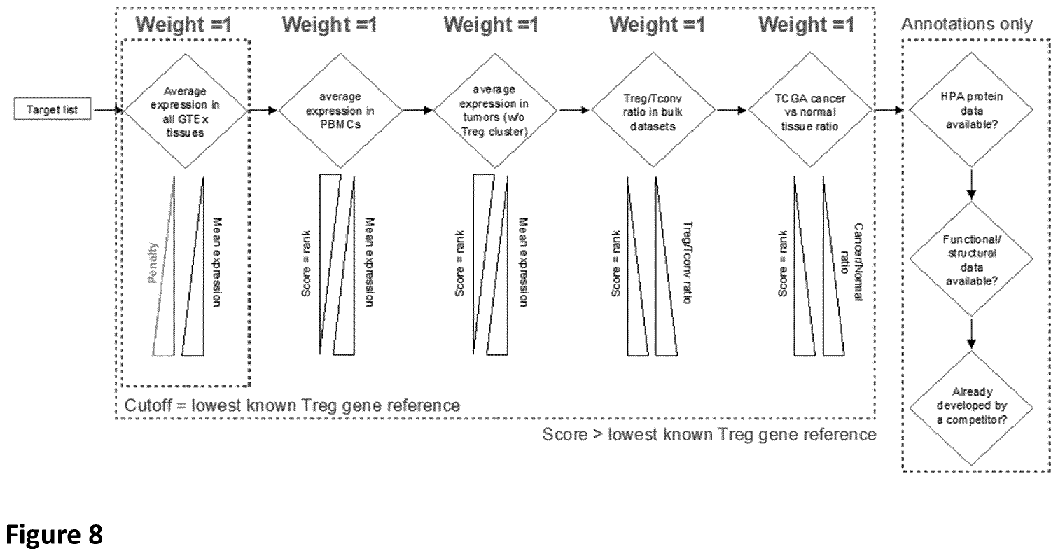

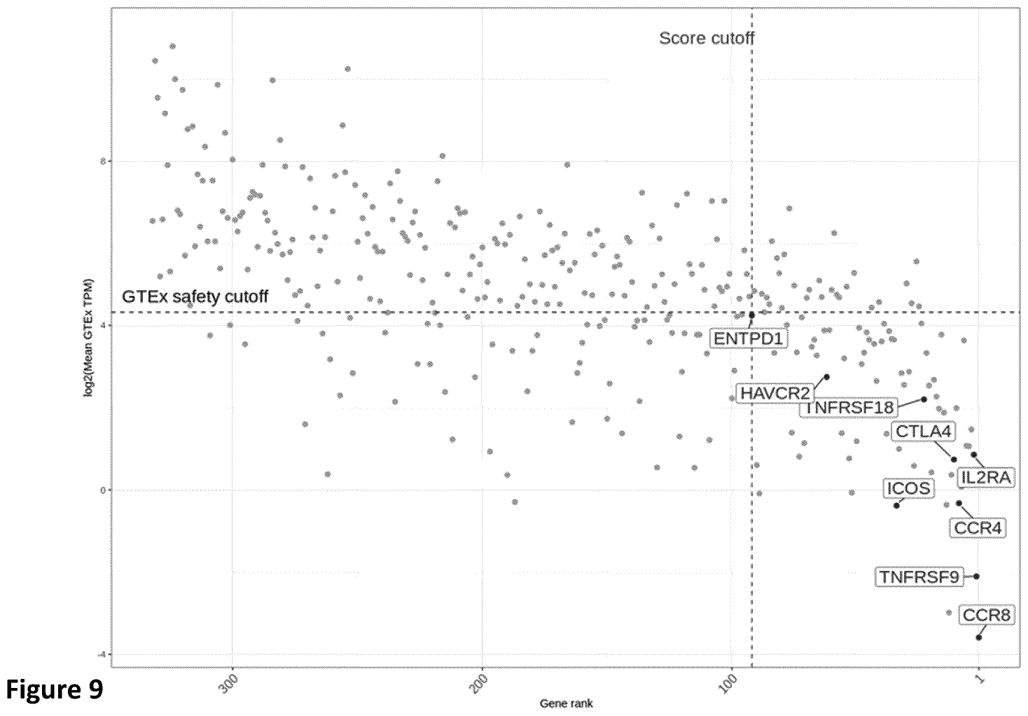

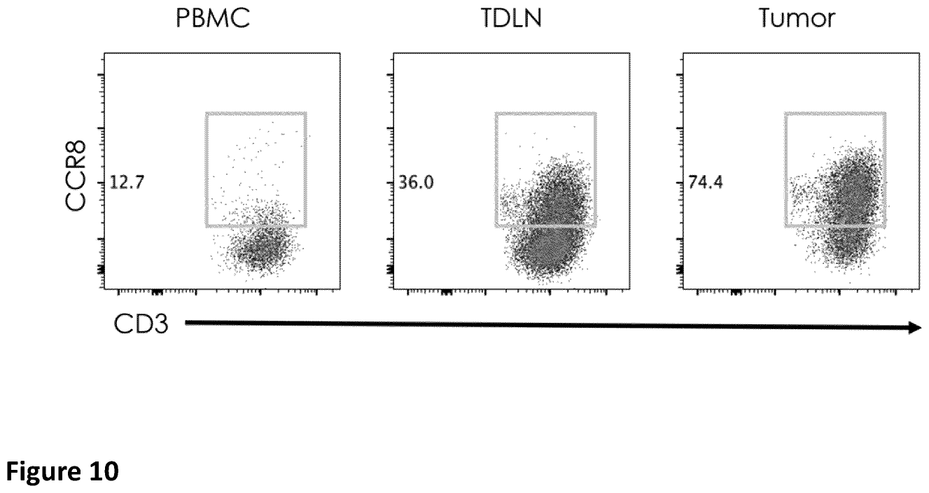

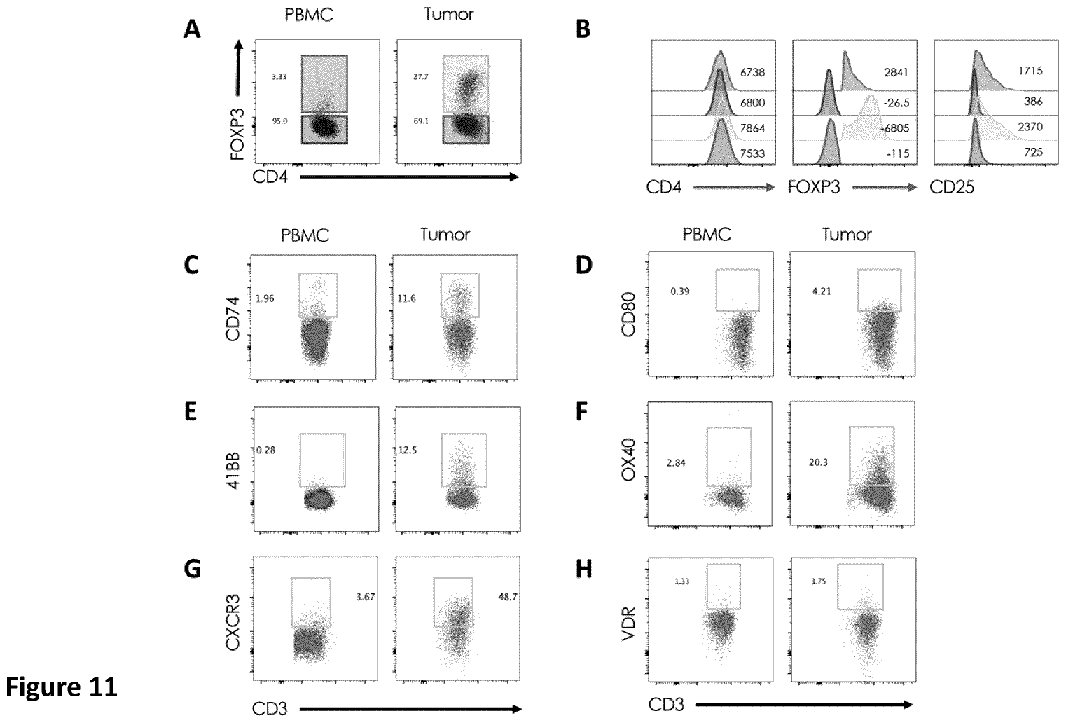

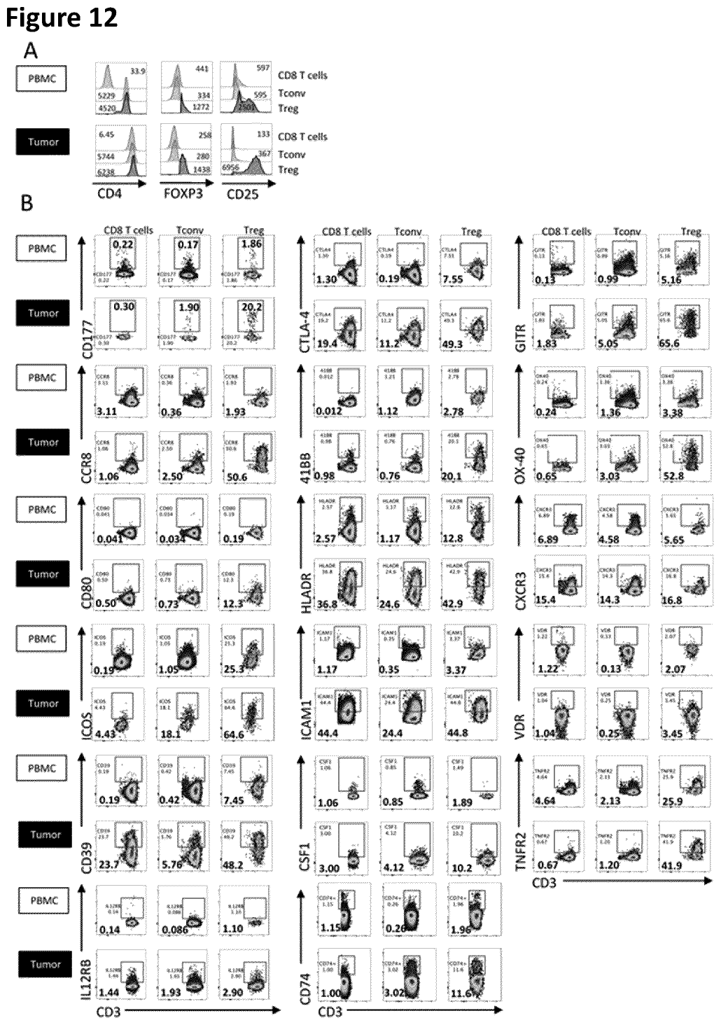

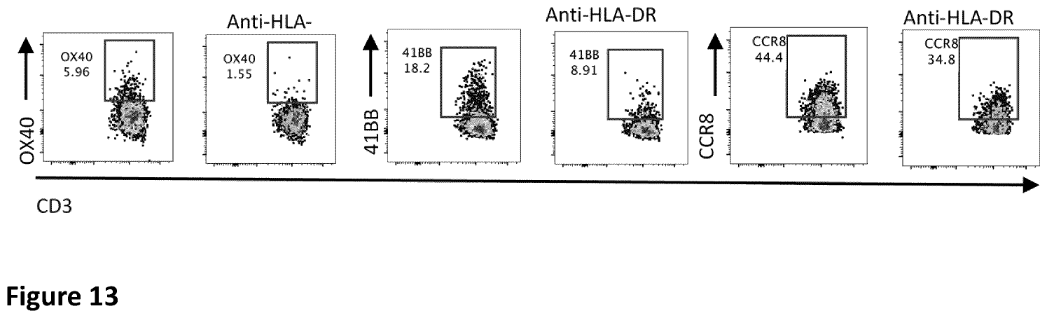

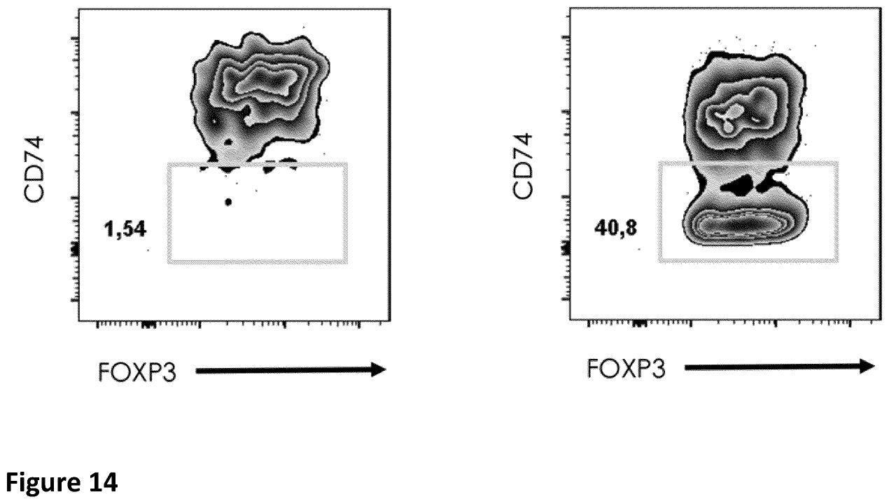

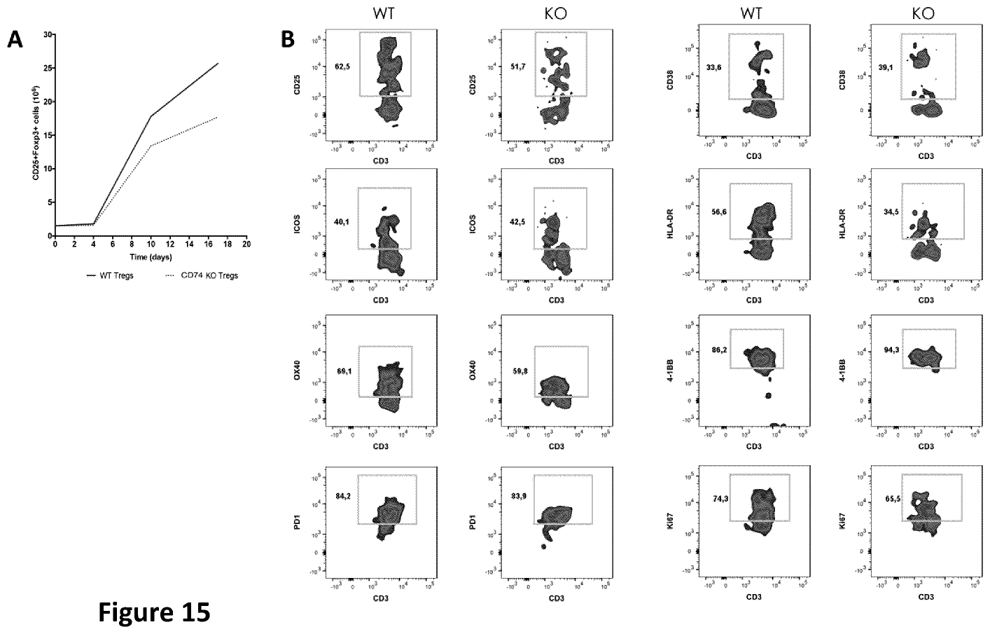

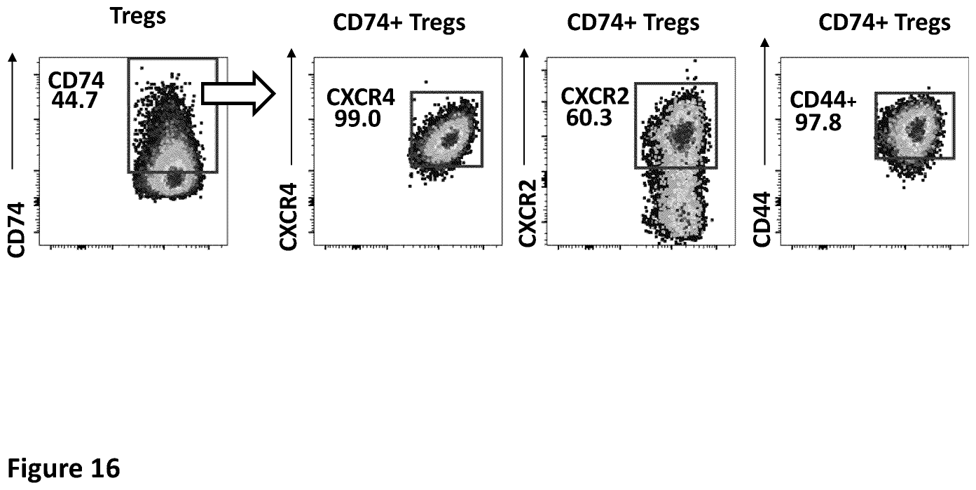

This new list includes all genes of STEP6 and other genes, that are then prioritized using a novel bioinformatics pipeline consisting of 6 stages as illustrated in FIG. 8:

BioIT Stage 1: Filtering of the initial list of all differentially expressed genes, to extract only those coding for transmembrane or GPI-anchored proteins with a confirmed extracellular domain.

For that, an annotation table was created with information extracted for 3 sources, and using the following commands:
Source: Uniprot: Subcellular localization contains “Cell membrane”: TRUE/FALSE
Subcellular localization contains “GPI-anchor”: TRUE/FALSE
Topological domain contains “Extracell”: TRUE/FALSE
Transmembrane contains “TRANSMEM”: TRUE/FALSE
Source: Gene Ontology
Cellular component contains “plasma membrane”: TRUE/FALSE
Source: Human protein atlas:
Protein class contains “membrane”: TRUE/FALSE
Protein main localization contains “Plasma membrane”: TRUE/FALSE
Protein additional localization contains “Plasma membrane”: TRUE/FALSE

All genes with at least one positive keyword were investigated using Protter (https://wlab.ethz.ch/protter/), a web tool allowing the visualization of a given protein amino-acid wise and its membrane localization. Using this approach, n=333 genes (which correspond to around 10% of all the genes differentially expressed) were confirmed as coding for potential transmembrane proteins with a confirmed extracellular domain.

BioIT Stage 2: Weighing the Target Expression in Normal Tissue

First the profile of expression of each target was determined at the tissue level in healthy tissues. The Genome Tissue Expression (GTEx) database (V8 release, TPM) was used to calculate a score of expression in healthy tissue for each target. All tissues from GTEx (with the exception of immune related tissues “whole blood” and “spleen” for which we have better resolution using single cell data) were first averaged for over each tissue type to avoid bias from tissues that have several entries (corresponding to sub-localization within the tissue). The average expression of each target was then calculated along all summarized tissue. A score of penalty was attributed to each of the 333 targets (1 for the best, 333 for the worst), to account for their expression in healthy tissues.

BioIT Stage 3: Weighing the Target Expression in Tumoral Tissue

Each target expression was analyzed in diseased tissues using The Cancer Genome Atlas (TCGA) RNAseq data. Given that for several tissue types the number of healthy samples to compare the cancer samples to was insufficient, TCGA data was supplemented with data from healthy samples extracted from the GTEx database. To correct the batch effect inherent to the comparison of the two databases, a normalization method has been developed consisting in using normalized counts of the recount2 resource from TCGAbiolinks (Mounir et al., PLoS Comput. Biol., 2019, 15, e1006701), corrected for library size, RNA composition and gene length using edgeR (McCarthy et al., Nucleic Acids Res., 2012, 10, 4288-4297) and then corrected again for batch effect using Limma (Ritchie et al., Nucleic Acids Res., 2015, 43, e47). Correct alignment of the two databases has been verified in several tissues by principal component analysis. For each target, the fold change of the median of cancer samples (from TCGA) vs the median of healthy samples (including both TCGA & GTEx samples) was calculated in 3 main cancer types: Lung, Breast and Colon. Each target was given a score depending of their rank for the average fold change Cancer/Healthy in the previously mentioned cancers, (333 for the best, 1 for the worst).

BioIT Stage 4: Weighing the Target Expression in Data Obtained from Single-Cell RNA Sequencing of Healthy Donor PBMCs

The initial differential analysis led to the identification of genes that are differentially expressed by functional tumor Tregs, but gave no information on the expression of these genes by other immune cells. Hence, a workflow has been developed to identify genes that are not only differentially expressed by functional tumor Tregs but also expressed at very low level in all PBMCs. For this, the expression pattern of each target at the single cell level of peripheral blood mononuclear cells (PBMCs) was analyzed using two publicly available different datasets of PBMCs profiled using 10× genomics and comprising 5,000 and 10,000 cells, respectively.

First, the PBMCs datasets were analyzed to a depth that allowed the identification of the Treg cluster in the blood. All cells from this cluster were then removed from the datasets. On the remaining cells, the average expression of each target was calculated on each cluster individually and then the mean of cluster averages was calculated for each target in each dataset. This intermediate step avoids any cluster size bias in the analysis. Each target was given a score dependent of its rank for the average expression in all PBMCs (except Tregs that were removed) in both datasets, (333 for least expressed, 1 for most expressed).

BioIT Stage 5: Weighing the Target Expression in Data Obtained from Single-Cell RNA Sequencing of Cells from the Tumor Microenvironment

To characterize the expression pattern of each target in the tumor microenvironment at the single cell level, publicly available single-cell RNAseq was obtained using 8 datasets from 7 publications (Azizi et al., Cell, 2018, 174, 1293-1308; Li et al., Cell, 2019, 176, 775-789; Yost et al., Nat. Med., 2019, 8, 1251-1259; Guo et al., Nat. Med., 2018, 24, 978-985; Zheng et al., Cell., 2017, 169, 1342-1356; Sade-Feldman et al., Cell., 2018, 175, 998-1013; Peng et al., Cell. Res., 2019, 9, 725-738) covering a wide range of tumor types (NSCLC, Breast cancer, PDAC, Melanoma, HCC, SCC, BCC . . . ) and also a wide range of cell types (all immune cells but also tumor cells, epithelial, endothelial, cancer-associated fibroblasts and tissue-specific cell types). A similar approach to the one used for PBMCs (STEP4) was adopted. Since the aim of this stage was to identify Treg-specific targets, each dataset has been processed up to a resolution where a Treg cluster could be identified. Tregs were then removed from the datasets and the average expression of each target among all cells (without Tregs) was calculated. Each target was given a score depending of its rank for the average expression in all cells of the tumor microenvironment (except Tregs that were removed) in all 8 datasets, (333 for least expressed, 1 for most expressed).

BioIT Stage 6: Weighing the Target Expression in Tumor Vs Normal Adjacent Tissue

To measure i) the ability of each target to distinguish between Tregs and Tconv, and ii) evaluate the distribution of the target among Tumor-Tregs and Normal tissue-Tregs, bulk RNAseq data from sorted cell population was analyzed. For that, publicly available bulk RNAseq data was recovered from 2 studies on Breast, Lung and Colon cancer (Plitas et al., Immunity, 2016, 45, 1122-1134; De Simone et al., Immunity, 2016, 45, 1135-1147). For each dataset, each target was given 2 scores. The first one reflecting its rank when calculating the fold change of Treg/Tconv expression, and the second one reflecting its rank when calculating the fold change of Tumor Treg/Normal adjacent tissue Treg expression, (333 for highest fold change, 1 for lowest).

BioIT Stage 7: Data Integration

Upon all these analyses, each target was characterized as followed:
1 penalty score for GTEx expression
1 score for TCGA expression
2 scores for normal single cell RNAseq PBMCs expression
8 scores for single cell RNAseq cancer expression
4 scores for bulk RNAseq cancer expression

As all analyses need to be equally weighed, all scores were averaged (mean) to define only one value for each parameter.

Genes were then ranked by their overall score:

Score=Σ(TCGAscore, scPBMCscore, scTUMORscore, bulkTUMORscore)−GTEXpenalty

Each target was then characterized in term of safety (GTEx average score) and interest (SUM score of all parameters). To define cutoffs of both, a list of described activated-Treg targets were used (IL2RA, ICOS, TNFRSF18, CCR8, CCR4, CTLA4, HAVCR2, ENTPD1, TNFRSF9). Cutoffs for both safety and interest were set as the value of the lowest ranked reference genes.

Following the whole process described above, n=83 targets were defined as “of potential interest”.

BioIT Stage 8: Associated Annotation for Each Target

To complete the profile of the potential of each gene for therapeutic targeting, information in terms of structure, function, availability of reagents, and competitive landscape is manually curated (data mining) and presented in a standardized file

### Results

### STEP 5.1— Identification of Clusters of Cells Bearing Characteristics of CD4+ FOXP3+ Tregs

The inventors focused on non-small cell lung cancer (NSCLC), as it remains one of the most frequent cancers in adults, it is currently treated with immunotherapies, Tregs are associated with poor clinical outcome. The inventors setup the 10×-genomics sc-RNAseq with TCR coupled to transcriptome (@Chromium 10× Immunoprofiling kit) and the bioinformatics pipeline for its analysis using the new method disclosed above.

The result of the analysis performed on CD4+ T cells sorted from 15 samples (blood, TDLN and tumor), obtained from 5 untreated NSCLC patients (48303 single cells) is shown in FIG. 1.

CD4+ T cony cells were identified as expressing CD40L, and CD127, and Tregs were identified as expressing FOXP3, CD25, and expressing genes of published Treg signature (* Zemmour et al., 2018 and ** Azizi et al, 2018; FIG. 2A). CD4+ T cells showing a naïve phenotype were identified using the published signature in Stubbington et al., 2015; terminally differentiated cells were identified using the published signature in Azizi et al, 2018; central memory cells were identified as in Abbas A R et al., 2009, cycling cells as in Chung et al., 2017, cells with an IFN-response signature were identified as in MSigDB, T follicular helper cells as in Kenefeck R, JCI, 2014, and Th17 cells as in Zhang W et al., 2012 (FIG. 2B). The final cluster classification of T cells shows that a total of 7 pure Tconv cell clusters were identified (Tconv clusters 1-7), a total of 5 pure Treg clusters were identified (Treg clusters 1-5) and a total of 9 «mixed T cell» clusters were identified, which were composed of mixtures of cells with Treg and Tconv characteristics (Tmix 1-9; FIG. 2C).

For the rest of the analysis, and with the aim of identifying the clusters containing the tumor-specific Tregs, only pure Treg clusters; i.e. Treg clusters 1, 2, 3, 4 and 5 are considered, because clusters containing mixed Treg and Tconv populations are not informative for the selection of tumor-specific Tregs.

STEP 5.2— A Cluster of CD4+ FOXP3+ Tregs that are Found in the Tumor or in the Metastatic Tumor-Draining LNs at Higher Proportions than in the Blood (i.e. that Accumulates in Tumor or in TDLN)

The inventors hypothesized that tumor-specific Tregs should be present in increased proportions in the tumor tissue or in TDLNs, compared to the blood (where tumor-specific Tregs will be diluted among Tregs with other specificities). To identify which Treg clusters were found in increased proportion in the tumor, the inventors compared the percentages of total Tregs of each pure Treg cluster among the 3 tissues. As observed in FIG. 3, only the proportions of clusters 4 and 5 were statistically significantly increased in tumors, and cluster 5 also in TDLNs, compared with the blood (paired-t test <0.05), suggesting that tumor-specific Tregs should be enriched in clusters 4 and/or 5.

STEP 5.3— A Cluster of CD4+ FOXP3+ Tregs that is Enriched in Cells with Specificities (TCRs) that are Found Clonally Expanded in the Treg Cells from the Tumor

The inventors hypothesized that tumor-specific Tregs should be clonally expanded, as upon recognition of the tumor antigens via their TCR, they should be activated, divide, and locally accumulate. To explore the clonal diversity of Tregs, the inventors studied their TCR repertoire. TCR repertoire analysis was successfully performed in 19572 cells. Results of the integration of transcriptomic and TCR data for each single cell is shown FIG. 4A.

As exemplified for one patient (FIG. 4-D), the 5432 detected cells with paired TCR (containing both alpha and beta TCR chains) and transcriptome presented 3881 different TCRs (clones). 19.7% of all clones (763) were expanded (one clone is considered to be expanded when 2 or more cells with the same TCR are detected). In the tumor, more than 20% of T cell clones were expanded (non-shown).

To identify which Treg clusters are enriched in specificities that are clonally expanded in the tumor, the inventors analyzed the proportion of cell bearing tumor-expanded TCRs within each Treg cluster. As depicted in FIG. 4C, among the 5 pure Treg clusters, clusters 1, 3 and 4 showed higher proportions of T cells bearing tumor-expanded TCRs, being cluster 4 the most enriched in tumor-TCR specificities, as can be appreciated by the UMAP projection of cells bearing tumor-TCR expanded clones (FIG. 4D). Similar results were obtained for the other patients (not shown).

In the step 5.2, the inventors defined that tumor-specific Tregs should be enriched in clusters 4 and/or 5. Given that Treg cluster 4 (but not Treg cluster 5) is enriched in tumor-TCR expanded clonotypes, the inventors conclude that Treg cluster 4 is enriched in tumor-specific Tregs.

The inventors also observed that T cells of the same clone were present in the different tissues at the same time (confirming T cell circulation among blood, TDLN and tumor) and that some Tconvs and Tregs share the same TCR, allowing the study of Treg conversion in humans.

STEP 5.4— A Cluster of CD4+ FOX3P+ Tregs Enriched in Cells with Transcriptomic Signature of Recent TCR Triggering, Cell Activation and Expansion in the Treg Cells from the Tumor.

In this method, tumor-specific Tregs should be clonally expanded, as upon recognition of the tumor antigens via their TCR, they should be activated, divide, and locally accumulate. Consequently, their transcriptome should reflect these biological pathways. For example, recognition of cognate antigens via their TCR should induce among others, the upregulation of genes downstream TCR activation such as REL, NKKB2, NR4A1, OX-40, 4-1BB, and known genes of Treg activation such as MHC class II molecules (HLA-DR), CD39, CD137, GITR. As observed in FIG. 5, these features are enriched in the Treg cluster 4 (as visualized in the UMAP projection). Also, these genes are differentially upregulated in this cluster (see results below), pointing out Treg cluster 4 as the “tumor-specific Treg cluster”.

### STEP6: Identification of Specific Markers of Tumor-Specific Tregs

Tumor-specific Tregs were defined as cells with tumor-expanded clonotypes present in the Treg cluster4, and their transcriptome was identified by analysis of unique differentially expressed genes (DEG) in this population as described in material and methods section above.

As illustrated in the FIG. 6, the DEG analysis was done comparing the cells with tumor-expanded clonotypes present in the Treg cluster 4 (from all the patients together) versus the cells belonging to individual clusters (Treg 1-5; Tconv 1-7, Tmix 1-7). From the intersection of all these 19 DEGs, the inventors only kept the genes that changed always in the same direction (always up-regulated or always down-regulated). The genes always up-regulated or always down-regulated were considered as the tumor-specific Treg features. An exemplary and non-exhaustive list of Tumor-specific genes is included in Table 1.

FIG. 7 shows the UMAP projection of some selected genes from the list in Table 1. As referred above, the differentially expressed genes (DEGs) upregulated specifically in the “Treg cluster 4 expanded” included TCR activation genes and Treg activation markers, and some of the genes in this list have not previously been associated to Treg biology.

### STEP7: Identification and Ranking of Tumor-Specific Treg Markers for Therapeutic Purpose

For the selection of tumor-specific Treg markers, the inventors defined a larger list of DEGs between the tumor-specific Tregs (as defined above: cells with tumor-expanded clonotypes present in the Treg cluster 4 from all patients) and all other clusters (all Tconvs clusters and all Treg clusters except Treg4), using FindMarkers function with MAST, and Bonferroni p value correction inferior or equal to 0.05, a minimum Log Fold-Change of 0.12, and min.pct=0.05.

Following the whole process described above, n=83 targets were defined as “of potential interest” (FIG. 9).

### Example 2: Validation of Tumor-Specific Treg Markers

### 1. Validation of the Protein Expression Level of Tumor-Specific Treg Markers

To validate the methodological approach, the protein expression level of candidate tumor-specific genes was evaluated by FACS, comparing the level of expression in Tregs from blood vs Tregs from TDLN and the tumor. As exemplified in FIG. 10, we the protein expression level of CCR8 (as model candidate tumor-specific Treg gene present in the Treg 4 cluster) was analyzed on Tregs from blood, TDLN and tumor of one NSCLC patient. It can be observed that the percentages of Treg cells positive for this candidate protein increased from blood, to TDLN and Tumor, as predicted by the scRNAseq results.

As exemplified in FIG. 11, the inventors have analyzed the protein expression level of other candidate tumor-specific markers from the list in Table 1, namely: CD4, FOXP3, CD25, CD74, CD80, 4-1BB (TNFRSF9), OX40 (TNFRSF4), CXCR3 for some of which little information exist on their role in Treg biology (Cantorna et al., Nutrients, 2015, 7, 3011-3021; Chambers and Hawrylowicz, Curr. Allergy Asthma Rep., 2011, 11, 29-36; Xu et al., Front. Immunol. 2018, 9). As predicted by their method, all the exemplified genes are more expressed in tumor Tregs than in blood Tregs, what can be observed at the expression level (mean fluorescence intensity, MFI, FIG. 11A-B) and/or at the percentage of cells expressing the marker (% of cells in FIG. 11C-G). These results highlight the validity of our method. Furthermore, as exemplified in FIG. 12 the inventors have analyzed the protein expression level of some candidate tumor-specific markers from the lists in Table 1 and Table 2 in CD8+ T cells, CD4+ T conventional (Tconv), and Tregs cells from PBMC and tumors from NSCLC patients. As predicted by their method, all the exemplified genes are more expressed in tumor Tregs than in blood Tregs, and that in CD8+ T cells from blood and tumor, what can be observed at the expression level (mean fluorescence intensity, MFI, FIG. 12A) and/or at the percentage of cells expressing the marker (% of cells in FIG. 12B). These results highlight the validity of our method.

2. Validation that the Identified Tumor-Associated Treg Markers are Associated to Tumor-Specific Tregs.

One approach to evaluate the specificity of human Tregs is to co-culture them with a lysate of autologous tumor cells and analyze the expression of induced molecules and control that their expression is not induced in the presence of blocking antibodies to HLA-cII molecules. As exemplified in FIG. 13, the inventors have analyzed the expression of selected markers form the list in cells that are specifically recognizing autologous tumor antigens, and they could observed that OX-40, 41BB, and CCR8 effectively marks tumor-specific Tregs.

### 3. Evaluation of the Role of the Target Proteins in the Biology of Human Tregs

One approach to evaluate the role of the target markers in the biology of human Tregs, is to Knock-out the candidate gene in primary human Tregs, for example by using the CRISP/CAS9 technology. As an example, the inventors used CRISPR (clustered, regularly interspaced, short palindromic repeats)/Cas9 (CRISPR-associated protein) to knock out in primary human Tregs one example candidate gene selected from their list: CD74. For this, Tregs (CD4⁺CD127⁻CD25^{high}) were FACS-sorted from healthy-donor PBMCs and expanded in vitro during 2 days with CD3/CD28 beads and IL-2. Then, 2×10⁶ Tregs were transfected with chemically modified synthetic target gene-specific CRISPR RNAs (crRNA) using one guide RNA and tracer RNA, the latter mediating the interaction with Cas9. Cells treated without dsRNA (Mock) were used as a negative control (WT). Efficacy of knock out was evaluated by measuring the percentage of cells that lose target protein expression (FACS). Treg cells WT or KO were then expanded by several rounds of stimulation with CD3/CD28 beads and IL-2.

As observed in FIG. 14, CD74-gene expression is efficiently abrogated in 40% of Tregs with the CRISPR/Cas9 KO technique.

To analyze the role of their candidate genes CD74 on human Treg biology, the inventors studied the viability, proliferation and phenotype (FACS expression of Treg-associated proteins: i.e. HLA-DR, Ki67, CD25, OX40 and 4-1BB).

The inventors observed that CD74 KO Tregs, compared to their WT counterparts showed defects in in vitro expansion as well as lower levels of Ki67 expression, and expressed lower levels of CD25, OX40, HLA-DR, and higher levels of 4-1BB (FIG. 15).

4. Validation that Functional Inhibition of CD74-Mediated Migration of Tregs could be Performed by Blocking its Co-Ligand MIF with a Small Molecule or an Anti-MIF Antibody.

The inventors have evaluated the co-expression of CD74 with MIF co-receptors at the surface of Tregs, and have observed that effectively, Tregs co-express CD74 with known MIF co-receptors, namely CXCR4, CXCR2 and CD44 (FIG. 16).

5. Study of the Suppressive Function of Genetically Modified Tregs by Comparing them with their WT Counterparts

Criss-cross experiments can be done using Tregs KO or WT for the candidate gene. For suppression tests, the inventors have set up two assays: classical suppression test of Tconv proliferation and modulation of co-stimulatory markers (CD86, CD80, CD40L, HLA-DR) in antigen presenting cells obtained from mice and/or allogenic donors.

TABLE 1
List of functional tumor-specific Treg markers identified in the application

| | | mRNA | mRNA | Protein | Up/ | Cell | | |
|---|---|---|---|---|---|---|---|---|
| | Human | accession | accession | accession | Down | Membrane | | |
| Gene name | Gene | number | number | number | regu- | (CM) | Cell TM | CellSurface |
| (symbol) | ID | (Ensembl) | (Refseq) | (Uniprot) | lated | Status | Status | Expression |
| CD74 | 972 | ENSG00000019582 | NM_001025158, | P04233 | + | CM | TM-EC | + |
| | | | NM_001025159, | | | | | |
| | | | NM_001364083, | | | | | |
| | | | NM_001364084, | | | | | |
| | | | NM_004355 | | | | | |
| VDR | 7421 | ENSG00000111424 | NM_000376, | F1D8P8 | + | NO | NA | |
| | | | NM_001017535, | P11473 | | | | |
| | | | NM_001017536, | | | | | |
| | | | NM_001364085 | | | | | |
| AC133644.2 | NA | ENSG00000280721 | | NA | + | NA | NA | |
| LINC02099 | 101929450 | ENSG00000253490 | | NA | + | NA | NA | |
| (Synonym: | | | | | | | | |
| AC145110.1) | | | | | | | | |
| ACADVL | 37 | ENSG00000072778 | NM_000018, | P49748 | + | M, NO | NA | |
| | | | NM_001033859, | B3KPA6 | | | | |
| | | | NM_001270447, | | | | | |
| | | | NM_001270448 | | | | | |
| ACSL4 | 2182 | ENSG00000068366 | NM_001318509, | O60488 | + | CM, NO | TM, NA | |
| | | | NM_001318510, | Q8TAF6 | | | | |
| | | | NM_004458, | | | | | |
| | | | NM_022977 | | | | | |
| ACTR1B | 10120 | ENSG00000115073 | NM_005735 | P42025 | + | NO | NA | |
| ADORA2A | 135 | ENSG00000128271 | NM_000675, | B3KVQ4 | + | CM | TM, TM, | + |
| | | | NM_001278497, | P29274 | | | EC | |
| | | | NM_001278498, | X5DNB4 | | | | |
| | | | NM_001278499, | A8K1F6 | | | | |
| | | | NM_001278500 | | | | | |
| ALDOA | 226 | ENSG00000149925 | NM_000034, | P04075 | + | NO | NA | |
| | | | NM_001127617, | V9HWN7 | | | | |
| | | | NM_001243175, | | | | | |
| | | | NM_001243177, | | | | | |
| | | | NM_001355562, | | | | | |
| | | | NM_001355563, | | | | | |
| | | | NM_001355564, | | | | | |
| | | | NM_001355565, | | | | | |
| | | | NM_184041, | | | | | |
| | | | NM_184043 | | | | | |
| ANXA6 | 309 | ENSG00000197043 | NM_001155, | A0A0S2Z2Z6, | + | NO | NA | |
| | | | NM_001193544, | P08133 | | | | |
| | | | NM_001363114, | | | | | |
| | | | NM_004033 | | | | | |
| ARF4 | 378 | ENSG00000168374 | NM_001660 | P18085 | + | M | NA | |
| ASB2 | 51676 | ENSG00000100628 | NM_001202429, | A0A024R6E7 | + | NO | NA | |
| | | | NM_016150 | Q96Q27 | | | | |
| ATIC | 471 | ENSG00000138363 | NM_004044 | P31939 | + | NO | NA | |
| | | | | V9HWH7 | | | | |
| ATP6V0C | 527 | ENSG00000185883 | NM_001198569, | P27449 | + | M | TM | |
| | | | NM_001694 | | | | | |
| BATF | 10538 | ENSG00000156127 | NM_006399 | Q16520 | + | NO | NA | |
| BCL2L1 | 598 | ENSG00000171552 | NM_001191, | Q07817, | + | M, NO | TM | |
| | | | NM_001317919, | Q5TE63, | | | | |
| | | | NM_001317920, | A0A0S2Z3C5 | | | | |
| | | | NM_001317921, | | | | | |
| | | | NM_001322239, | | | | | |
| | | | NM_001322240, | | | | | |
| | | | NM_001322242, | | | | | |
| | | | NM_138578 | | | | | |
| BCL3 | 602 | ENSG00000069399 | NM_005178 | P20749 | + | NO | NA | |
| | | | | B7Z3N9 | | | | |
| EPOP | 100170841 | ENSG00000273604 | NM_001130677 | A6NHQ4 | + | NO | NA | |
| (Synonym: | | | | | | | | |
| C17orf96) | | | | | | | | |
| C1orf112 | 55732 | ENSG00000000460 | NM_001320047, | A0A024R922, | + | NO | NA | |
| | | | NM_001320048, | Q9NSG2, | | | | |
| | | | NM_001320050, | B4E0A9, | | | | |
| | | | NM_001320051, | A0A1B0GV14, | | | | |
| | | | NM_001363739, | A0A1B0GUP7 | | | | |
| | | | NM_001366768, | | | | | |
| | | | NM_001366769, | | | | | |
| | | | NM_001366770, | | | | | |
| | | | NM_001366771, | | | | | |
| | | | NM_001366772, | | | | | |
| | | | NM_001366773, | | | | | |
| | | | NM_018186 | | | | | |
| CALR | 811 | ENSG00000179218 | NM_004343 | P27797, | + | Csurface, | NA | + |
| | | | | V9HW88 | | NO | | |
| CAMK1 | 8536 | ENSG00000134072 | NM_003656 | B0YIY3, | + | NO | NA | |
| | | | | Q14012 | | | | |
| CCL22 | 6367 | ENSG00000102962 | NM_002990 | O00626 | + | NO | NA | |
| CCND2 | 894 | ENSG00000118971 | NM_001759 | P30279 | + | M | NA | |
| CCR8 | 1237 | ENSG00000179934 | NM_005201 | P51685 | + | CM | TM-EC | + |
| CD4 | 920 | ENSG00000010610 | NM_000616, | B4DT49 | + | NO, CM | TM, | + |
| | | | NM_001195014, | P01730 | | | TM-EC | |
| | | | NM_001195015, | B0AZV7 | | | | |
| | | | NM_001195016 | | | | | |
| | | | NM_001195017, | | | | | |
| CD7 | 924 | ENSG00000173762 | NM_006137 | P09564 | + | M, NO | TM-EC, | + |
| | | | | Q29VG3 | | | TM | |
| | | | | J3QLC7 | | | | |
| CD80 | 941 | ENSG00000121594 | NM_005191 | A0N0P2 | + | NO, M | TM, | + |
| | | | | P33681 | | | TM-EC | |
| CD82 | 3732 | ENSG00000085117 | NM_001024844, | P27701 | + | CM | TM-EC | + |
| | | | NM_002231 | | | | | |
| CD83 | 9308 | ENSG00000112149 | NM_001040280, | Q01151 | + | M, NO | TM-EC, | + |
| | | | NM_001251901, | A0A087WX61 | | | TM | |
| | | | NM_004233 | | | | | |
| CDC37 | 11140 | ENSG00000105401 | NM_007065 | A0A024R7B7, | + | NO | NA | |
| | | | | Q16543 | | | | |
| CDKN1A | 1026 | ENSG00000124762 | NM_000389, | A0A024RCX5P38936 | + | NO | NA | |
| | | | NM_001220777, | | | | | |
| | | | NM_001220778, | | | | | |
| | | | NM_001291549, | | | | | |
| | | | NM_078467 | | | | | |
| CFLAR | 8837 | ENSG00000003402 | NM_001127183, | A0A024R3Z7O15519 | + | NO | NA | |
| | | | NM_001127184, | A0A024R3Y3 | | | | |
| | | | NM_001202515, | B4E361, | | | | |
| | | | NM_001202516, | E9PAP3 | | | | |
| | | | NM_001202517, | Q59F61 | | | | |
| | | | NM_001202518, | | | | | |
| | | | NM_001202519, | | | | | |
| | | | NM_001308042, | | | | | |
| | | | NM_001308043, | | | | | |
| | | | NM_001351590, | | | | | |
| | | | NM_001351591, | | | | | |
| | | | NM_001351592, | | | | | |
| | | | NM_001351593, | | | | | |
| | | | NM_001351594, | | | | | |
| | | | NM_003879 | | | | | |
| CISD3 | 284106 | ENSG00000277972 | NM_001136498 | P0C7P0 | + | NO | NA | |
| CRADD | 8738 | ENSG00000169372 | NM_001320099, | P78560, | + | NO | NA | |
| | | | NM_001320100, | Q53XL1, | | | | |
| | | | NM_001320101, | Q8IY43, | | | | |
| | | | NM_001330126, | B4DJT6, | | | | |
| | | | NM_003805 | F5H7C2, | | | | |
| | | | | F8VV49, | | | | |
| | | | | F8VVY5 | | | | |
| CSF1 | 1435 | ENSG00000184371 | NM_000757, | A0A024R0A1, | + | NO, CM | TM | + |
| | | | NM_172210, | P09603 | | | | |
| | | | NM_172211, | | | | | |
| | | | NM_172212 | | | | | |
| CSRNP1 | 64651 | ENSG00000144655 | NM_001320559, | A0A024R2N7, | + | NO | NA | |
| | | | NM_001320560, | Q96S65 | | | | |
| | | | NM_033027 | | | | | |
| CTLA4 | 1493 | ENSG00000163599 | NM_001037631, | P16410 | + | CM | TM-EC | + |
| | | | NM_005214 | | | | | |
| CTNNA1 | 1495 | ENSG00000044115 | NM_001290307, | G3XAM7, | + | NO, CM | NA | |
| | | | NM_001290309, | P35221, | | | | |
| | | | NM_001290310, | B4DKT9, | | | | |
| | | | NM_001290312, | B4DU00 | | | | |
| | | | NM_001323982, | | | | | |
| | | | NM_001323983, | | | | | |
| | | | NM_001323984, | | | | | |
| | | | NM_001323985, | | | | | |
| | | | NM_001323986, | | | | | |
| | | | NM_001323987, | | | | | |
| | | | NM_001323988, | | | | | |
| | | | NM_001323989, | | | | | |
| | | | NM_001323990, | | | | | |
| | | | NM_001323991, | | | | | |
| | | | NM_001323992, | | | | | |
| | | | NM_001323993, | | | | | |
| | | | NM_001323994, | | | | | |
| | | | NM_001323995, | | | | | |
| | | | NM_001323996, | | | | | |
| | | | NM_001323997, | | | | | |
| | | | NM_001323998, | | | | | |
| | | | NM_001323999, | | | | | |
| | | | NM_001324000, | | | | | |
| | | | NM_001324001, | | | | | |
| | | | NM_001324002, | | | | | |
| | | | NM_001324003, | | | | | |
| | | | NM_001324004, | | | | | |
| | | | NM_001324005, | | | | | |
| | | | NM_001324006, | | | | | |
| | | | NM_001324007, | | | | | |
| | | | NM_001324008, | | | | | |
| | | | NM_001324009, | | | | | |
| | | | NM_001324010, | | | | | |
| | | | NM_001324011, | | | | | |
| | | | NM_001324012, | | | | | |
| | | | NM_001324013, | | | | | |
| | | | NM_001903 | | | | | |
| CUL9 | 23113 | ENSG00000112659 | NM_015089 | Q8IWT3 | + | NO | NA | |
| CXCR3 | 2833 | ENSG00000186810 | NM_001142797, | P49682 | + | CM | TM-EC | + |
| | | | NM_001504 | | | | | |
| DNPH1 | 10591 | ENSG00000112667 | NM_006443, | O43598 | + | NO | NA | |
| | | | NM_199184 | | | | | |
| DUSP2 | 1844 | ENSG00000158050 | NM_004418 | Q05923 | + | NO | NA | |
| DUSP4 | 1846 | ENSG00000120875 | NM_001394, | Q13115 | + | NO | NA | |
| | | | NM_057158 | | | | | |
| EBI3 | 10148 | ENSG00000105246 | NM_005755 | Q14213 | + | NO | NA | |
| EED | 8726 | ENSG00000074266 | NM_001308007, | O75530, | + | NO | NA | |
| | | | NM_001330334, | E9PJK2 | | | | |
| | | | NM_003797, | | | | | |
| | | | NM_152991 | | | | | |
| EMD | 2010 | ENSG00000102119 | NM_000117 | P50402 | + | M | TM | |
| ETV7 | 51513 | ENSG00000010030 | NM_001207035, | Q9Y603 | + | NO | NA | |
| | | | NM_001207036, | | | | | |
| | | | NM_001207037, | | | | | |
| | | | NM_001207038, | | | | | |
| | | | NM_001207039, | | | | | |
| | | | NM_001207040, | | | | | |
| | | | NM_001207041, | | | | | |
| | | | NM_016135 | | | | | |
| F5 | 2153 | ENSG00000198734 | NM_000130 | P12259 | + | NO | NA | |
| FAM110A | 83541 | ENSG00000125898 | NM_001042353, | Q9BQ89 | + | NO | NA | |
| | | | NM_001289145, | | | | | |
| | | | NM_001289146, | | | | | |
| | | | NM_001289147, | | | | | |
| | | | NM_031424, | | | | | |
| | | | NM_207121 | | | | | |
| CIAO2B | 51647 | ENSG00000166595 | NM_016062 | Q9Y3D0 | + | NO | NA | |
| (Synonym: | | | | | | | | |
| FAM96B) | | | | | | | | |
| FLVCR2 | 55640 | ENSG00000119686 | NM_001195283, | Q9UPI3 | + | CM | TM | |
| | | | NM_017791 | | | | | |
| FOXP3 | 50943 | ENSG00000049768 | NM_001114377, | Q9BZS1, | + | NO | NA | |
| | | | NM_014009 | B7ZLG1 | | | | |
| GBP2 | 2634 | ENSG00000162645 | NM_004120 | P32456, | + | M, NO | NA | |
| | | | | Q8TCE5 | | | | |
| GCNT1 | 2650 | ENSG00000187210 | NM_001097633, | Q02742, | + | M, NO | TM, NA | |
| | | | NM_001097634, | Q86T81 | | | | |
| | | | NM_001097635, | | | | | |
| | | | NM_001097636, | | | | | |
| | | | NM_001490 | | | | | |
| GEM | 2669 | ENSG00000164949 | NM_005261, | A0A024R9F5, | + | NO, CM | NA | |
| | | | NM_181702 | P55040 | | | | |
| GIMAP4 | 55303 | ENSG00000133574 | NM_001363532, | A0A090N7X0, | − | NO | NA | |
| | | | NM_018326 | Q9NUV9 | | | | |
| GIMAP7 | 168537 | ENSG00000179144 | NM_153236 | A0A090N8P8, | − | NO | NA | |
| | | | | Q8NHV1 | | | | |
| GNA15 | 2769 | ENSG00000060558 | NM_002068 | P30679 | + | NO | NA | |
| GOT2 | 2806 | ENSG00000125166 | NM_001286220, | P00505 | + | CM | NA | |
| | | | NM_002080 | | | | | |
| GRINA | 2907 | ENSG00000178719 | NM_000837, | Q7Z429 | + | M | TM | |
| | | | NM_001009184 | | | | | |
| GRSF1 | 2926 | ENSG00000132463 | NM_001098477, | A0A024RD99, | + | NO | NA | |
| | | | NM_002092 | Q12849 | | | | |
| GTF2E2 | 2961 | ENSG00000197265 | NM_001348353, | P29084 | + | NO | NA | |
| | | | NM_002095 | | | | | |
| H3-3B | 3021 | ENSG00000132475 | NM_005324 | B2R4P9, | + | NO | NA | |
| (Synonym: | | | | P84243 | | | | |
| H3F3B) | | | | | | | | |
| HLA-B | 3106 | ENSG00000234745 | NM_005514 | E5FQ95, | + | NO, CM | TM, | + |
| | | | | P01889 | | | TM-EC | |
| HLA-DQA1 | 3117 | ENSG00000196735 | NM_002122 | A0A173ADG5, | + | NO, CM | TM, | + |
| | | | | P01909, | | | TM-EC, | |
| | | | | Q8MH44 | | | NA | |
| HLA-DRB5 | 3127 | ENSG00000198502 | NM_002125 | Q30154 | + | CM | TM-EC | + |
| HSP90B1 | 7184 | ENSG00000166598 | NM_003299 | P14625, | + | NO | NA | |
| | | | | V9HWP2 | | | | |
| HTATIP2 | 10553 | ENSG00000109854 | NM_001098520, | Q9BUP3 | + | NO | NA | |
| | | | NM_001098521, | | | | | |
| | | | NM_001098522, | | | | | |
| | | | NM_001098523, | | | | | |
| | | | NM_006410 | | | | | |
| HYOU1 | 10525 | ENSG00000149428 | NM_001130991, | B3KXH0, | + | NO | NA | |
| | | | NM_006389 | B7Z602, | | | | |
| | | | | B7Z766, | | | | |
| | | | | Q6IN67, | | | | |
| | | | | Q9Y4L1 | | | | |
| ICAM1 | 3383 | ENSG00000090339 | NM_000201 | P05362 | + | M | TM-EC | + |
| ICOS | 29851 | ENSG00000163600 | NM_012092 | Q53QY6, | + | NO, CM | TM, | + |
| | | | | Q9Y6W8 | | | TM-EC | |
| ID3 | 3399 | ENSG00000117318 | NM_002167 | Q02535 | + | NO | NA | |
| IGFLR1 | 79713 | ENSG00000126246 | NM_001346003, | K7EL86, | + | NO, CM | NA, | + |
| | | | NM_001346004, | Q9H665 | | | TM-EC | |
| | | | NM_001346005, | | | | | |
| | | | NM_001346006, | | | | | |
| | | | NM_024660 | | | | | |
| IL12RB2 | 3595 | ENSG00000081985 | NM_001258214, | B4DGA4, | + | NO, M | TM, | + |
| | | | NM_001258215, | Q99665, | | | TM-EC, | |
| | | | NM_001258216, | A0A0A0MTN7, | | | NA | |
| | | | NM_001319233, | B7ZB60 | | | | |
| | | | NM_001374259, | | | | | |
| | | | NM_001559 | | | | | |
| IL1R2 | 7850 | ENSG00000115590 | NM_001261419, | P27930 | + | CM | TM-EC | + |
| | | | NM_004633, | | | | | |
| | | | NM_173343 | | | | | |
| IL21R | 50615 | ENSG00000103522 | NM_021798, | Q9HBE5 | + | M | TM-EC | + |
| | | | NM_181078, | | | | | |
| | | | NM_181079 | | | | | |
| IL2RA | 3559 | ENSG00000134460 | NM_000417, | P01589, | + | M, NO | TM, | + |
| | | | NM_001308242, | Q5W005 | | | EC-TM | |
| | | | NM_001308243 | | | | | |
| IL2RB | 3560 | ENSG00000100385 | NM_000878, | P14784 | + | CS | TM-EC | + |
| | | | NM_001346222, | | | | | |
| | | | NM_001346223 | | | | | |
| IL2RG | 3561 | ENSG00000147168 | NM_000206 | P31785 | + | CS | TM-EC | + |
| INSIG1 | 3638 | ENSG00000186480 | NM_001346590, | A4D2M9, | + | M, NO | TM | |
| | | | NM_001346591, | O15503, | | | | |
| | | | NM_001346592, | A0A024RD68, | | | | |
| | | | NM_001346593, | F5H6P3 | | | | |
| | | | NM_001346594, | | | | | |
| | | | NM_005542, | | | | | |
| | | | NM_198336, | | | | | |
| | | | NM_198337 | | | | | |
| IRF5 | 3663 | ENSG00000128604 | NM_001098627, | Q13568, | + | NO | NA | |
| | | | NM_001098629, | B7Z1M2, | | | | |
| | | | NM_001098630, | C9JAU6, | | | | |
| | | | NM_001242452, | A0A0G2USB5 | | | | |
| | | | NM_001347928, | | | | | |
| | | | NM_001364314, | | | | | |
| | | | NM_032643 | | | | | |
| KCNN4 | 3783 | ENSG00000104783 | NM_002250 | O15554 | + | CM | TM | |
| KDM6B | 23135 | ENSG00000132510 | NM_001080424, | O15054 | + | NO | NA | |
| | | | NM_001348716 | | | | | |
| LASP1 | 3927 | ENSG00000002834 | NM_001271608, | B4DIC4, | + | NO | NA | |
| | | | NM_006148 | Q14847, | | | | |
| | | | | A0A024R1S8 | | | | |
| LGALS3 | 3958 | ENSG00000131981 | NM_001177388, | A0A024R693, | + | NO | NA | |
| | | | NM_001357678, | P17931 | | | | |
| | | | NM_002306 | | | | | |
| LINC01588 | 283551 | ENSG00000214900 | NM_001012706 | NA | + | NA | NA | |
| LMNA | 4000 | ENSG00000160789 | NM_001257374, | P02545 | + | NO | NA | |
| | | | NM_001282624, | Q5TCI8 | | | | |
| | | | NM_001282625, | | | | | |
| | | | NM_001282626, | | | | | |
| | | | NM_005572, | | | | | |
| | | | NM_170707, | | | | | |
| | | | NM_170708 | | | | | |
| LRRC32 | 2615 | ENSG00000137507 | NM_001128922, | A0A024R5J7, | + | NO, CS | TM, | + |
| | | | NM_001370187, | Q14392 | | | TM-EC | |
| | | | NM_001370188, | | | | | |
| | | | NM_001370189, | | | | | |
| | | | NM_001370190, | | | | | |
| | | | NM_001370191, | | | | | |
| | | | NM_005512 | | | | | |
| LSP1 | 4046 | ENSG00000130592 | NM_001013253, | P33241, | + | CM, NO | NA | |
| | | | NM_001013254, | A8K7L8 | | | | |
| | | | NM_001013255, | | | | | |
| | | | NM_001242932, | | | | | |
| | | | NM_001289005, | | | | | |
| | | | NM_002339 | | | | | |
| LTA | 4049 | ENSG00000226979 | NM_000595, | P01374, | + | M, NO | NA | |
| | | | NM_001159740 | Q5STV3 | | | | |
| LYPLA2 | 11313 | ENSG00000011009 | NM_007260 | A0A140VJC9 | + | NO | NA | |
| | | | | O95372 | | | | |
| MAP1LC3A | 84557 | ENSG00000101460 | NM_032514, | Q9H492 | + | M | NA | |
| | | | NM_181509 | | | | | |
| MAP2K3 | 5606 | ENSG00000034152 | NM_001316332, | P46734, | + | NO | NA | |
| | | | NM_002756, | Q6FI23 | | | | |
| | | | NM_145109, | | | | | |
| | | | NM_145110 | | | | | |
| MFSD10 | 10227 | ENSG00000109736 | NM_001120, | Q14728, | + | M, NO | TMe | |
| | | | NM_001146069, | D6RE79 | | | | |
| | | | NM_001363679 | | | | | |
| MICAL2 | 9645 | ENSG00000133816 | NM_001282663, | O94851 | + | NO | NA | |
| | | | NM_001282664, | | | | | |
| | | | NM_001282665, | | | | | |
| | | | NM_001282666, | | | | | |
| | | | NM_001282667, | | | | | |
| | | | NM_001282668, | | | | | |
| | | | NM_001346292, | | | | | |
| | | | NM_001346293, | | | | | |
| | | | NM_001346294, | | | | | |
| | | | NM_001346294, | | | | | |
| | | | NM_001346295, | | | | | |
| | | | NM_001346296, | | | | | |
| | | | NM_001346297, | | | | | |
| | | | NM_001346298, | | | | | |
| | | | NM_001346299, | | | | | |
| | | | NM_014632 | | | | | |
| MRPL38 | 64978 | ENSG00000204316 | NM_032478 | Q96DV4 | + | NO | NA | |
| MT-ND4 | 4538 | ENSG000000198886 | | H9EC08, | − | M | TM | |
| (Synonym: | | | | P03905 | | | | |
| ND4) | | | | | | | | |
| MVP | 9961 | ENSG00000013364 | NM_001293204, | Q14764, | + | NO | NA | |
| | | | NM_001293205, | X5DNU0, | | | | |
| | | | NM_005115, | X5D7K9, | | | | |
| | | | NM_017458 | X5D2M8 | | | | |
| NAMPT | 10135 | ENSG00000105835 | NM_005746, | A0A024R718, | + | NO | NA | |
| | | | NM_182790 | P43490 | | | | |
| NDFIP2 | 54602 | ENSG00000102471 | NM_001161407, | B4DGY6, | + | NO, M | TM, | + |
| | | | NM_019080 | Q9NV92 | | | TM-EC | |
| NFKB2 | 4791 | ENSG00000077150 | NM_001077494, | Q00653 | + | NO | NA | |
| | | | NM_001261403, | | | | | |
| | | | NM_001288724, | | | | | |
| | | | NM_001322934, | | | | | |
| | | | NM_001322935, | | | | | |
| | | | NM_002502 | | | | | |
| NFKBID | 84807 | ENSG00000167604 | NM_001321831, | Q8NI38 | + | NO | NA | |
| | | | NM_001365705, | | | | | |
| | | | NM_001365706, | | | | | |
| | | | NM_032721, | | | | | |
| | | | NM_139239 | | | | | |
| NINJ1 | 4814 | ENSG00000131669 | NM_004148 | Q92982 | + | M | TM-EC | + |
| NTRK1 | 4914 | ENSG00000198400 | NM_001007792, | P04629, | + | CM, NO | TM-EC, | + |
| | | | NM_001012331, | X5DR71 | | | TM | |
| | | | NM_002529 | | | | | |
| PARVB | 29780 | ENSG00000188677 | NM_001003828, | Q9HBI1, | + | CM, NO | NA | |
| | | | NM_001243385, | A0A087WZB5 | | | | |
| | | | NM_001243386, | | | | | |
| | | | NM_013327 | | | | | |
| PCBP1 | 5093 | ENSG00000169564 | NM_006196 | Q15365, | + | NO | NA | |
| | | | | Q53SS8 | | | | |
| PDGFA | 5154 | ENSG00000197461 | NM_002607, | P04085 | + | NO | NA | |
| | | | NM_033023 | | | | | |
| PFKP | 5214 | ENSG00000067057 | NM_001242339, | Q01813 | + | NO | NA | |
| | | | NM_001323067, | | | | | |
| | | | NM_001323068, | | | | | |
| | | | NM_001323069, | | | | | |
| | | | NM_001323070, | | | | | |
| | | | NM_001323071, | | | | | |
| | | | NM_001323072, | | | | | |
| | | | NM_001323073, | | | | | |
| | | | NM_001323074, | | | | | |
| | | | NM_001345944, | | | | | |
| | | | NM_002627 | | | | | |
| PGAM1 | 5223 | ENSG00000171314 | NM_001317079, | B7Z9E5, | + | NO | NA | |
| | | | NM_002629 | Q6FHU2, | | | | |
| | | | | P18669 | | | | |
| PIM3 | 415116 | ENSG00000198355 | NM_001001852 | Q86V86 | + | NO | NA | |
| PKM | 5315 | ENSG00000067225 | NM_001206796, | P14618, | + | NO | NA | |
| | | | NM_001206797, | B4DNK4, | | | | |
| | | | NM_001206798, | V9HWB8, | | | | |
| | | | NM_001206799, | A0A024R5Z9 | | | | |
| | | | NM_001316318, | | | | | |
| | | | NM_002654, | | | | | |
| | | | NM_182470, | | | | | |
| | | | NM_182471 | | | | | |
| PLAGL2 | 5326 | ENSG00000126003 | NM_002657 | Q9UPG8 | + | NO | NA | |
| POU2F2 | 5452 | ENSG00000028277 | NM_001207025, | P09086, | + | NO | NA | |
| | | | NM_001207026, | B5ME60, | | | | |
| | | | NM_001247994, | H0YLL6 | | | | |
| | | | NM_002698 | | | | | |
| PPM1M | 132160 | ENSG00000164088 | NM_001122870, | Q96MI6, | + | NO | NA | |
| | | | NM_144641 | B7XGB9 | | | | |
| PPP2R5C | 5527 | ENSG00000078304 | NM_001161725, | Q13362 | − | NO | NA | |
| | | | NM_001161726, | | | | | |
| | | | NM_001352912, | | | | | |
| | | | NM_001352913, | | | | | |
| | | | NM_001352914, | | | | | |
| | | | NM_001352915, | | | | | |
| | | | NM_001352916, | | | | | |
| | | | NM_002719, | | | | | |
| | | | NM_178586, | | | | | |
| | | | NM_178587, | | | | | |
| | | | NM_178588 | | | | | |
| PRDX5 | 25824 | ENSG00000126432 | NM_001358511, | P30044 | + | NO | NA | |
| | | | NM_001358516, | | | | | |
| | | | NM_012094, | | | | | |
| | | | NM_181651, | | | | | |
| | | | NM_181652 | | | | | |
| CAVIN3 | 112464 | ENSG00000170955 | NM_145040 | Q969G5 | + | Membrane | NA | |
| (Synonym: | | | | | | | | |
| PRKCDBP) | | | | | | | | |
| PRNP | 5621 | ENSG00000171867 | NM_000311, | P04156, | + | CM, M | NA, | |
| | | | NM_001080121, | Q53YK7, | | | TM | |
| | | | NM_001080122, | F7VJQ1 | | | | |
| | | | NM_001080123, | | | | | |
| | | | NM_001271561, | | | | | |
| | | | NM_183079 | | | | | |
| PSMA6 | 5687 | ENSG00000100902 | NM_001282232, | P60900, | + | NO | NA | |
| | | | NM_001282233, | A0A140VK44 | | | | |
| | | | NM_001282234, | | | | | |
| | | | NM_002791 | | | | | |
| PTP4A3 | 11156 | ENSG00000184489 | NM_007079, | O75365 | + | CM | NA | |
| | | | NM_032611 | | | | | |
| PTPN6 | 5777 | ENSG00000111679 | NM_002831, | P29350, | + | NO | NA | |
| | | | NM_080548, | Q53XS4 | | | | |
| | | | NM_080549 | | | | | |
| PTPRCAP | 5790 | ENSG00000213402 | NM_005608 | Q14761 | + | M | TM | |
| RAC2 | 5880 | ENSG00000128340 | NM_002872 | A0A024R1P2, | + | NO | NA | |
| | | | | P15153, | | | | |
| | | | | V9H0H7 | | | | |
| RARA | 5914 | ENSG00000131759 | NM_000964, | P10276, | + | NO | NA | |
| | | | NM_001024809, | Q6I9R7, | | | | |
| | | | NM_001033603, | F1D8N9, | | | | |
| | | | NM_001145301, | A8K840, | | | | |
| | | | NM_001145302 | A8MUP8 | | | | |
| RASSF5 | 83593 | ENSG00000266094 | NM_031437, | A8K5F3, | + | NO | NA | |
| | | | NM_182663, | Q8WWW0 | | | | |
| | | | NM_182664, | | | | | |
| | | | NM_182665 | | | | | |
| RELB | 5971 | ENSG00000104856 | NM_006509 | Q01201, | + | NO | NA | |
| | | | | D6R992 | | | | |
| RHOG | 391 | ENSG00000177105 | NM_001665 | P84095, | + | CM, NO | NA | |
| | | | | Q6ICQ8 | | | | |
| RILPL2 | 196383 | ENSG00000150977 | NM_145058 | Q969X0 | + | NO | NA | |
| RNF187 | 149603 | ENSG00000168159 | NM_001010858 | Q5TA31 | + | NO | NA | |
| RNH1 | 6050 | ENSG00000023191 | NM_002939, | A0A140VJT8, | + | NO | NA | |
| | | | NM_203383, | P13489 | | | | |
| | | | NM_203384, | | | | | |
| | | | NM_203385, | | | | | |
| | | | NM_203386, | | | | | |
| | | | NM_203387, | | | | | |
| | | | NM_203388, | | | | | |
| | | | NM_203389 | | | | | |
| RP11- | NA | ENSG00000257924 | | NA | + | NA | NA | |
| 493L12.5 | | | | | | | | |
| SDC4 | 6385 | ENSG00000124145 | NM_002999 | P31431, | + | M | Trans- | + |
| | | | | B4E1S6 | | | membrane- | |
| | | | | | | | ExtraCell, | |
| | | | | | | | Trans- | |
| | | | | | | | membrane | |
| SDF4 | 51150 | ENSG00000078808 | NM_016176, | A0A024R084, | + | NO, CM | NA | |
| | | | NM_016547 | Q9BRK5, | | | | |
| | | | | A0A024R0A9 | | | | |
| SERPINB9 | 5272 | ENSG00000170542 | NM_004155 | A0A024QZT4, | + | NO | NA | |
| | | | | P50453 | | | | |
| SERTAD1 | 29950 | ENSG00000197019 | NM_013376 | Q53GC0, | + | NO | NA | |
| | | | | Q9UHV2 | | | | |
| SGPP2 | 130367 | ENSG00000163082 | NM_001320833, | Q8IWX5 | + | M | TM | |
| | | | NM_001320834, | | | | | |
| | | | NM_152386 | | | | | |
| SH2D2A | 9047 | ENSG00000027869 | NM_001161441, | Q5UBZ2, | + | NO | NA | |
| | | | NM_001161442, | Q9NP31, | | | | |
| | | | NM_001161443, | Q5UBZ3 | | | | |
| | | | NM_001161444, | | | | | |
| | | | NM_003975 | | | | | |
| SH3BP1 | 23616 | ENSG00000100092 | NM_001350055, | Q9Y3L3 | + | NO | NA | |
| | | | NM_018957 | | | | | |
| SLC1A5 | 6510 | ENSG00000105281 | NM_001145144, | Q15758, | + | CM, M | TM-EC, | + |
| | | | NM_001145145, | Q59ES3 | | | TM | |
| | | | NM_005628 | | | | | |
| SLC25A4 | 291 | ENSG00000151729 | NM_001151 | A0A0S2Z3H3 | + | NO, M | TM | |
| | | | | P12235 | | | | |
| SLC3A2 | 6520 | ENSG00000168003 | NM_001012661, | J3KPF3, | + | NO, CM | TM-EC | + |
| | | | NM_001012662, | P08195 | | | | |
| | | | NM_001012663, | | | | | |
| | | | NM_001012664, | | | | | |
| | | | NM_001013251, | | | | | |
| | | | NM_002394 | | | | | |
| SLC7A5 | 8140 | ENSG00000103257 | NM_003486 | Q01650 | + | CM | TM-EC | + |
| SLCO4A1 | 28231 | ENSG00000101187 | NM_016354 | Q96BD0 | + | CM | TM-EC | + |
| SNX9 | 51429 | ENSG00000130340 | NM_016224 | Q9Y5X1 | + | CM | NA | |
| SQSTM1 | 8878 | ENSG00000161011 | NM_001142298, | Q13501 | + | NO | NA | |
| | | | NM_001142299, | | | | | |
| | | | NM_003900 | | | | | |
| SRA1 | 10011 | ENSG00000213523 | NM_001035235, | Q9HD15 | + | NO | NA | |
| | | | NM_001253764 | | | | | |
| ZNRD2 | 10534 | ENSG00000173465 | NM_001303024, | G3V1B8, | + | NO | NA | |
| (Synonym: | | | NM_006396 | O60232 | | | | |
| SSSCA1) | | | | | | | | |
| STAT5A | 6776 | ENSG00000126561 | NM_001288718, | A8K6I5, | + | NO | NA | |
| | | | NM_001288719, | P42229, | | | | |
| | | | NM_001288720, | Q59GY7, | | | | |
| | | | NM_003152 | K7EK35 | | | | |
| SYNGR2 | 9144 | ENSG00000108639 | NM_001320523, | O43760, | + | M | TM | |
| | | | NM_001363778, | A0A024R8T9 | | | | |
| | | | NM_004710 | | | | | |
| TMPRSS6 | 164656 | ENSG00000187045 | NM_001289000, | Q8IU80 | + | CM | TM-EC | + |
| | | | NM_001289001, | | | | | |
| | | | NM_153609 | | | | | |
| TNFRSF18 | 8784 | ENSG00000186891 | NM_004195, | Q9Y5U5, | + | CM, NO | TM-EC, | + |
| | | | NM_148901, | A0A0R7FDM1 | | | NA | |
| | | | NM_148902 | | | | | |
| TNFRSF1B | 7133 | ENSG00000028137 | NM_001066 | P20333 | + | CM | TM-EC | + |
| TNFRSF4 | 7293 | ENSG00000186827 | NM_003327 | P43489 | + | M | TM-EC | + |
| TNFRSF8 | 943 | ENSG00000120949 | NM_001243, | A5D8T4, | + | NO, CM | NA, | + |
| | | | NM_001281430, | P28908 | | | TM-EC | |
| | | | NM_152942 | | | | | |
| TNFRSF9 | 3604 | ENSG00000049249 | NM_001561 | Q07011 | + | M | TM-EC | + |
| TNIP1 | 10318 | ENSG00000145901 | NM_001252385, | A0A0A0MRZ4, | + | NO | NA | |
| | | | NM_001252386, | B7Z8K2, | | | | |
| | | | NM_001252390, | Q15025, | | | | |
| | | | NM_001252391, | A8K4N4 | | | | |
| | | | NM_001252392, | | | | | |
| | | | NM_001252393, | | | | | |
| | | | NM_001258454, | | | | | |
| | | | NM_001258455, | | | | | |
| | | | NM_001258456, | | | | | |
| | | | NM_001364486, | | | | | |
| | | | NM_001364487, | | | | | |
| | | | NM_006058 | | | | | |
| TNIP2 | 79155 | ENSG00000168884 | NM_001161527, | Q8NFZ5, | + | NO | NA | |
| | | | NM_001292016, | D6RGJ2 | | | | |
| | | | NM_024309 | | | | | |
| TNIP3 | 79931 | ENSG00000050730 | NM_001128843, | Q96KP6 | + | NO | NA | |
| | | | NM_001244764, | | | | | |
| | | | NM_024873 | | | | | |
| TNS3 | 64759 | ENSG00000136205 | NM_022748 | Q68CZ2 | + | NO | NA | |
| TRAF1 | 7185 | ENSG00000056558 | NM_001190945, | Q13077 | + | NO | NA | |
| | | | NM_001190947, | | | | | |
| | | | NM_005658 | | | | | |
| TRAF4 | 9618 | ENSG00000076604 | NM_004295, | A0A024QZ59, | + | NO, CM | NA | |
| | | | NM_145751 | Q9BUZ4, | | | | |
| | | | | A0A024QZ19 | | | | |
| TSPAN13 | 27075 | ENSG00000106537 | NM_014399 | O95857, | + | M, NO | TM-EC, | + |
| | | | | Q6FGK0 | | | TM | |
| TSPAN17 | 26262 | ENSG00000048140 | NM_001006616, | Q96FV3, | + | M | TM-EC, | + |
| | | | NM_001366491, | J3KNG2, | | | TM | |
| | | | NM_001366492, | A0A024R7Q6 | | | | |
| | | | NM_012171, | | | | | |
| | | | NM_130465 | | | | | |
| TYMP | 1890 | ENSG00000025708 | NM_001113755, | B2RBL3, | + | NO | NA | |
| | | | NM_001113756, | E5KRG5, | | | | |
| | | | NM_001257988, | P19971 | | | | |
| | | | NM_001257989, | | | | | |
| | | | NM_001953 | | | | | |
| UBA1 | 7317 | ENSG00000130985 | NM_003334, | A0A024R1A3, | + | NO | NA | |
| | | | NM_153280 | P22314 | | | | |
| USP12 | 219333 | ENSG00000152484 | NM_182488 | O75317 | + | NO | NA | |
| VASP | 7408 | ENSG00000125753 | NM_001008736, | A0A024R0V4, | + | NO, CM | NA | |
| | | | NM_003370 | P50552 | | | | |
| VCP | 7415 | ENSG00000165280 | NM_001354927, | P55072, | + | NO | NA | |
| | | | NM_001354928, | V9HW80 | | | | |
| | | | NM_007126 | | | | | |
| WARS1 | 7453 | ENSG00000140105 | NM_004184, | A0A024R6K8, | + | NO | NA | |
| (Synonym: | | | NM_173701, | P23381 | | | | |
| WARS) | | | NM_213645, | | | | | |
| | | | NM_213646 | | | | | |
| WNT10A | 80326 | ENSG00000135925 | NM_025216 | Q9GZT5 | + | NO | NA | |
| XIRP1 | 165904 | ENSG00000168334 | NM_001198621, | Q702N8 | + | NO | NA | |
| | | | NM_001351377, | | | | | |
| | | | NM_194293 | | | | | |
| XXbac- | NA | ENSG00000263020 | | NA | + | NA | NA | |
| BPG32J3.22 | | | | | | | | |
| ZBTB32 | 27033 | ENSG00000011590 | NM_001316902, | Q9Y2Y4 | + | NO | NA | |
| | | | NM_001316903, | | | | | |
| | | | NM_014383 | | | | | |
| ZBTB7B | 51043 | ENSG00000160685 | NM_001252406, | O15156 | + | NO | NA | |
| | | | NM_001256455 | | | | | |
| ZC3H12A | 80149 | ENSG00000163874 | NM_001323550, | Q5D1E8 | + | M | NA | |
| | | | NM_001323551, | | | | | |
| | | | NM_025079 | | | | | |
| ZDHHC18 | 84243 | ENSG00000204160 | NM_032283 | Q9NUE0 | + | M | TM | |
| ZFP36 | 7538 | ENSG00000128016 | NM_003407 | M0QY76, | + | NO | NA | |
| | | | | P26651 | | | | |
| ZFP36L1 | 677 | ENSG00000185650 | NM_001244698, | A0A024R658, | + | NO | NA | |
| | | | NM_001244701, | B3KNA8, | | | | |
| | | | NM_004926 | Q07352 | | | | |

Upregulated (+); Downregulated (−);
CM: Cell membrane;
M: Membrane;
CS: Cell Surface
TM: Transmembrane;
EC: Extracellular;
NA: Not Applicable

TABLE 2
List of functional tumor-specific Treg markers identified in the application not listed in Table 1 (identified
upon STEP7 of Identification and ranking of tumor-specific Treg markers for therapeutic purpose)

| | | mRNA | mRNA | protein | | Cell | | |
|---|---|---|---|---|---|---|---|---|
| Gene | | accession | accession | accession | Up/ | Membrane | Cell | Cell |
| name | Human | number | number | number | Down- | (CM) | TM | Surface |
| (symbol) | GeneID | (Ensembl) | (RefSeq) | (Uniprot) | regulated | Status | STATUS | Expression |
| CD177 | 57126 | ENSG00000204936 | NM_020406 | A0A087WVM2 | + | CM | NA | + (GPI- |
| | | | | Q8N6Q3 | | | | anchored) |
| ENTPD1 | 953 | ENSG00000138185 | NM_001098175 | P49961 | + | CM | TM- | + |
| | | | NM_001164178 | | | | EC | |
| | | | NM_001164179 | | | | | |
| | | | NM_001164181 | | | | | |
| | | | NM_001164182 | | | | | |
| | | | NM_001164183 | | | | | |
| | | | NM_001312654 | | | | | |
| | | | NM_001320916 | | | | | |
| | | | NM_001776 | | | | | |
| HAVCR2 | 84868 | ENSG00000135077 | NM_032782 | Q8TDQ0 | + | CM | TM- | + |
| | | | | | | | EC | |
| CCR4 | 1233 | ENSG00000183813 | NM_005508 | A0N0Q1 | + | CM | TM- | + |
| | | | | P51679 | | | EC | |

### REFERENCES

Abbas, A. R., Wolslegel, K., Seshasayee, D., Modrusan, Z., Clark, H. F., 2009. Deconvolution of blood microarray data identifies cellular activation patterns in systemic lupus erythematosus. PLoS One. 2009 Jul. 1; 4(7):e6098. https://doi.org/10.1371/journal.pone.0006098.

Alonso, R., Flament, H., Lemoine, S., Sedlik, C., Bottasso, E., Peguillet, I., Premel, V., Denizeau, J., Salou, M., Darbois, A., Núñez, N. G., Salomon, B., Gross, D., Piaggio, E., Lantz, O., 2018. Induction of anergic or regulatory tumor-specific CD4+ T cells in the tumor-draining lymph node. Nat. Commun. 9, 2113. https://doi.org/10.1038/s41467-018-04524-x.

Azizi, E., Carr, A. J., Plitas, G., Cornish, A. E., Konopacki, C., Prabhakaran, S., Nainys, J., Wu, K., Kiseliovas, V., Setty, M., Choi, K., Fromme, R. M., Dao, P., McKenney, P. T., Wasti, R. C., Kadaveru, K., Mazutis, L., Rudensky, A. Y., Pe'er, D., 2018. Single-Cell Map of Diverse Immune Phenotypes in the Breast Tumor Microenvironment. Cell 174, 1293-1308.e36. https://doi.org/10.1016/j.cell.2018.05.060.

Barsheshet, Y., Wildbaum, G., Levy, E., Vitenshtein, A., Akinseye, C., Griggs, J., Lira, S. A., Karin, N., 2017. CCR8+ FOXp3+ Treg cells as master drivers of immune regulation. Proc. Natl. Acad. Sci. 201621280. https://doi.org/10.1073/pnas.1621280114.

Bates, G. J., Fox, S. B., Han, C., Leek, R. D., Garcia, J. F., Harris, A. L., Banham, A. H., 2006. Quantification of Regulatory T Cells Enables the Identification of High-Risk Breast Cancer Patients and Those at Risk of Late Relapse. J. Clin. Oncol. 24, 5373-5380. https://doi.org/10.1200/JC0.2006.05.9584.

Bluestone, J. A., Buckner, J. H., Fitch, M., Gitelman, S. E., Gupta, S., Hellerstein, M. K., Herold, K. C., Lares, A., Lee, M. R., Li, K., Liu, W., Long, S. A., Masiello, L. M., Nguyen, V., Putnam, A. L., Rieck, M., Sayre, P. H., Tang, Q., 2015. Type 1 diabetes immunotherapy using polyclonal regulatory T cells. Sci. Transl. Med. 7, 315ra189-315ra189. https://doi.org/10.1126/scitranslmed.aad4134.

Burzyn, D., Benoist, C., Mathis, D., 2013. REGULATORY T CELLS IN NON-LYMPHOID TISSUES. Nat. Immunol. 14, 1007-1013. https://doi.org/10.1038/ni.2683.

Butler, A., Hoffman, P., Smibert, P., Papalexi, E., Satija, R., 2018. Integrating single-cell transcriptomic data across different conditions, technologies, and species. Nat Biotechnol. 2018 June; 36(5): 411-420. https://doi.org/10.1038/nbt.4096.

Campbell, D. J., Koch, M. A., 2011. Phenotypical and functional specialization of FOXP3+ regulatory T cells. Nat. Rev. Immunol. 11, 119-130. https://doi.org/10.1038/nri2916.

Cantorna, M. T., Snyder, L., Lin, Y.-D., Yang, L., 2015. Vitamin D and 1.25(OH)2D Regulation of T cells. Nutrients 7, 3011-3021. https://doi.org/10.3390/nu7043011.

Caudana, P., Núñez, N. G., Rochere, P. D. L., Pinto, A., Denizeau, J., Alonso, R., Niborski, L. L., Lantz, O., Sedlik, C., Piaggio, E., 2019. IL2/Anti-IL2 Complex Combined with CTLA-4, But Not PD-1, Blockade Rescues Antitumor NK Cell Function by Regulatory T-cell Modulation. Cancer Immunol. Res. 7, 443-457. https://doi.org/10.1158/2326-6066.CIR-18-0697.

Chambers, E. S., Hawrylowicz, C. M., 2011. The impact of vitamin D on regulatory T cells. Curr. Allergy Asthma Rep. 11, 29-36. https://doi.org/10.1007/s11882-010-0161-8.

Chao, J. L., Savage, P. A., 2018. Unlocking the Complexities of Tumor-Associated Regulatory T Cells. J. Immunol. 200, 415-421. https://doi.org/10.4049/jimmuno1.1701188.Chaudhary, B., Elkord, E., 2016. Regulatory T Cells in the Tumor Microenvironment and Cancer Progression: Role and Therapeutic Targeting. Vaccines 4. https://doi.org/10.3390/vaccines4030028.

Chaudhry, A., Rudra, D., Treuting, P., Samstein, R. M., Liang, Y., Kas, A., Rudensky, A. Y., 2009. CD4+ Regulatory T Cells Control TH17 Responses in a Stat3-Dependent Manner. Science 326, 986-991. https://doi.org/10.1126/science.1172702.

Chen, D. S., Mellman, I., 2013. Oncology Meets Immunology: The Cancer-Immunity Cycle. Immunity 39, 1-10. https://doi.org/10.1016/j.immuni.2013.07.012.

Chung, W., Eum, H. H., Lee, H. O., Lee, K. M., Lee, H. B., Kim, K. T., Ryu, H. S., Kim, S., Lee, J. E., Park, Y. H., Kan, Z., Han, W., Park, W. Y., 2017. Single-cell RNA-seq enables comprehensive tumour and immune cell profiling in primary breast cancer. Nat Commun. 2017 May 5; 8:15081. https://doi.org/10.1038/ncomms15081.

Churlaud, G., Jimenez, V., Ruberte, J., Amadoudji Zin, M., Fourcade, G., Gottrand, G., Casana, E., Lambrecht, B., Bellier, B., Piaggio, E., Bosch, F., Klatzmann, D., 2014. Sustained stimulation and expansion of Tregs by IL2 control autoimmunity without impairing immune responses to infection, vaccination and cancer. Clin. Immunol. Orlando Fla. 151, 114-126. https://doi.org/10.1016/j.clim.2014.02.003.

De Simone, M., Arrigoni, A., Rossetti, G., Gruarin, P., Ranzani, V., Politano, C., Bonnal, R. J. P., Provasi, E., Sarnicola, M. L., Panzeri, I., Moro, M., Crosti, M., Mazzara, S., Vaira, V., Bosari, S., Palleschi, A., Santambrogio, L., Bovo, G., Zucchini, N., Totis, M., Gianotti, L., Cesana, G., Perego, R. A., Maroni, N., Pisani Ceretti, A., Opocher, E., De Francesco, R., Geginat, J., Stunnenberg, H. G., Abrignani, S., Pagani, M., 2016. Transcriptional Landscape of Human Tissue Lymphocytes Unveils Uniqueness of Tumor-Infiltrating T Regulatory Cells. Immunity 45, 1135-1147. https://doi.org/10.1016/j.immuni.2016.10.021.

Dwarakanath, B. S., Farooque, A., Gupta, S., 2018. Targeting regulatory T cells for improving cancer therapy: Challenges and prospects. Cancer Rep. 1, e21105. https://doi.org/10.1002/cnr2.1105.

Expression of CCR8 in cancer—Summary—The Human Protein Atlas [WWW Document], n.d. URL https://www.proteinatlas.org/ENSG00000179934-CCR8/pathology (accessed 4.24.19).

Faghih, Z., Erfani, N., Haghshenas, M. R., Safaei, A., Talei, A.-R., Ghaderi, A., 2014. Immune profiles of CD4+ lymphocyte subsets in breast cancer tumor draining lymph nodes. Immunol. Lett. 158, 57-65. https://doi.org/10.1016/j.imlet.2013.11.021.

Feuerer, M., Hill, J. A., Mathis, D., Benoist, C., 2009. Foxp3+ regulatory T cells: differentiation, specification, subphenotypes. Nat. Immunol. 10, 689-695. https://doi.org/10.1038/ni.1760.

Finak, G., McDavid, A., Yajima, M., Deng, J., Gersuk, V., Shalek, A. K., Slichter, C. K., Miller, H. W., McElrath, M. J., Prlic, M., Linsley, P. S., Gottardo, R., 2015. MAST: a flexible statistical framework for assessing transcriptional changes and characterizing heterogeneity in single-cell RNA sequencing data. Genome Biol. 2015 Dec. 10; 16:278. https://doi.org/10.1186/s13059-015-0844-5.

Fontenot, J. D., Gavin, M. A., Rudensky, A. Y., 2003. Foxp3 programs the development and function of CD4+CD25+ regulatory T cells. Nat. Immunol. 4, 330-336. https://doi.org/10.1038/ni904.

Gaidot, A., Landau, D. A., Martin, G. H., Bonduelle, O., Grinberg-Bleyer, Y., Matheoud, D., Grégoire, S., Baillou, C., Combadiére, B., Piaggio, E., Cohen, J. L., 2011. Immune reconstitution is preserved in hematopoietic stem cell transplantation coadministered with regulatory T cells for GVHD prevention. Blood 117, 2975-2983. https://doi.org/10.1182/blood-2010-08-299974.

Grinberg-Bleyer, Y., Saadoun, D., Baeyens, A., Billiard, F., Goldstein, J. D., Gregoire, S., Martin, G. H., Elhage, R., Derian, N., Carpentier, W., Marodon, G., Klatzmann, D., Piaggio, E., Salomon, B. L., 2010. Pathogenic T cells have a paradoxical protective effect in murine autoimmune diabetes by boosting Tregs. J. Clin. Invest. 120, 4558-4568. https://doi.org/10.1172/JCI42945.

Guo, X., Zhang, Y., Zheng, L., Zheng, C., Song, J., Zhang, Q., Kang, B., Liu, Z., Jin, L., Xing, R., Gao, R., Zhang, L., Dong, M., Hu, X., Ren, X., Kirchhoff, D., Roider, H. G., Yan, T., Zhang, Z., 2018a. Global characterization of T cells in non-small-cell lung cancer by single-cell sequencing. Nat. Med. 24, 978. https://doi.org/10.1038/s41591-018-0045-3.

Gupta, R., Babb, J. S., Singh, B., Chiriboga, L., Liebes, L., Adams, S., Demaria, S., 2011. The Numbers of FoxP3+ Lymphocytes in Sentinel Lymph Nodes of Breast Cancer Patients Correlate With Primary Tumor Size but Not Nodal Status. Cancer Invest. 29, 419-425. https://doi.org/10.3109/07357907.2011.585193.

Hori, S., Nomura, T., Sakaguchi, S., 2003. Control of Regulatory T Cell Development by the Transcription Factor Foxp3. Science 299, 1057-1061. https://doi.org/10.1126/science.1079490.

Kenefeck, R., Wang, C. J., Kapadi, T., Wardzinski, L., Attridge, K., Clough, L. E., Heuts, F., Kogimtzis, A., Patel, S., Rosenthal, M., Ono, M., Sansom, D. M., Narendran, P., Walker, L. S. K., 2015. Follicular helper T cell signature in type 1 diabetes. J. Clin. Invest. 125, 292-303. https://doi.org/10.1172/JCI76238.

Kohrt, H. E., Nouri, N., Nowels, K., Johnson, D., Holmes, S., Lee, P. P., 2005. Profile of Immune Cells in Axillary Lymph Nodes Predicts Disease-Free Survival in Breast Cancer. PLOS Med. 2, e284. https://doi.org/10.1371/iournal.pmed.0020284.

Lee, H.-M., Bautista, J. L., Hsieh, C.-S., 2011. Chapter 2—Thymic and Peripheral Differentiation of Regulatory T Cells, in: Alexander, R., Shimon, S. (Eds.), Advances in Immunology, Regulatory T-Cells. Academic Press, pp. 25-71. https://doi.org/10.1016/B978-0-12-387827-4.00002-4.

Lee, W., Lee, G. R., 2018. Transcriptional regulation and development of regulatory T cells. Exp. Mol. Med. 50, e456. https://doi.org/10.1038/emm.2017.313.

Lin, X., Chen, M., Liu, Y., Guo, Z., He, X., Brand, D., Zheng, S. G., 2013. Advances in distinguishing natural from induced Foxp3+ regulatory T cells. Int. J. Clin. Exp. Pathol. 6, 116-123.

Liyanage, U. K., Moore, T. T., Joo, H.-G., Tanaka, Y., Herrmann, V., Doherty, G., Drebin, J. A., Strasberg, S. M., Eberlein, T. J., Goedegebuure, P. S., Linehan, D. C., 2002. Prevalence of Regulatory T Cells Is Increased in Peripheral Blood and Tumor Microenvironment of Patients with Pancreas or Breast Adenocarcinoma. J. Immunol. 169, 2756-2761. https://doi.org/10.4049/jimmuno1.169.5.2756.

Lönnberg, T., Svensson, V., James, K. R., Fernandez-Ruiz, D., Sebina, I., Montandon, R., Soon, M. S. F., Fogg, L. G., Nair, A. S., Liligeto, U. N., Stubbington, M. J. T., Ly, L.-H., Bagger, F. O., Zwiessele, M., Lawrence, N. D., Souza-Fonseca-Guimaraes, F., Bunn, P. T., Engwerda, C. R., Heath, W. R., Billker, O., Stegle, O., Hague, A., Teichmann, S. A., 2017. Single-cell RNA-seq and computational analysis using temporal mixture modeling resolves TH1/TFH fate bifurcation in malaria. Sci. Immunol. 2, eaa12192. https://doi.org/10.1126/sciimmunol.aa12192.

Lutsiak, M. E. C., 2005. Inhibition of CD4+25+T regulatory cell function implicated in enhanced immune response by low-dose cyclophosphamide. Blood 105, 2862-2868. https://doi.org/10.1182/blood-2004-06-2410.

Mahmoud, S. M. A., Paish, E. C., Powe, D. G., Macmillan, R. D., Grainge, M. J., Lee, A. H. S., Ellis, I. O., Green, A. R., 2011. Tumor-Infiltrating CD8+ Lymphocytes Predict Clinical Outcome in Breast Cancer. J. Clin. Oncol. 29, 1949-1955. https://doi.org/10.1200/JC0.2010.30.5037.

Manno, G. L., Soldatov, R., Zeisel, A., Braun, E., Hochgerner, H., Petukhov, V., Lidschreiber, K., Kastriti, M. E., Lonnerberg, P., Furlan, A., Fan, J., Borm, L. E., Liu, Z., Bruggen, D. van, Guo, J., He, X., Barker, R., Sundstrom, E., Castelo-Branco, G., Cramer, P., Adameyko, I., Linnarsson, S., Kharchenko, P. V., 2018. RNA velocity of single cells. Nature 560, 494. https://doi.org/10.1038/s41586-018-0414-6.

Marschall, A. L., Dube′, S., Boldicke, T., 2015. Specific in vivo knockdown of protein function by intrabodies. MAbs. 2015; 7(6):1010-35. https://doi.org/10.1080/19420862.2015.1076601.

Mason, G. M., Lowe, K., Melchiotti, R., Ellis, R., de Rinaldis, E., Peakman, M., Heck, S., Lombardi, G., Tree, T. I. M., 2015. Phenotypic Complexity of the Human Regulatory T Cell Compartment Revealed by Mass Cytometry. J. Immunol. 195, 2030-2037. https://doi.org/10.4049/jimmuno1.1500703.

Maury, S., Lemoine, F. M., Hicheri, Y., Rosenzwajg, M., Badoual, C., Cherai, M., Beaumont, J.-L., Azar, N., Dhedin, N., Sirvent, A., Buzyn, A., Rubio, M.-T., Vigouroux, S., Montagne, O., Bories, D., Roudot-Thoraval, F., Vernant, J.-P., Cordonnier, C., Klatzmann, D., Cohen, J. L., 2010. CD4+CD25+ Regulatory T Cell Depletion Improves the Graft-Versus-Tumor Effect of Donor Lymphocytes After Allogeneic Hematopoietic Stem Cell Transplantation. Sci. Transl. Med. 2, 41ra52-41ra52. https://doi.org/10.1126/scitranslmed.3001302.

McInnes, L., Healy, J., 2018. UMAP: Uniform Manifold Approximation and Projection for Dimension Reduction, ArXiv e-prints 1802.03426, 2018. https://arxiv.org/abs/1802.03426.

Merlo, A., Casalini, P., Carcangiu, M. L., Malventano, C., Triulzi, T., Ménard, S., Tagliabue, E., Balsari, A., 2009. FOXP3 Expression and Overall Survival in Breast Cancer. J. Clin. Oncol. 27, 1746-1752. https://doi.org/10.1200/JCO.2008.17.9036.

Mouawad, R., Spano, J.-P., Khayat, D., 2011. Lymphocyte Infiltration in Breast Cancer: A Key Prognostic Factor That Should Not Be Ignored. J. Clin. Oncol. 29, 1935-1936. https://doi.org/10.1200/JCO.2011.35.4845.

Nakamura, R., Sakakibara, M., Nagashima, T., Sangai, T., Arai, M., Fujimori, T., Takano, S., Shida, T., Nakatani, Y., Miyazaki, M., 2009. Accumulation of regulatory T cells in sentinel lymph nodes is a prognostic predictor in patients with node-negative breast cancer. Eur. J. Cancer 45, 2123-2131. https://doi.org/10.1016/j.ejca.2009.03.024.

Ohara, M., Yamaguchi, Y., Matsuura, K., Murakami, S., Arihiro, K., Okada, M., 2009. Possible involvement of regulatory T cells in tumor onset and progression in primary breast cancer. Cancer Immunol. Immunother. 58, 441-447. https://doi.org/10.1007/s00262-008-0570-x.

Pérol, L., Martin, G. H., Maury, S., Cohen, J. L., Piaggio, E., 2014. Potential limitations of IL-2 administration for the treatment of experimental acute graft-versus-host disease. Immunol. Lett., Dutch Society for Immunology 1964-2014 162, 173-184. https://doi.org/10.1016/j.imlet.2014.10.027.

Pérol, L., Piaggio, E., 2016. New Molecular and Cellular Mechanisms of Tolerance: Tolerogenic Actions of IL-2, in: Cuturi, M. C., Anegon, I. (Eds.), Suppression and Regulation of Immune Responses. Springer New York, N. Y., N. Y., pp. 11-28. https://doi.org/10.1007/978-1-4939-3139-2_2.

Plitas, G., Konopacki, C., Wu, K., Bos, P. D., Morrow, M., Putintseva, E. V., Chudakov, D. M., Rudensky, A. Y., 2016. Regulatory T Cells Exhibit Distinct Features in Human Breast Cancer. Immunity 45, 1122-1134. https://doi.org/10.1016/j.immuni.2016.10.032.

Qiu, X., Hill, A., Packer, J., Lin, D., Ma, Y.-A., Trapnell, C., 2017. Single-cell mRNA quantification and differential analysis with Census. Nat. Methods 14, 309-315. https://doi.org/10.1038/nmeth.4150.

Sakaguchi, S., Wing, K., Onishi, Y., Prieto-Martin, P., Yamaguchi, T., 2009. Regulatory T cells: how do they suppress immune responses? Int. Immunol. 21, 1105-1111. https://doi.org/10.1093/intimm/dxp095.

Seki, A., Rutz, S., 2018. Optimized RNP transfection for highly efficient CRISPR/Cas9-mediated gene knockout in primary T cells. J. Exp. Med. 215, 985-997. https://doi.org/10.1084/jem.20171626.

Shifrut, E., Carnevale, J., Tobin, V., Roth, T. L., Woo, J. M., Bui, C. T., Li, P. J., Diolaiti, M. E., Ashworth, A., Marson, A., 2018. Genome-wide CRISPR Screens in Primary Human T Cells Reveal Key Regulators of Immune Function. Cell 175, 1958-1971.e15. https://doi.org/10.1016/j.cell.2018.10.024.

Stuart, T., Butler, A., Hoffman, P., Hafemeister, C., Papalexi, E., Mauck, W. M.3rd, Hao, Y., Stoeckius, M., Smibert, P., Satija, R., 2019. Comprehensive Integration of Single-Cell Data. Cell. 2019 Jun. 13; 177(7):1888-1902.e21. https://doi.org/10.1016/j.cell.2019.05.031.

Stubbington, M. J., Mahata, B., Svensson, V., Deonarine, A., Nissen, J. K., Betz, A. G., Teichmann, S. A., 2015. An atlas of mouse CD4(+) T cell transcriptomes. Biol Direct. 2015 Apr. 3; 10:14. https://doi.org/10.1186/s13062-015-0045-x.

Sun, S., Fei, X., Mao, Y., Wang, X., Garfield, D. H., Huang, O., Wang, Jinglong, Yuan, F., Sun, L., Yu, Q., Jin, X., Wang, Jianhua, Shen, K., 2014. PD-1+ immune cell infiltration inversely correlates with survival of operable breast cancer patients. Cancer Immunol. Immunother. 63, 395-406. https://doi.org/10.1007/s00262-014-1519-x.

Svensson V, Vento-Tormo R, Teichmann S A, 2018. Exponential scaling of single-cell RNA-seq in the past decade. Nat Protoc. 2018 April; 13(4):599-604. doi: 10.1038/nprot.2017.149. Epub 2018 Mar. 1.

Tang, F., Barbacioru, C., Wang, Y., Nordman, E., Lee, C., Xu, N., Wang, X., Bodeau, J., Tuch, B. B., Siddiqui, A., Lao, K., Surani, M. A., 2009. mRNA-Seq whole-transcriptome analysis of a single cell. Nat Methods. 2009 May; 6(5):377-82. https://doi.org/10.1038/nmeth.1315.

Tran, D. Q., Ramsey, H., Shevach, E. M., 2007. Induction of FOXP3 expression in naive human CD4+ FOXP3— T cells by T-cell receptor stimulation is transforming growth factor-β-dependent but does not confer a regulatory phenotype. Blood 110, 2983-2990. https://doi.org/10.1182/blood-2007-06-094656.

Tuerk, C., Gold, L., 1990. Systematic evolution of ligands by exponential enrichment: RNA ligands to bacteriophage T4 DNA polymerase. Science. 1990 Aug. 3; 249(4968):505-10. https://doi.org/10.1126/science.2200121.

Vento-Tormo, R., Efremova, M., Botting, R. A., Turco, M. Y., Vento-Tormo, M., Meyer, K. B., Park, J.-E., Stephenson, E., Polanski, K., Goncalves, A., Gardner, L., Holmqvist, S., Henriksson, J., Zou, A., Sharkey, A. M., Millar, B., Innes, B., Wood, L., Wilbrey-Clark, A., Payne, R. P., Ivarsson, M. A., Lisgo, S., Filby, A., Rowitch, D. H., Bulmer, J. N., Wright, G. J., Stubbington, M. J. T., Haniffa, M., Moffett, A., Teichmann, S. A., 2018. Single-cell reconstruction of the early maternal—fetal interface in humans. Nature 563, 347. https://doi.org/10.1038/s41586-018-0698-6.

Villarreal, D. O., L'Huillier, A., Armington, S., Mottershead, C., Filippova, E. V., Coder, B. D., Petit, R. G., Princiotta, M. F., 2018. Targeting CCR8 Induces Protective Antitumor Immunity and Enhances Vaccine-Induced Responses in Colon Cancer. Cancer Res. 78, 5340-5348. https://doi.org/10.1158/0008-5472.CAN-18-1119.

Wang, Z., Gerstein, M., Snyder, M., 2009. RNA-Seq: a revolutionary tool for transcriptions. Nat Rev GeLet. 2009 Jo E; 10(1):57-63. https://doi.org/10.1038/nrg2484.

Wang, D., Quiros, J., Mahuron, K., Pai, C.-C., Ranzani, V., Young, A., Silveria, S., Harwin, T., Abnousian, A., Pagani, M., Rosenblum, M. D., Van Gool, F., Fong, L., Bluestone, J. A., DuPage, M., 2018. Targeting EZH2 Reprograms Intratumoral Regulatory T Cells to Enhance Cancer Immunity. Cell Rep. 23, 3262-3274. https://doi.org/10.1016/j.celrep.2018.05.050.

Wolf, A. M., Wolf, D., Steurer, M., Gastl, G., Gunsilius, E., Grubeck-Loebenstein, B., 2003. Increase of Regulatory T Cells in the Peripheral Blood of Cancer Patients. Clin. Cancer Res. 9, 606-612.

Xu, K., Yang, W. Y., Nanayakkara, G. K., Shao, Y., Yang, F., Hu, W., Choi, E. T., Wang, H., Yang, X., 2018. GATA3, HDAC6, and BCL6 Regulate FOXP3+ Treg Plasticity and Determine Treg Conversion into Either Novel Antigen-Presenting Cell-Like Treg or Thl-Treg. Front. Immunol. 9. https://doi.org/10.3389/fimmu.2018.00045.

Zappia, L., Oshlack, A., 2018. Clustering trees: a visualization for evaluating clusterings at multiple resolutio CI. Gigascie Cie. 2018 Jul. 1; 7(7). https://doi.org/10.1093/gigascience/giy083.

Zemmour, D., Zilionis, R., Kiner, E., Klein, A. M., Mathis, D., Benoist, C., 2018. Single-cell gene expression reveals a landscape of regulatory T cell phenotypes shaped by the TCR. Nat. Immunol. 19, 291-301. https://doi.org/10.1038/s41590-018-0051-0.

Zhang, W., Ferguson, J., Ng, S. M., Hui, K., Goh, G., Lin, A., Esplugues, E., Flavell, R. A., Abraham, C., Zhao, H., Cho J. H., 2012. Effector CD4+ T cell expression signatures and immune-mediated disease associated genes. PLoS One. 2012; 7(6):e38510. https://doi.org/10.1371/journal.pone.0038510.

Zhang, L., Yu, X., Zheng, L., Zhang, Y., Li, Y., Fang, Q., Gao, R., Kang, B., Zhang, Q., Huang, J. Y., Konno, H., Guo, X., Ye, Y., Gao, S., Wang, S., Hu, X., Ren, X., Shen, Z., Ouyang, W., Zhang, Z., 2018. Lineage tracking reveals dynamic relationships of T cells in colorectal cancer. Nature 564, 268. https://doi.org/10.1038/s41586-018-0694-x.

Zhang, Q., Lu, W., Liang, C.-L., Chen, Y., Liu, H., Qiu, F., Dai, Z., 2018. Chimeric Antigen Receptor (CAR) Treg: A Promising Approach to Inducing Immunological Tolerance. Front. Immunol. 9, 2359. https://doi.org/10.3389/fimmu.2018.02359.

Zheng, G. X. Y., Terry, J. M., Belgrader, P., Ryvkin, P., Bent, Z. W., Wilson, R., Ziraldo, S. B., Wheeler, T. D., McDermott, G. P., Zhu, J., Gregory, M. T., Shuga, J., Montesclaros, L., Underwood, J. G., Masquelier, D. A., Nishimura, S. Y., Schnall-Levin, M., Wyatt, P. W., Hindson, C. M., Bharadwaj, R., Wong, A., Ness, K. D., Beppu, L. W., Deeg, H. J., McFarland, C., Loeb, K. R., Valente, W. J., Ericson, N. G., Stevens, E. A., Radich, J. P., Mikkelsen, T. S., Hindson, B. J., Bielas, J. H., 2017. Massively parallel digital transcriptional profiling of single cells. Nat. Commun. 8, 14049. https://doi.org/10.1038/ncomms14049.

Zhou, X., Bailey-Bucktrout, S. L., Jeker, L. T., Penaranda, C., Martinez-Llordella, M., Ashby, M., Nakayama, M., Rosenthal, W., Bluestone, J. A., 2009. Instability of the transcription factor Foxp3 leads to the generation of pathogenic memory T cells in vivo. Nat. Immunol. 10, 1000-1007. https://doi.org/10.1038/ni.1774.

Zhou X, Bailey-Bucktrout S L, Jeker L T, Penaranda C, Martinez-Llordella M, Ashby M, Nakayama M, Rosenthal W, Bluestone J A, 2009. Instability of the transcription factor Foxp3 leads to the generation of pathogenic memory T cells in vivo. Nat Immunol. 2009 September; 10(9):1000-7. doi: 10.1038/ni.1774. Epub 2009 Jul. 26.

Zuckerman, N. S., Yu, H., Simons, D. L., Bhattacharya, N., Carcamo-Cavazos, V., Yan, N., Dirbas, F. M., Johnson, D. L., Schwartz, E. J., Lee, P. P., 2013. Altered local and systemic immune profiles underlie lymph node metastasis in breast cancer patients. Int. J. Cancer 132, 2537-2547. https://doi.org/10.1002/ijc.27933.

1-23. (canceled)

24. A method of identification of functional disease-specific regulatory T cell markers, comprising the steps of:
a) Preparing a mixture of isolated regulatory T (Treg) cells and conventional T (Tconv) cells in similar proportions from at least a patient diseased-tissue sample and a patient peripheral blood sample;
b) Performing single-cell gene expression profiling combined with T cell receptor (TCR) profiling on each mixture of isolated Treg and Tconv cells from at least diseased-tissue and peripheral blood;
c) Identifying clusters of Treg cells and Tconv cells, wherein the clusters comprise differentially expressed genes or gene signatures between each other;
d) Determining at least one cluster of functional disease-specific Treg cells among the identified clusters of Treg cells, wherein the at least one cluster comprises:
(i) a higher proportion of Treg cells in the diseased-tissue than in the peripheral blood;
(ii) a higher proportion of Treg cells with clonally expanded TCR specificities in the diseased-tissue; and
(iii) a higher proportion of Treg cells with a transcriptomic signature of TCR triggering, cell activation and expansion in the diseased-tissue; and
e) Identifying genes that are differentially expressed in the cluster of functional disease-specific Treg cells in comparison with all the other identified clusters of Treg and Tconv cells.

25. The method according to claim 24, wherein the patient diseased-tissue sample is patient tumor sample and/or the patient samples in step (a) comprise a patient tumor sample, a patient tumor draining lymph node sample and a patient peripheral blood sample.

26. The method according to claim 24, wherein the mixture is composed of about 50% of Tconv cells and about 50% of Treg cells.

27. The method according to claim 24, wherein the combined single-cell gene expression profiling and T cell receptor (TCR) profiling in step (b) is performed by single-cell RNA sequencing method.

28. The method according to claim 24, wherein the at least one cluster of functional disease-specific Treg cells comprises a higher proportion of Treg cells overexpressing one or more of: REL, NKKB2, NR4A1, OX-40, 4-1BB, MHC class II molecules, in particular HLA-DR; CD39, CD137 and GITR.

29. The method according to claim 24, wherein said disease is a cancer selected from the group comprising: non-small cell lung cancer (NSCLC); breast, skin, ovarian, kidney and head and neck cancers; and rhabdoid tumors.

30. The method according to claim 24, wherein said disease is chosen from acute or chronic inflammatory, allergic, autoimmune or infectious diseases, graft-versus-host disease, graft-rejection.

31. The method according to claim 24, further comprising the identification and ranking of tumor-specific Treg markers for therapeutic purpose, according to the following steps:
Step 1: Identifying and selecting a fraction of n differentially expressed genes which code for a cell-membrane protein;
Step 2: Determining the average expression level of the n selected genes in normal tissue and assigning at least one score A to each gene from −1 for the gene having the lowest expression level to −n for the gene having the highest expression level in normal tissue;
Step 3: Determining the average expression level of the n selected genes in tumoral tissue and assigning at least one score B to each gene from +n for the gene having the highest expression level to +1 for the gene having the lowest expression level in tumoral tissue;
Step 4: Determining the average expression level of the n selected genes in normal PBMCs except Tregs and assigning at least one score C to each gene from +n for the gene having the lowest expression level to +1 for the gene having the highest expression level in normal PBMCs except Tregs;
Step 5: Determining the average expression level of the n selected genes in the tumor environment except Tregs and assigning at least one score D to each gene from +n for the gene having the lowest expression level to +1 for the gene having the highest expression level in tumor environment except Tregs;
Step 6: Determining the relative expression level of the n selected genes in i) Tumor-Tregs compared to Normal tissue-Tregs, and ii) Tregs compared to Tconvs and assigning two scores E and F to each gene from +n for the gene having the highest fold change expression level to +1 for the gene having the lowest fold change in i) (score E) Tumor Treg compared to normal adjacent tissue Treg, and ii) (score F) Tregs compared to Tconv; Step 7: Summating the assigned scores to obtain a cumulative assessment value (SUM SCORE) for each gene; and
Step 8: Determining the candidate therapeutic targets based on the cumulative assessment value.

32. The method according to claim 31, wherein the cell-membrane protein is a transmembrane or GPI-anchored protein with an extracellular domain.

33. A gene signature of functional tumor-specific Treg cells identified by the method according to claim 24, comprising the combination of up-regulated and down-regulated genes listed in Table 1.

34. A molecular marker for the detection, inactivation or depletion of tumor-specific Treg cells identified by the method according to claim 24, which is selected from the genes of Table 1 and their RNA or protein products.

35. A molecular marker for the detection, inactivation or depletion of tumor-specific Treg cells identified by the method according to claim 24, which is a cell-surface marker selected from the group consisting of: ADORA2A, CALR, CCR8, CD4, CD7, CD74, CD80, CD82, CD83, CSF1, CTLA4, CXCR3, HLA-B, HLA-DQA1, HLA-DR such as HLA-DRB5, ICAM1, ICOS, IGFLR1, IL12RB2, IL1R2, IL21R, IL2RA, IL2RB, IL2RG, LRRC32, NDFIP2, NINJ1, NTRK1, SDC4, SLC1A5, SLC3A2, SLC7A5, SLCO4A1, TMPRSS6, TNFRSF18, TNFRSF1B, TNFRSF4, TNFRSF8, TNFRSF9, TSPAN13 and TSPAN17.

36. A molecular marker for the detection, inactivation or depletion of tumor-specific Treg cells identified by the method according to claim 24, which is Vitamin D receptor (VDR).

37. A molecular marker for the detection, inactivation or depletion of tumor-specific Treg cells identified by the method according to claim 24, which is a therapeutic target modulating the viability, proliferation, stability or suppressive function of functional tumor-specific Treg cells is selected from the genes of Table 1 and their RNA or protein products.

38. A method of treating cancer, comprising administering to a patient in need thereof a therapeutically effective amount of:
(i) a modulator targeting at least one gene of Table 1 or RNA or protein product thereof, wherein the modulator is selected from the group comprising: small organic molecules, aptamers, antibodies, anti-sense oligonucleotides, interfering RNAs, ribozymes, and other agonists or antagonists such as dominant negative mutants or functional fragments of a therapeutic target protein, or
(ii) a cytotoxic agent comprising an antibody which binds to a tumor-specific Treg cell surface marker from Table 1 or a functional fragment thereof comprising the antigen binding site, coupled to a cytotoxic compound.

39. The method according to claim 38, wherein the cytotoxic agent inactivates or depletes tumor-specific Treg cells in vivo or ex vivo.

40. An engineered Treg cell which is defective for at least one of the up-regulated genes of Table 1 or over-expresses at least one of the down-regulated genes of Table 1.

41. The engineered Treg cell according to claim 40, which further comprises at least one genetically engineered antigen receptor that specifically binds a target antigen.
